(19) United States
(12) Patent Application Publication
(10) Pub. No.: US 2024/0165164 A1
(43) Pub. Date: May 23, 2024

(54) STEM CELLS FOR USE IN REDUCING THE IMMUNE RESPONSE FOLLOWING ORGAN TRANSPLANTATION

(71) Applicant: Amniotics AB, Lund (SE)

(72) Inventors: Jan Talts, Lund (SE); Sandra Lindstedt Ingemansson, Lund (SE)

(21) Appl. No.: 18/551,075

(22) PCT Filed: Mar. 18, 2022
(86) PCT No.: PCT/EP2022/057244

§ 371 Date: Sep. 18, 2023

(30) Foreign Application Priority Data
Mar. 19, 2021 (GB) 2103890.6

(51) Int. Cl. A61K 35/28; A61P 37/06

(52) U.S. Cl. CPC A61K 35/28; A61P 37/06

(57) ABSTRACT

The present invention relates to the mesenchymal stem cells and their use in modulating the immune response following transplantation.

### FIELD OF INVENTION

The present invention relates to the field of stem cells and their use in modulating the immune response following transplantation.

### BACKGROUND

Transplantation is a lifesaving therapy that may be the only curative treatment for patients suffering from a variety of end-stage lung diseases. Although the number of lung transplants being performed each year is increasing, transplantation waiting lists continue to increase and exceed the number of available donors, resulting in a huge unmet need in lung transplantation. For example, in 2017 it was reported that only 15-20% of lungs from multiorgan donors are considered usable for transplantation (Mariscal et al., 2017). Mariscal et al. highlights that despite various improvements in lung preservation, surgical technique, immunosuppression, and post-transplantation management, median survival after lung transplantation is only 6 years, with primary graft dysfunction (PGD) being the most serious early complication.

Ex vivo lung perfusion (EVLP) techniques have been developed to prolong ex vivo lung life, thereby increasing the window of opportunity for transporting donor lungs to a recipient. These techniques are also used to recondition lungs from a state that would have been deemed unsuitable for transplantation, thereby increasing the availability of transplantable organs. While advancements to EVLP may increase the eligibility of a lung being considered for transplant, advancements are also required during and following transplantation to reduce the risk of lung dysfunction, such as PGD, developing.

Mesenchymal stem cells (MSCs) can be found in nearly all tissues and are mostly located in perivascular niches. As will be understood by one of skill in the art, MSCs are multipotent stromal cells capable of differentiating into numerous cell types, and also possessing anti-inflammatory, angiogenic properties for directing tissue repair processes, thereby making MSCs valuable for therapeutic treatments. Term amniotic fluid (TAF) collected during a caesarean section contains a number of valuable cells, including MSCs. Moreover, specific subpopulations of MSCs are likely to be particularly well suited to use for production of therapeutic drugs. Previously, MSCs sourced from adult bone marrow, adult adipose tissue or neonatal birth-associated tissues including placenta, umbilical cord and cord blood were extensively used to obtain MSCs. MSCs from these neonatal tissues may have additional capacities in comparison to MSCs derived from adult sources. Indeed, several studies have reported superior biological properties such as improved proliferative capacity, life span and differentiation potential of MSCs from birth-associated tissues over adult derived MSCs. However, neither of these neonatal MSC sources have a corresponding tissue or organ in the adult body. Therefore, a neonatal quality MSCs with tissue specificity would be extremely beneficial. Moreover, acquisition of fetal material may be linked to negative consequences for the infant. For example, in cord blood harvesting it has been shown that as much of the cord blood as possible should be returned to the infant for improved survival, growth and fine motor skills development.

MSCs have been considered in EVLP and lung transplantation. For example, Nakajima et al., 2019 adapted the Toronto EVLP technique in a porcine model by introducing 5×10⁶ human umbilical cord perivascular MSCs during EVLP, and observed reduced lung tissue wet-to-dry weight ratio, indicating reduced oedema, and lower lung tissue TNFα, a cytokine involved in inflammation, 4 hours following transplantation. However, Nakajima et al. acknowledge that a limitation of their study is that the observed time of 4 hours after transplantation is short, and that further study with a longer observation time is needed to determine the ongoing immunomodulatory effects of MSCs on ischemia-reperfusion injury, which leads to PGD. Thus, there is a need in the art to develop new methods and/or to identify alternative sources of MSC's that may be applied to decrease the likelihood of e.g. PGD after transplantation.

The listing or discussion of an apparently prior-published document in this specification should not necessarily be taken as an acknowledgement that the document is part of the state of the art or is common general knowledge.

### SUMMARY OF INVENTION

Accordingly, it is an object of the present invention to advance the assessment of MSCs in lung transplantation and the potential for the development of PGD and/or graft versus host disease (GVHD). It is a further objection of the present invention to identify alternative and improved types of MSCs for use in improving lung transplantation. The type of MSCs characterised herein are derived from term amniotic fluid (TAF MSCs), which have been further characterised by panels of markers to identify a lung-specific subset of TAF MSCs. Term amniotic fluid (TAF) collected during a caesarean section contains a number of valuable cells, including MSCs. Amniotic fluid is today considered medical waste that is discarded. Therefore, both the ethical and practical incentive to harvest such an untapped resource is clear.

Certain disclosed examples relate to devices, cells, methods, uses, and systems for amniotic mesenchymal stem cells from amniotic fluid and cells derived thereof in EVLP. It will be understood by one of skill in the art that application of the devices, methods, uses, and systems described herein are not limited to a particular cell or tissue type. Further examples are described below.

A first aspect of the invention relates to an isolated term amniotic fluid (TAF) mesenchymal stem cells (MSCs) for use in modulating an immune response in a subject after tissue and/or organ transplantation.

A second aspect of the invention relates to an anti-rejection composition comprising isolated TAF MSCs for use in modulating an immune response in a subject after tissue and/or organ transplantation.

A third aspect of the invention relates to isolated TAF MSCs or an anti-rejection composition comprising isolated TAF MSCs for use in treating, reducing and/or preventing transplant rejection of a donor tissue and/or a donor organ.

A fourth aspect of the invention relates to isolated term amniotic fluid (TAF) mesenchymal stem cells (MSCs) for use in preventing and/or treating graft dysfunction and/or graft versus host disease (GVHD) in a subject after tissue and/or organ transplantation. In some embodiments, the isolated TAF MSCs are for use in preventing and/or treating graft dysfunction in a subject after tissue and/or organ transplantation. In some embodiments, the isolated TAF MSCs are for use in preventing and/or treating GVDH in a subject after tissue and/or organ transplantation.

A fifth aspect of the invention relates to a method of treating graft dysfunction and/or GVHD using the isolated TAF MSCs as described herein. treating graft dysfunction in a subject after tissue and/or organ transplantation.

A sixth aspect of the invention relates to use of isolated TAF MSCs as described herein in the preparation of a medicament for the treatment of graft dysfunction and/or GVHD.

A seventh aspect of the invention relates to an anti-rejection composition comprising isolated TAF MSCs for use in preventing and/or treating graft dysfunction and/or graft versus host disease (GVHD) in a subject after tissue and/or organ transplantation. In some embodiments, the composition comprising isolated TAF MSCs is for use in preventing and/or treating graft dysfunction in a subject after tissue and/or organ transplantation. In some embodiments, the composition comprising isolated TAF MSCs is for use in preventing and/or treating GVDH in a subject after tissue and/or organ transplantation.

An eighth aspect of the invention relates to a method of treating PGD and/or GVHD using the composition comprising isolated TAF MSCs as described herein.

A ninth aspect of the invention relates to use of a composition comprising isolated TAF MSCs as described herein in the preparation of a medicament for the treatment of PGD and/or GVHD.

### DESCRIPTION OF THE FIGURES

FIG. 1: A flow diagram showing the steps in the purification, culturing and selection of MSC subpopulations.

FIG. 2: A diagram illustrating a method for collecting amniotic fluid.

FIG. 3: A schematic illustration, in a perspective view, of an apparatus for filtering amniotic fluid according to an example.

FIG. 4: A schematic illustration, in a cross-sectional side view, of an apparatus for filtering amniotic fluid according to an example.

FIG. 5: A schematic illustration, in a cross-sectional side view, of an apparatus for filtering amniotic fluid according to an example.

FIG. 6: A schematic illustration, in a cross-sectional side view, of an apparatus for filtering amniotic fluid according to an example.

FIG. 7: A schematic illustration, in a cross-sectional side view, of an apparatus for filtering amniotic fluid according to an example.

FIG. 8: A schematic illustration, in a cross-sectional side view, of an apparatus for filtering amniotic fluid according to an example.

FIG. 9: A schematic illustration, in a cross-sectional side view, of an apparatus for filtering amniotic fluid according to an example.

FIG. 10: A schematic illustration, in a cross-sectional side view, of an apparatus for filtering amniotic fluid according to an example.

FIG. 11: A schematic illustration, in a cross-sectional side view, of an apparatus for filtering amniotic fluid according to an example.

FIG. 12: (a) A schematic illustration, in a cross-sectional side view, of an apparatus for filtering amniotic fluid according to an example. (b) A schematic illustration, along a cross-section A-A in FIG. 10, of an apparatus for filtering amniotic fluid according to an example.

FIG. 13: A flow chart of a method of filtering amniotic fluid according to an example.

FIG. 14: A flow chart showing the steps for calculation of an MSC tissue specificity score according to an example.

FIG. 15: An example graph showing MSC tissue specificity scores representing the 5% and 15% thresholds.

FIG. 16: An example graph showing tissue-prioritized and tissue-distal data, including tissue-prioritized data greater than 15% percentile.

FIG. 17: (A)-(D) show the results of an example study demonstrating the effects of using TAF Lung MSCs to treat rats with induced lung fibrosis.

FIG. 18: Overview of the experimental setup. The experimental setup for the porcine model for EVLP and lung transplantation is portrayed. The grey circles represent a time point at which a particular sample was obtained: (A) hemodynamic and arterial blood gas (ABG); (B) plasma sample obtained using a Sysmex KX-21N automated haematology analyser; (C) biopsy; and (D) bronchoalveolar lavage fluid (BALF). The syringe indicates a time point at which MSCs were administered.

FIG. 19: Establishment of an ARDS model in pigs. Pigs treated with LPS were assessed for various biomarkers to confirm establishment of ARDS. (a) Analysis of the concentration of TNF-α, IL-10, IL-1B, IL-8, IL-6, IL-12, IFN-α, IFN-γ and IL-4 in pigs treated with LPS at 60 minutes, 120 minutes and upon confirmation of ARDS. (b) Concentrations of the cytokines listed in (a) detected in BALF at baseline (prior to LPS) and upon confirmation of ARDS. (c) Cell count for neutrophils, lymphocytes and total white blood cells before LPS, 30 minutes after LPS, 60 minutes after LPS and upon confirmation of ARDS. (d) Histological analysis of a lung section at baseline (prior to LPS) and in ARDS lung injury. (e) Clinical score of the histology shown in (d).

FIG. 20: IL-1B concentration. The concentration of IL-1ß in plasma at various time points during EVLP. The treated group have been treated with MSCs; and the non-treated group received a PBS control.

FIG. 21: IFN-α concentration. The concentration of IFN-α in plasma at various time points during EVLP. The treated group have been treated with MSCs; and the non-treated group received a PBS control.

FIG. 22: IL-8 concentration. The concentration of IL-8 in plasma at various time points following lung transplantation (LTx). The treated group have been treated with MSCs; and the non-treated group received a PBS control. “LTx end 4 h” indicates 4 hours following the right pneumonectomy (referred to as “4 Hr Monitoring” in FIG. 1).

FIG. 23: TNF-α concentration. The concentration of TNF-α in plasma at various time points following lung transplantation (LTx). The treated group have been treated with MSCs; and the non-treated group received a PBS control. “LTx end 4 h” indicates 4 hours following the right pneumonectomy (referred to as “4 Hr Monitoring” in FIG. 1).

FIG. 24: IL-1B concentration. The concentration of IL-1ß in plasma at various time points following lung transplantation (LTx). The treated group have been treated with MSCs; and the non-treated group received a PBS control. “LTx end 4 h” indicates 4 hours following the right pneumonectomy (referred to as “4 Hr Monitoring” in FIG. 1).

FIG. 25: IL-4 concentration. The concentration of IL-4 in plasma at various time points following lung transplantation (LTx). The treated group have been treated with MSCs; and the non-treated group received a PBS control. “LTx end 4 h” indicates 4 hours following the right pneumonectomy (referred to as “4 Hr Monitoring” in FIG. 1).

FIG. 26: Lymphocyte count after 24 hours. The number of lymphocytes per millilitre of blood were counted and compared between pigs treated with MSCs (“A Treatment”) and control pigs that received PBS as a placebo treatment. Statistical significance is indicated by * and was calculated as described in the “Calculations and statistics” section.

FIG. 27: Assessment of primary graft dysfunction (PGD) in EVLP and lung transplantation (LTx). The ratio (“kvot”) of arterial oxygen partial pressure (PaO₂ in mmHg) to fractional inspired oxygen (FiO₂) is assessed for EVLP and LTx. The baseline indicates the PaO₂/FiO₂ ratio in pigs prior to LPS treatment. Acute Respiratory Distress Syndrome (ARDS) indicates the PaO₂/FiO₂ ratio in pigs following LPS treatment. For EVLP and LTx, “Treatment” indicates that the pigs received MSCs and “No Treatment” indicates that the pigs received a PBS control. Statistical significance is indicated by * and was calculated as described in the “Calculations and statistics” section. A ratio between 201-300 mmHg is defined as “moderate PGD 2”, and a ratio of 0-200 mmHg is defined as “severe PGD 3”.

FIG. 28: (Table 1) Plate layout—Two plates of each layout were prepared. One plate for cell composition and cytokine (FACS/Luminex) analysis and one plate for cell composition and proliferation (CFSE) analysis. PBMCs and MSCs were added at indicated ratios to columns 1, 2, 3, 6, 7, 8, 11, 12 but not to columns 4, 5, 9, 10.

FIG. 29: Gating strategy used for all samples, analysed after 24 hours of activation, to identify specific PBMC subpopulations and to exclude MSCs. The figure shows gating strategy for PBMC:MSC sample (1:2.5) activated with aCD3/aCD2B in cell composition plates. A) Gating of single cells, B) gating of lymphocytes and granulocytes, C) NOT gating on non T cells, D) gating of CD4+ and CD8+ cells among T cells, E) gating of PD-1+ and CD73+ cells among CD4+ lymphocytes (double positives are not included), F) gating of PD-1+ and CD73+ cells among CD8+ lymphocytes (double positives are not included), G) gating of PD-1+ and CD73+ cells among CD4+ lymphocytes, H) gating of PD-1+ and CD73+ cells among COB+ lymphocytes, I) gating of CD80+ cells among granulocytes and macrophages, J) gating of CD73+ cells among CD80+ granulocytes and macrophages, K) gating of CD206+ cells among granulocytes and macrophages, L) gating of CD163+ cells among CD206+ granulocytes and macrophages and M) gating of CD73+ cells among CD163+CD206+ granulocytes and macrophages. Back-gating was performed to verify lymphocyte and granulocyte/macrophage gates.

FIG. 30: Representative figures showing gating of CD4+ and CD8+ cells among T cells for all PBMC:MSC ratios. (FSC vs SSC and CD4 vs CD8) for PBMC:MSC ratios 1:0, 1:2.5, 1:5, 1:10 and 0:1, activated for 24 hours with aCD3/aCD28 in the cell composition plate. From the FSC vs SSC dot plot-lymphocyte gate, a NOT gate was set on non-T cells and CD4+ and CD8+ T cells were analysed.

FIG. 31: T cell activation status after co-culturing PBMCs with MSCs or reference drugs. PBMCs and MSCs were co-cultured in different PBMC:MSC ratios and activated with aCD3/aCD28. Cell populations were analysed using flow cytometry after 24 hours of incubation. Results show A) % CD4+ among lymphocytes, B) Expression of CD4 on T cells (MFI), C) % PD-1+ among CD4+ lymphocytes, D) Expression of PD-1 on CD4+ lymphocytes (MFI), E) % PD-1+ among CD4+ lymphocytes (including double positives), F) Expression of PD-1 on CD4+ lymphocytes (MFI) (including double positives), G) % CD73+ among CD4+ lymphocytes, H) Expression of CD73 on CD4+ lymphocytes (MFI), % CD73+ among CD4+ lymphocytes (including double positives), J) Expression of CD73 on CD4+ lymphocytes (MFI) (including double positives), K) % CD8 among lymphocytes, L) Expression of CD8 on T cells (MFI), M) % PD-1+ among CD8+ lymphocytes, N) Expression of PD-1 on CD8+ lymphocytes (MFI), 0) % PD-7+ among CD8+ lymphocytes (including double positives), P) Expression of PD-1 on CD8+ lymphocytes (MFI) (including double positives), Q) % CD73+ among CD8+ lymphocytes, R) Expression of CD73 on CD8+ lymphocytes (MF1), S) % CD73+ among CD8+ lymphocytes (including double positives) and T) Expression of CD73 on CD8+ lymphocytes (MFI) (including double positives). Results are presented as mean values (of % or median fluorescent intensity)+/−SEM.

FIG. 32: Macrophage activation status after coculturing PBMCs with MSCs or reference drugs. PBMCs and MSCs were co-cultured in different PBMC:MSC ratios and activated with aCD3/aCD28. Cell populations were analysed using flow cytometry after 24 hours of incubation. Results show A) % CD80+ among granulocytes and macrophages, B) Expression of CD80 on granulocytes and macrophages (MR), C) % CD73+ among CD80+ granulocytes and macrophages, D) Expression of CD73 on CD80+ granulocytes and macrophages (MEI), E) % CD163+CD206+ among granulocytes and macrophages, F) Expression of CD163 and CD206 on granulocytes and macrophages (MEI), G) % CD73+ among CD163+CD206+ granulocytes and macrophages, H) Expression of CD73 on CD163+CD206+ granulocytes and macrophages, Results are presented as mean values (of % or median fluorescent intensity)+/−SEM.

FIG. 33: Gating strategy used for all samples, analysed after 72 hours of activation, to identify specific PBMC subpopulations and to measure proliferation using CFSE labeling. CFSE labeling of PBMCs was performed before coculture with MSCs, therefore all CFSE positive cells are PBMCs. The figure shows gating strategy for PBMC:MSC sample (1:2.5) activated with aCD3/aCD28 in CFSE proliferation plates. A) Gating of single cells and B) gating of lymphocytes and granulocytes/macrophages. Gating for CD4+ cells and CD8+ cells was performed as for the 24-hour activation samples. Back-gating was performed to verify lymphocyte and granulocyte/macrophage gates.

FIG. 34: Histograms showing the gating strategy for CFSE plates. A) Gating of CFSE proliferation among CD4+ lymphocytes, B) gating of CFSE proliferation among CD8+ lymphocytes, C) gating of CFSE proliferation among CD80+ granulocytes and macrophages and D) gating of CFSE proliferation among CD163+CD206+ granulocytes and macrophages. Horizontal bars delineate proliferating cells. E) shows CFSE zero peak.

FIG. 35: Representative graphs of CFSE proliferation in CD4+ PBMCs co-cultured with MSCs in media 3 and analysed using flow cytometry after 72 hours of incubation. PBMCs and MSCs were co-cultured in different ratios (PBMC:MSC—0:1, 1:10, 1:5, 1:2.5, and 1:0).

FIG. 36: A) Representative graphs of CFSE proliferation in CD4+ PBMCs co-cultured with MSCs in media 3 and analysed using flow cytometry after 72 hours of incubation. PBMCs and MSCs were co-cultured in different ratios (PBMC:MSC—0:1, 1:10, 1:5, 1:2.5, and 1.0). B) Representative graphs of CFSE proliferation in CD8+ PBMCs co-cultured with MSCs in media 3 and analysed using flow cytometry after 72 hours of incubation. PBMCs and MSCs were co-cultured in different ratios (PBMC:MSC—0:1, 1:10, 1:5, 1:2.5, and 1:0). C) and D) Representative graphs of CFSE proliferation in CD80+ cells (M1) and in CD163+CD206+ cells (M2), analysed using flow cytometry after 72 hours of incubation. Representative graphs show PBMCs and MSCs co-cultured in different ratios (PBMC:MSC—0:1, 1:10, 1:5, 1:2.5 and 1:0) cultured in media 3.

FIG. 37: A) and B) Representative graphs of CFSE proliferation in CD80+ cells (M1) and in CD163+CD206+ cells (M2), analysed using flow cytometry after 72 hours of incubation. Representative graphs show PBMCs and MSCs co-cultured in different ratios (PBMC:MSC—0:1, 1:10, 1:5, 1:2, 5 and 1:0) cultured in media 3.

FIG. 38: T cell composition and proliferation status after coculturing PBMCs with MSCs or reference drugs. PBMCs and MSCs were co-cultured in different PBMC:MSC ratios and activated with aCD3/aCD28. Cell populations were analysed using flow cytometry after 72 hours of incubation. Results show A) % CD4+ among T cells, B) Expression of CD4 on T cells (MFI), C) Proliferating CD4+ among T cells, D) MSCs inhibitory effect on CD4+ cells, E) % CD8+ among T cells, F) Expression of CD8 on T cells (MFI), G) Proliferating CD8+ among T cells and H) MSCs inhibitory effect on CD8+ cells. Results are presented as mean values+/−SEM. CFSE zero peak has been removed from analysis. Proliferation has been analysed using the geometric mean (Geo mean) value which describes the MFI (mean fluorescent intensity) in a logarithmic histogram. The number of events in each fluorescent channel is divided by the number of channels but since the scale is logarithmic, arithmetic values cannot be used. The Geo mean compensates for the logarithmic scale and is also considering bright and dim populations.

FIG. 39: Macrophage composition and proliferation status after coculturing PBMCs with MSCs or reference drugs. PBMCs and MSCs were co-cultured in different PBMC:MSC ratios and activated with aCD3/aCD28. Cell populations were analysed using flow cytometry after 72 hours of incubation. Results show A) % CD80+ among granulocytes and macrophages, B) Expression of CD80 on granulocytes and macrophages (MFI), C) Proliferating CD80+ among granulocytes and macrophages, D) MSCs inhibitory effect on CD80+ cells, E) % CD163+CD206+ among granulocytes and macrophages, F) Expression of CD163 and CD206 on granulocytes and macrophages (MFI), G) Proliferating CD163+CD206+ among granulocytes and macrophages and H) MSCs inhibitory effect on CD163+CD206+ cells. Results are presented as mean values (of % or median fluorescent intensity)+/−SEM. Proliferation has been analysed using the geometric mean (Geo mean) value which describes the MFI (mean fluorescent intensity) in a logarithmic histogram. The number of events in each fluorescent channel is divided by the number of channels but since the scale is logarithmic, arithmetic values cannot be used. The Geo mean compensates for the logarithmic scale and is also considering bright and dim populations.

FIG. 40: Cytokine analysis after co-culturing PBMCs with MSCs or reference drugs. PBMCs and MSCs were co-cultured in different PBMC:MSC ratios and activated with aCD3/aCD28. Cytokine levels in supernatants from cells in FACS/Luminex plates were analysed using Luminex after 24 hours of incubation. Results show levels of A) IGF, B) CXCL9, C) IL-10, D) IFN-α, E) HGF, F) IL-6 (FI), G) IL-18, H) IFN-g, I) VEGF (FI), J) TNF-α, K) IL-12/IL-23p40, L) b-NGF, in all tested PBMC:MSC ratios in media 3. Results are presented as mean values+/−SEM. For some of the analysed cytokines, IFN-α values are below the dynamic range and IL-6 and VEGF values are above the dynamic range and results are therefore presented as FI levels (fluorescence intensity).

FIG. 41: Cytokine levels were analysed in supernatants from aCD3/aCD28 activated PBMCs alone, MSCs alone and in CM from all donors using Luminex after 24 hours of incubation. Results show A) CXCL9 levels, B) TNF-α levels, C) IFN-α levels, D) IGFBP-1 levels, E) IL-10 levels, F) IL-18 levels, G) HGF levels, H) IFN-g levels, I) IL-6 levels, J) IL-12/23 levels, K) b-NGF levels and L) VEGF levels in supernatant using media 3. Results are presented as mean values+/−SEM. For some of the analysed cytokines, IFN-α values are below the dynamic range and IL-6 and VEGF values are above the dynamic range and results are therefore presented as FI levels (fluorescence intensity).

### DETAILED DESCRIPTION OF THE INVENTION

In some embodiments, the immune response is inflammation. The isolated TAF MSCs or anti-rejection compositions described herein may be anti-inflammatory by reducing T helper cell activation (or reducing the number of activated T helper cells); increasing regulatory Treg numbers and/or activity; reducing total number of T cells or effector T cells; and/or reducing macrophage activation or the number of activation macrophages. Alternatively, or additionally, the isolated TAF MSCs or anti-rejection compositions described herein may be anti-inflammatory by modulating innate immune cells (such as neutrophils, macrophages, monocytes, fibrocytes, mast cells, innate lymphoid cells (ILCs; e.g. type 2 ILCs), and/or dendritic cells); and/or adaptive immune cells (such as Th1 cells, Th2 cells, Th9 cells, Th17 cells, Tregs, and/or B cells); and/or any of the inflammatory processes associated with each.

The compositions referred to herein may be pharmaceutical compositions comprising at least one pharmaceutically acceptable carrier, excipient or further component such as therapeutic and/or prophylactic ingredient. A “pharmaceutically acceptable carrier” as referred to herein, is any known compound or combination of known compounds that are known to those skilled in the art to be useful in formulating pharmaceutical compositions. The carrier may include one or more excipients or diluents. Pharmaceutical compositions of the invention can be placed into dosage forms, such as in the form of unit dosages. Pharmaceutical compositions include those suitable for any route of administration (as discussed further below).

In some embodiments, the inflammation is selected from the group consisting of tissue-specific inflammation and organ-specific inflammation. In some embodiments, the inflammation may be in a tissue and/or organ selected from the group consisting of lung, kidney, neural, skin, liver, heart (and heart valves), trachea, body parts (such as limbs/digits), pancreas, intestine, colon and combinations thereof. Preferably, the inflammation to be treated is lung/pulmonary inflammation. In some embodiments, the inflammation may be systemic inflammation. In some embodiments, multiple types of inflammation may be occurring simultaneously. For example, the inflammation may be lung inflammation and heart inflammation, or lung inflammation and systemic inflammation.

In some embodiments, the donor tissue and/or donor organ according to any of the aspects described herein was obtained from a donor treated with isolated TAF MSCs. For example, the donor may have received isolated TAF MSCs or a composition comprising isolated TAF MSCs before (e.g. immediately before) the tissue or organ was retrieved from the donor. In some embodiments, the isolated TAF MSCs or composition comprising isolated TAF MSCs may be the same that the donor received as those used for the recipient of the donor tissue or organ. Alternatively, or additionally, the donor tissue and/or the donor organ may have been pre-treated ex-vivo with isolated TAF MSCs or a composition comprising isolated TAF MSCs. For example, the donor tissue and/or the donor organ may be removed from the donor and subsequently exposed to isolated TAF MSCs or a composition comprising isolated TAF MSCs prior to the donor tissue and/or the donor organ being transplanted into a recipient.

In some embodiments, the donor tissue and/or the donor organ has been transported ex-vivo in a conditioning media. Preferably, the conditioning media is a physiological conditioning media. The conditioning media may comprise isolated TAF MSCs and/or a composition comprising isolated TAF MSCs. In some embodiments, the conditioning media may further comprise one or more of the following components: dextran (e.g. dextran 40), red blood cells, and albumin (for example, human albumin). It may be appreciated that the conditioning media haematocrit (also referred to as the erythrocyte volume fraction) is at a concentration from 10 v/v % to 25 v/v %, for example from 15 v/v % to 25 v/v %, or 10 v/v %, 11 v/v %, 12 v/v %, 13 v/v %, 14 v/v %, 15 v/v %, 16 v/v %, 17 v/v %, 18 v/v %, 19 v/v %, 20 v/v %, 21 v/v %, 22 v/v %, 23 v/v %, 24 v/v %, or 25 v/v %. In a preferred embodiment the conditioning media haematocrit is 14 v/v %). Values considered normal for red blood cells in the blood are about 45 v/v % for males and about 40 v/v % for females. In some embodiments, albumin (e.g. human albumin, also referred to as human serum albumin (HSA)) is at a concentration from 1-25 v/v %, for example 5-25 v/v %, 1 v/v %, 5 v/v %, 10 v/v %, 15 v/v %, 20 v/v %, or 25 v/v %. In some embodiments, the conditioning media may further comprise one or more of the following components: at least one glucocorticoid (e.g. prednisolone and/or methylprednisolone), at least one anticoagulant (e.g. heparin), and at least one antibiotic. Conditioning media known in the art may be adapted to include TAF MSCs as a supplement. For example, solutions used in EVLP, such as Steen™ solution.

In some embodiments, the isolated TAF MSCs comprise tissue-specific markers and/or organ-specific markers, preferably wherein the tissue-specific markers and/or the organ-specific markers correspond to said donor tissue or donor organ. In some embodiments, the isolated TAF MSCs may be a mixed population of multiple subtypes of isolated TAF MSCs, in which case a portion of the isolated TAF MSCs comprise tissue-specific markers and/or organ-specific markers while another portion comprise different tissue-specific markers and/or organ-specific markers, preferably wherein at least one of the portions of tissue-specific markers and/or the organ-specific markers correspond to said donor tissue or donor organ.

In some embodiments, the donor tissue and/or donor organ is from a non-living subject. Preferably, the non-living subject is the same species as the intended recipient of the donor tissue and/or donor organ. For example, the tissue and/or organ may be obtained from a non-living human (also referred to as a corpse or cadaver) and is for transplantation in a human in need thereof. In some embodiments, the donor tissue and/or donor organ is from a living subject. Preferably, the living subject is the same species as the intended recipient of the donor tissue and/or donor organ. A donor tissue and/or donor organ provided by a living subject is limited to a donor tissue and/or donor organ that can be parted with from the donor without resulting in cessation of the donor's life. For example, a single kidney of a functional pair of kidneys could be donated, or a skin graft taken from an excess of skin.

In some embodiments, ex-vivo donor tissue and/or ex-vivo donor organ may be selected from the group consisting of a lung, kidney, neural, skin, liver, heart (and heart valves), trachea, pancreas, intestine, colon and body parts. Body parts may be any body part such as limbs (e.g. arms and legs) or digits. In a preferred embodiment, the ex-vivo donor tissue and/or ex-vivo donor organ is a lung. The ex-vivo donor tissue and/or ex-vivo donor organ may also be a portion of ex-vivo donor organs selected from the group consisting of a lung, kidney, neural, skin, liver, heart (and heart valves), trachea, pancreas, intestine, colon and body parts.

In the present context a tissue is a group of cells with a similar structure, organised to carry out specific functions. An organ is a collection of tissues that structurally form a functional unit specialised to perform a particular function. Accordingly, the term “portion thereof” with respect to an organ may refer to a tissue. Within the context of limbs and digits (in reattachment and/or re-enervation), the tissue and/or organ in question may be skin and/or a part of the nervous system. For example, reattachment of a digit may be a finger that has been separated from a subject, wherein the skin of the finger is reattached to the subject at the site from where it is lost. Alternatively, the digit may be from a donor, in which case it is attached in replacement of a limb or digit that a recipient has lost.

Even a small increase in the ex-vivo life of a donor organ and/or a donor tissue positively impacts the number of available transplantable organs as new geographical areas may be applied to supply donor organs and/or donor tissue. Thus, it may be preferred that the ex-vivo life of the ex-vivo donor tissue and/or ex-vivo donor organ is prolonged by at least 10 minutes, e.g. 20 minutes, such as 30 minutes, e.g. 40 minutes, such as 50 minutes, e.g. 1 hour, such as 2 hours compared to a control wherein the control is an ex-vivo donor tissue and/or ex-vivo donor organ not subjected to isolated TAF MSCs or a composition comprising isolated TAF MSCs.

In some embodiments, the ex-vivo donor tissue and/or ex-vivo donor organ remains viable outside the body for at least 1 hour, such as 2 hours, e.g. 4 hours, such as 6 hours, e.g. 8 hours, such as 10 hours, e.g. 12 hours, such as 14 hours, e.g. 16 hours, such as 18 hours, e.g. 20 hours, such as 22 hours, e.g. 1 days, such as 2 days. In the present context the term viability is to be understood as how long an ex-vivo donor tissue and/or ex-vivo donor organ can stay outside the body before the cell function begins to fail and the likelihood that the ex-vivo organ and/or ex-vivo tissue will malfunction in the recipient will increase. Transplant organ failure, known as primary graft dysfunction (PGD), is the “most feared complication” associated with organ transplants. Alternatively, or additionally, transplant organ failure may be associated with graft versus host disease (GVHD), in which the donor tissue and/or donor organ contains immune cells that react against the host recipient. Accordingly, the isolated TAF MSCs or compositions described herein may treat, prevent, or reduce the negative effects of PGD and/or GVHD.

The assessment of viability of a donor tissue and/or donor organ following transplantation depends on the tissue and/or organ. For example, the viability of the lung can be assessed based on the level of oxygenation achieved by the recipient following transplantation. Accordingly, an organ-specific assessment can be compared with the clinically accepted criteria for said organ-specific assessment. For example, oxygenation is an accepted standard for assessing lung function, so can be analysed in a recipient following lung transplantation and compared with relevant population data for the expected oxygenation for the subject, or in comparison to oxygenation achievable by the recipient prior to transplantation. Techniques for assessing organ function are known to the skilled person. In some embodiments, the assessment of viability may be characterised by improved organ graft function in the long term (e.g. 1, 2, 3, 4, 5, 6, 7, 8, 9, 10, 11 or 12 months post-transplantation) compared with organ function within one week following transplantation of the subject, or to a control subject having undergone a tissue and/or organ transplant and experiencing delayed graft function who has not been exposed to isolated TAF MSCs. Alternatively, or additionally, the control for comparison may be a tissue and/or organ that has not been exposed to isolated TAF MSCs. Viability may also be referred to as preservation.

While viability is an important parameter the state of the ex-vivo donor tissue and/or ex-vivo donor organ before transplantation is another important parameter. As can be seen in Example 2, IL-1B is a known biomarker for distinguishing between what would be considered an “organ suitable for transplantation” from an “unsuitable for transplantation”. In Example 2 the organ is a lung. IL-1B is an inducible proinflammatory cytokine that is not generally expressed in healthy cells or tissue. The release of IL-1B can cause pulmonary inflammation and fibrosis. Accordingly, a lower level of IL-1ß is a favourable outcome within the context of organ physiology and repair. Administration of isolated TAF MSCs or a composition comprising isolated TAF MSCs according to the present invention may be applied to change the organ status from “unsuitable for transplantation” to “suitable for transplantation”.

In some embodiments, the isolated TAF MSCs and/or compositions described herein have been introduced to an ex-vivo donor tissue and/or ex-vivo donor organ before and/or are introduced during transplantation to the recipient. For example, the isolated TAF MSCs and/or compositions described herein may have been introduced to an ex-vivo donor tissue and/or ex-vivo donor organ during perfusion of the donated tissue and/or organ. In lung transplantation, the isolated TAF MSCs and/or compositions described herein may have been introduced to an ex-vivo donor tissue and/or ex-vivo donor organ during EVLP.

Alternatively, or additionally, the isolated TAF MSCs and/or compositions described herein may be introduced to the ex-vivo donor tissue and/or ex-vivo donor organ at the time of transplantation and/or at an interval of time following completion of transplantation. For example, the isolated TAF MSCs and/or compositions described herein may be introduced to the ex-vivo donor tissue and/or ex-vivo donor organ 1 hour following transplantation. As a further example, the isolated TAF MSCs and/or compositions described herein may be introduced to the ex-vivo donor tissue and/or ex-vivo donor organ 12 hours following transplantation. As a further example, the isolated TAF MSCs and/or compositions described herein may be introduced to the ex-vivo donor tissue and/or ex-vivo donor organ 1 hour and 12 hours following transplantation. Each of these examples may be in addition to or replacement of the isolated TAF MSCs and/or compositions described herein being introduced at the time of transplantation. A subsequent administration of isolated TAF MSCs and/or compositions described herein may be to ‘top-up’ the levels of TAF MSCs or activity thereof. For example, a serum or biopsy sample from the donated tissue or organ may reveal that the concentration of an inflammatory cytokine has recovered from the TAF MSC-dependent reduction in its expression, which may be used to assess whether the recipient needs a top-up of TAF MSCs. Accordingly, subsequent administrations of isolated TAF MSCs and/or compositions described here may be in a subject in need thereof.

In some embodiments, the isolated TAF MSCs or composition comprising isolated TAF MSCs is administered in combination with a further agent, sequentially, simultaneously and/or subsequently. For example, the further agent may be administered as part of the composition comprising isolated TAF MSCs. In some embodiments, the further agent is selected from the group consisting of anti-inflammatory agents, immunosuppressive agents, anti-rejection agents/drugs (e.g. prednisone, tacrolimus, etc) and any combinations thereof. The term “anti-inflammatory agent” indicates that the agent or drug reduces or prevent an immune response that causes inflammation. The term “immunosuppressive agents” indicates that the agent or drug blocks or reduces the activity of an immune response, which may be a proinflammatory or anti-inflammatory response. By “anti-rejection composition” we include the term “anti-rejection drug”. This term is commonly used in the art to refer to immunosuppressants, particularly those used to treat, prevent and/or reduce transplant rejection. Therefore, the term “anti-rejection composition” includes the meaning of an immunosuppressant that prevents and/or reduces pathologies associated with transplant rejection. The isolated TAF MSCs and compositions described herein may be used to replace or supplement (i.e. used in combination) other anti-rejection drugs that have failed to treat, prevent, and/or reduce transplant rejection. An agent or drug may fall within the definition of any one or more of these terms, and so the terms may be used herein interchangeably.

In some embodiments, the isolated TAF MSCs or composition comprising isolated TAF MSCs are administered more than once. For example, administration may occur 2, 3, 4, 5, 6, 7, 8, 9, 10 or more times. In a preferred embodiment, administration occurs once during EVLP and at least once (for example, twice) following transplantation.

In some embodiments, the isolated TAF MSCs or composition comprising isolated TAF MSCs are administered at a concentration of 1-3 million cells per kg of the recipient, preferably in the range of 1.5-2.5 million cells per kg of the recipient, preferably 2 million cells per kg of the recipient. This may be achieved through a single administration of the intended dose or through multiple administrations amounting to a total corresponding to the intended dose.

As used herein, the term “administering” or “administration”, refers to the placement of isolated TAF MSCs or a composition as disclosed herein into a subject by a method or route which results in at least partial localisation of the agents or composition at a desired site. “Route of administration” may refer to any administration pathway known in the art, including but not limited to oral, topical, aerosol, nasal, via inhalation, anal, intra-anal, peri-anal, transmucosal, transdermal, parenteral, enteral, or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the agent or composition may be in the form of solutions or suspensions for infusion or for injection. Via the enteral route, the agent or composition can be in the form of capsules, gel capsules, syrups, suspensions, solutions, emulsions, or lipid vesicles or polymer vesicles allowing controlled release. Via the topical route, the agent or composition can be in the form of aerosol, lotion, cream, gel, ointment, suspensions, solutions or emulsions.

In some embodiments, the isolated TAF MSCs or composition comprising isolated TAF MSCs are administered intravenously, intraarterially, intravascularly and/or intrabronchially. In a preferred embodiment, the TAF MSCs are administered intravenously. The site of intravenous administration is preferably upstream of the transplantation site. For example, in lung transplantation, intravenous administration is preferably upstream of the lung. In some embodiments, isolated TAF MSCs are administered to the lung an EVLP system, for example via tubing associated with an EVLP system.

It will be appreciated that administration may be before, during and/or after transplantation is performed. For example, administration before transplantation may be intravenous (IV) to the donor before the tissue or organ has been removed from the donor, either directly into the tissue or organ of interest and/or into the blood stream of the donor, preferably wherein the administration is directly into the tissue or organ of interest. Alternatively, or additionally, administration may be directly into the donor tissue or donor organ after it has been removed from the donor, and/or by submerging the donor tissue or donor organ into a conditioning media during transportation. For example, in EVLP, administration may be via IV administration directly into the donor tissue or donor organ (e.g. donor lung or donor lung tissue). In a particularly preferred embodiment, the IV administration is directly into the donor tissue or donor organ after its removal from the donor.

In some embodiments, the organ is subjected to an effective amount of isolated TAF MSCs about 30-36 hours, about 25-30 hours, about 20-25 hours, about 15-20 hours, about 10-15 hours, about 5-10 hours, about 1-5 hour or combinations thereof, prior to implantation of the organ in the subject. In some embodiments, the organ is treated with an effective amount of isolated TAF MSCs about 1 hour, 2 hours, 3 hours, 4 hours, 5 hours, 6 hours, 7 hours, 8 hours, 9 hours, 10 hours, 10-15 hours, 15-20 hours, 20-24 hours or combinations thereof, prior to implantation of the organ in the subject.

Administration during transplantation may be IV administration into the donor tissue or donor organ upon its transplantation into the tissue or organ recipient. In this context, “during” includes at any point during which a surgeon considers the transplantation process to be ongoing. For example, administration may be prior to the donor tissue or donor organ being inserted into a recipient but after the donor tissue or donor organ has been removed from a perfusion system or storage container. As a further example, administration may be simultaneous to the donor tissue or donor organ being grafted to a recipient or immediately after engraftment. Alternatively, or additionally, administration during transplantation may be IV administration into the bloodstream of the recipient while they are undergoing a transplantation procedure.

Administration after transplantation may be IV administration directly into the donor tissue or donor organ that has been grafted into the recipient, following a transplantation procedure. Alternatively, or additionally, administration after transplantation may be IV administration into the bloodstream of the recipient at any time following termination of a transplantation procedure. For example, this may be a continuation of the administration to the bloodstream that occurs during the transplantation procedure, immediately after the transplantation procedure, or hours after the transplantation procedure. In some embodiments, administration after transplantation may be at least 1 hour after transplantation, for example at least 2, 3, 4, 5, 6, 7, 8, 9, 10, 11, 12, 13, 14, 15, 16, 17, 18, 19, 20, 21, 22, 23 and/or 24 hours after transplantation. In a preferred embodiment, the administration may be 1 hour and 12 hours after transplantation. In various embodiments, the administration is up to any one or more of one month, two months, six months, twelve months, 18 months, 24 months or 30 months after transplant.

In some embodiments, delayed graft function (DGF) is observed in the subject that has undergone organ (e.g. lung) transplant. Known clinical interventions may be needed in the case of DGF, which may vary depending on the organ, e.g. dialysis may be needed in the subject within seven days of transplant for a kidney. In various embodiments, a reduction in the need for the intervention is observed about 2 weeks, 3 weeks or 4 weeks after the transplant. In further embodiments, the reduction in the need for the intervention is observed about 2-4 weeks, 1-3 months, 3-6 months, 6-9 months, 9-12 months or 12-15 months after the transplant.

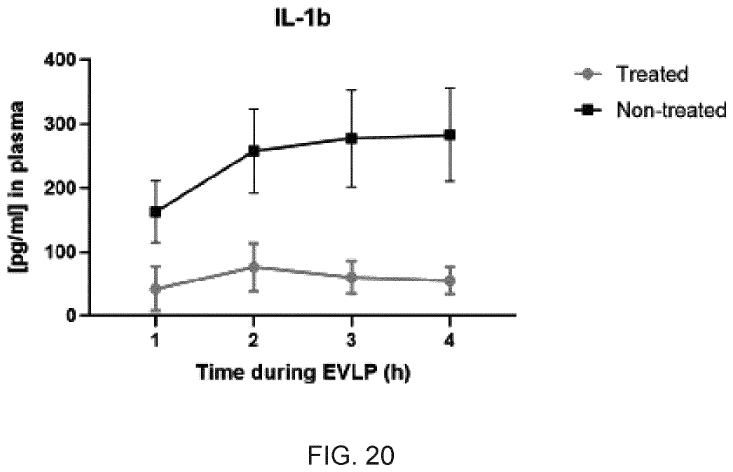

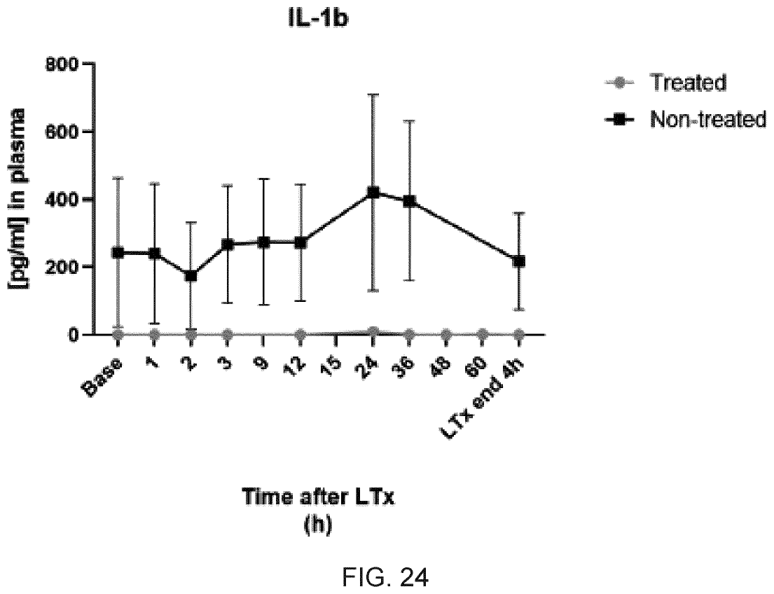

In some embodiments, the concentration of IL1-beta (IL-1B) present in the ex-vivo donor tissue and/or ex-vivo donor organ is reduced compared to a control following administration of isolated TAF MSCs or a composition comprising isolated TAF MSCs, wherein the control is not exposed to isolated TAF MSCs (see e.g. FIG. 20 for EVLP and FIG. 24 for following transplantation). The reduction in IL-1B may be assessed at least 1 hour following administration, for example 2, 3, 4, 5, 6, 7, 8, 9, 10, 11, 12, 13, 14, 15, 16, 17, 18, 19, 20, 21, 22, 23, 24, 36, 48, 60, 64 or more hours following administration. Alternatively, or additionally, the reduction in IL-1B may be assessed after a total of at least 1 hour of cold ischemia time (i.e. the time at which a donor tissue and/or donor organ is preserved at a cold temperature, such as at 4° C. or between 8-12° C.), for example a total of at least 2, 3, 4, 5, 6, 7, 8, 9, 10, 11, 12, 13, 14, 15, 16, 17, 18, 19, 20, 21, 22, 23, 24, 36, 48, 60, 64 or more hours of cold ischemia time.

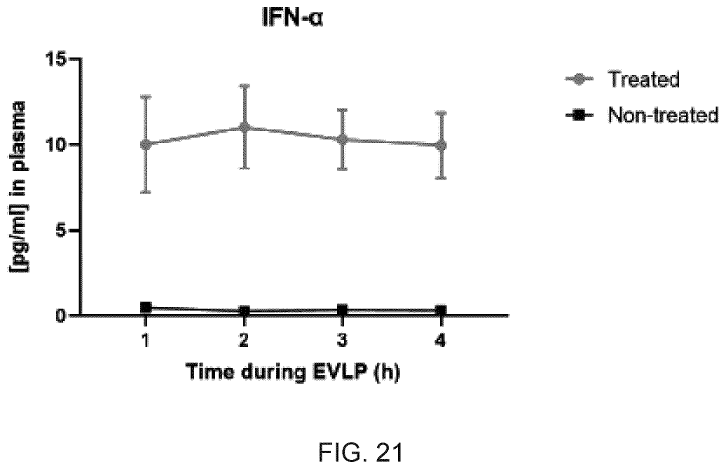

In some embodiments, the concentration of IFN-alpha (IFN-α) present in the ex-vivo donor tissue and/or ex-vivo donor organ is increased compared to a control following administration of isolated TAF MSCs or a composition comprising isolated TAF MSCs, wherein the control is not exposed to isolated TAF MSCs (see e.g. FIG. 21). The increase in IFN-α may be assessed at least 1 hour following administration, for example 2, 3, 4, 5, 6, 7, 8, 9, 10, 11, 12, 13, 14, 15, 16, 17, 18, 19, 20, 21, 22, 23, 24, 36, 48, 60, 64 or more hours following administration. Alternatively, or additionally, the increase in IFN-α may be assessed after a total of at least 1 hour of cold ischemia time (i.e. the time at which a donor tissue and/or donor organ is preserved at a cold temperature, such as at 4° C. or between 8-12° C.), for example a total of at least 2, 3, 4, 5, 6, 7, 8, 9, 10, 11, 12, 13, 14, 15, 16, 17, 18, 19, 20, 21, 22, 23, 24, 36, 48, 60, 64 or more hours of cold ischemia time.

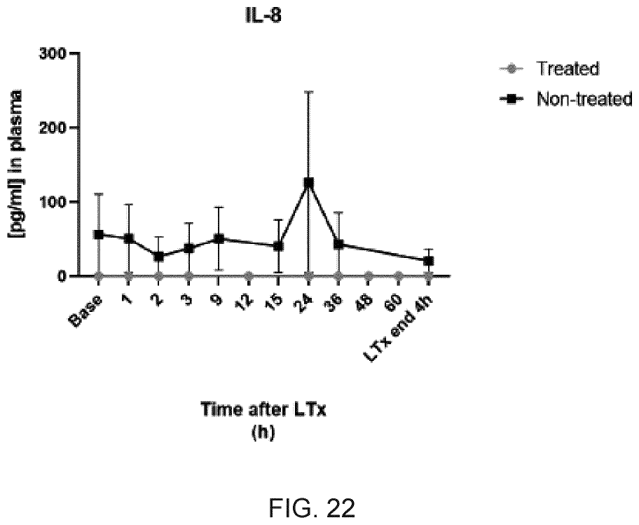

In some embodiments, the concentration of IL-8 present in the ex-vivo donor tissue and/or ex-vivo donor organ is reduced compared to a control following administration of isolated TAF MSCs or a composition comprising isolated TAF MSCs, wherein the control is not exposed to isolated TAF MSCs (see e.g. FIG. 22). The reduction in IL-8 may be assessed at least 1 hour following administration, for example 2, 3, 4, 5, 6, 7, 8, 9, 10, 11, 12, 13, 14, 15, 16, 17, 18, 19, 20, 21, 22, 23, 24, 36, 48, 60, 64 or more hours following administration. Alternatively, or additionally, the reduction in IL-8 may be assessed after a total of at least 1 hour of cold ischemia time (i.e. the time at which a donor tissue and/or donor organ is preserved at a cold temperature, such as at 4° C. or between 8-12° C.), for example a total of at least 2, 3, 4, 5, 6, 7, 8, 9, 10, 11, 12, 13, 14, 15, 16, 17, 18, 19, 20, 21, 22, 23, 24, 36, 48, 60, 64 or more hours of cold ischemia time.

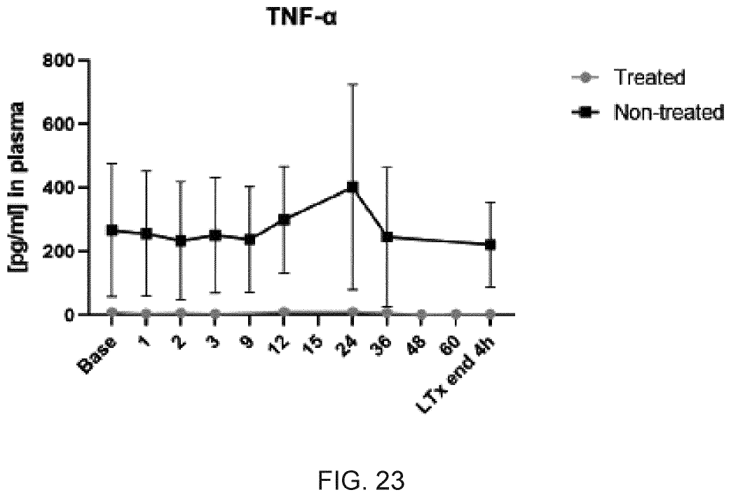

In some embodiments, the concentration of TNF-α present in the ex-vivo donor tissue and/or ex-vivo donor organ is reduced compared to a control following administration of isolated TAF MSCs or a composition comprising isolated TAF MSCs, wherein the control is not exposed to isolated TAF MSCs (see e.g. FIG. 23). The reduction in TNF-α may be assessed at least 1 hour following administration, for example 2, 3, 4, 5, 6, 7, 8, 9, 10, 11, 12, 13, 14, 15, 16, 17, 18, 19, 20, 21, 22, 23, 24, 36, 48, 60, 64 or more hours following administration. Alternatively, or additionally, the reduction in TNF-α may be assessed after a total of at least 1 hour of cold ischemia time (i.e. the time at which a donor tissue and/or donor organ is preserved at a cold temperature, such as at 4° C. or between 8-12° C.), for example a total of at least 2, 3, 4, 5, 6, 7, 8, 9, 10, 11, 12, 13, 14, 15, 16, 17, 18, 19, 20, 21, 22, 23, 24, 36, 48, 60, 64 or more hours of cold ischemia time.

In some embodiments, the concentration of IL-4 present in the ex-vivo donor tissue and/or ex-vivo donor organ is reduced compared to a control following administration of isolated TAF MSCs or a composition comprising isolated TAF MSCs, wherein the control is not exposed to isolated TAF MSCs (see e.g. FIG. 22). The reduction in IL-4 may be assessed at least 1 hour following administration, for example 2, 3, 4, 5, 6, 7, 8, 9, 10, 11, 12, 13, 14, 15, 16, 17, 18, 19, 20, 21, 22, 23, 24, 36, 48, 60, 64 or more hours following administration. Alternatively, or additionally, the reduction in IL-4 may be assessed after a total of at least 1 hour of cold ischemia time (i.e. the time at which a donor tissue and/or donor organ is preserved at a cold temperature, such as at 4° C. or between 8-12° C.), for example a total of at least 2, 3, 4, 5, 6, 7, 8, 9, 10, 11, 12, 13, 14, 15, 16, 17, 18, 19, 20, 21, 22, 23, 24, 36, 48, 60, 64 or more hours of cold ischemia time.

In situations where administration occurs multiple times, any of the readouts described herein may be with respect to any of the administrations. For example, the reduction in lymphocytes may be assessed 24 hours after an initial administration of isolated TAF MSCs, which may be 12 hours after a second or further administration of isolated TAF MSCs.

In some embodiments, the isolated TAF MSCs are derived from an MHC/HLA-matched donor. However, this is not essential. Although it is possible that the recipient's immune system may recognise MHC/HLA-mismatched MSCs, the immunosuppressive and immune-privileged properties of MSCs may permit their use in allogeneic transplantation (Mordant et al., 2016).

In some embodiments, the isolated TAF MSCs are a clonal population. By “clonal” we include the meaning that the population is generated from a single precursor cell. In some embodiments, the isolated TAF MSCs are a mix of clonal populations. The isolated TAF MSCs may be homogeneous (i.e. of uniform composition or character) or heterogeneous (i.e. not of uniform composition or character).

In some embodiments, the isolated TAF MSCs may be in a single-cell suspension. Alternatively, the isolated TAF MSCs may be pelleted and in need of re-suspension prior to subsequent use. Alternatively, or additionally, the isolated TAF MSCs may be in a frozen state, and therefore require thawing before use.

In some embodiments, the isolated TAF MSCs are capable of forming colony forming units (CFU) in culture. The skilled person is aware of culture conditions (e.g. 2D and 3D culture conditions) that can be used to assess whether MSCs are capable of forming CFU in culture.

In some embodiments, the isolated TAF MSCs are functionally characterised. For example, the isolated TAF MSCs may be characterised based on the release of cytokines implicated in growth stimulation or suppression; and/or differentiation into osteogenic, chondrogenic or adipogenic cell lineages), which indicates their ability to differentiate into bone, cartilage or fat lineages. MSCs may be characterised by plastic adhesion, expression of certain cell surface markers (e.g. receptors), absence of other cell surface markers (e.g. receptors), and/or their ability to differentiate into bone, cartilage and fat lineages. In some embodiments, the TAF MSCs have a likeness to certain tissue type MSCs, e.g. lung. By “likeness” we include the meaning that the TAF MSCs express similar or the same markers to the tissue type MSCs, express similar or the same cytokine profiles, lack expression of similar or the same markers, and/or functionally behave in a similar or the same way.

In some embodiments, the isolated TAF MSCs are functionally characterised based on fewer activated T helper cells, more regulatory T cells, fewer activated cytotoxic T cells, and/or fewer effector T cells following co-culture with PBMCs, in comparison with PBMC controls not treated with TAF MSCs (as shown in Example 3). In some embodiments, the isolated TAF MSCs are functionally characterised based on fewer activated macrophages (e.g. M1 and/or M2 macrophages) following co-culture with PBMCs, in comparison with PBMC controls not treated with TAF MSCs (as shown in Example 4).

In some embodiments, the isolated TAF MSCs have been pre-sorted or enriched to contain markers of interest using the techniques described herein.

In some embodiments, the isolated TAF MSCs have been passaged multiple times. For example, the isolated TAF MSCs may have been passaged 1, 2, 3, 4, 5, 6, or more times.

Mesenchymal stem cells may be obtained from amniotic fluid by a method comprising: providing term amniotic fluid (TAF); removing particulate material from the TAF to obtain purified TAF cells; performing adherence selection on the purified TAF cells to obtain TAF adherence cells; passaging the TAF adherence cells to obtain TAF mesenchymal stem cells (TAF MSCs); and selecting TAF MSCs that express a marker selected from the group consisting of TBC1 domain family member 3K (TBC1D3K), allograft inflammatory factor 1 like (AIF1L), cadherin related family member 1 (CDHR1), sodium/potassium transporting ATPase interacting 4 (NKAIN4), ATP binding cassette subfamily B member 1 (ABCB1), plasmalemma vesicle associated protein (PLVAP), mesothelin (MSLN), L1 cell adhesion molecule (L1CAM), hepatitis A virus cellular receptor 1 (HAVCR1), mal, T cell differentiation protein 2 (gene/pseudogene) (MAL2), SLAM family member 7 (SLAMF7), double C2 domain beta (DOC2B), endothelial cell adhesion molecule (ESAM), gamma-aminobutyric acid type A receptor beta1 subunit (GABRB1), cadherin 16 (CDH16), immunoglobulin superfamily member 3 (IGSF3), desmocollin 3 (DSC3), regulator of hemoglobinization and erythroid cell expansion (RHEX), potassium voltage-gated channel interacting protein 1 (KCNIP1), CD70 molecule (CD70), GDNF family receptor alpha 1 (GFRA1), crumbs cell polarity complex component 3 (CRB3), claudin 1 (CLDN1), novel transcript (AC118754.1), sodium voltage-gated channel alpha subunit 5 (SCN5A), fibroblast growth factor receptor 4 (FGFR4), potassium two pore domain channel subfamily K member 3 (KCNK3), dysferlin (DYSF), ephrin A1 (EFNA1), potassium inwardly rectifying channel subfamily J member 16 (KCNJ16), membrane associated ring-CH-type finger 1 (MARCHF1), synaptotagmin like 1 (SYTL1), calsyntenin 2 (CLSTN2), integrin subunit beta 4 (ITGB4), vesicle associated membrane protein 8 (VAMP8), G protein-coupled receptor class C group 5 member C (GPRC5C), CD24 molecule (CD24), cadherin EGF LAG seven-pass G-type receptor 2 (CELSR2), cadherin 8 (CDH8), glutamate receptor interacting protein 1 (GRIP1), dematin actin binding protein (DMTN), F11 receptor (F11R), cell adhesion molecule 1 (CADM1), cadherin 6 (CDH6), coagulation factor II thrombin receptor like 2 (F2RL2), LY6/PLAUR domain containing 1 (LYPD1), solute carrier family 6 member 6 (SLC6A6), desmoglein 2 (DSG2), adhesion G protein-coupled receptor G1 (ADGRG1), cholecystokinin A receptor (CCKAR), oxytocin receptor (OXTR), integrin subunit alpha 3 (ITGA3), adhesion molecule with Ig like domain 2 (AMIGO2), cadherin EGF LAG seven-pass G-type receptor 1 (CELSR1), EPH receptor B2 (EPHB2). In another aspect, the isolated TAF MSCs are obtainable by the method according to the present disclosure, said cells expressing a surface marker selected from the group comprising of TBC1 domain family member 3K (TBC1D3K), allograft inflammatory factor 1 like (AIF1L), cadherin related family member 1 (CDHR1), sodium/potassium transporting ATPase interacting 4 (NKAIN4), ATP binding cassette subfamily B member 1 (ABCB1), plasmalemma vesicle associated protein (PLVAP), mesothelin (MSLN), L1 cell adhesion molecule (L1CAM), hepatitis A virus cellular receptor 1 (HAVCR1), mal, T cell differentiation protein 2 (gene/pseudogene) (MAL2), SLAM family member 7 (SLAMF7), double C2 domain beta (DOC2B), endothelial cell adhesion molecule (ESAM), gamma-aminobutyric acid type A receptor beta1 subunit (GABRB1), cadherin 16 (CDH16), immunoglobulin superfamily member 3 (IGSF3), desmocollin 3 (DSC3), regulator of hemoglobinization and erythroid cell expansion (RHEX), potassium voltage-gated channel interacting protein 1 (KCNIP1), CD70 molecule (CD70), GDNF family receptor alpha 1 (GFRA1), crumbs cell polarity complex component 3 (CRB3), claudin 1 (CLDN1), novel transcript (AC118754.1), sodium voltage-gated channel alpha subunit 5 (SCN5A), fibroblast growth factor receptor 4 (FGFR4), potassium two pore domain channel subfamily K member 3 (KCNK3), dysferlin (DYSF), ephrin A1 (EFNA1), potassium inwardly rectifying channel subfamily J member 16 (KCNJ16), membrane associated ring-CH-type finger 1 (MARCHF1), synaptotagmin like 1 (SYTL1), calsyntenin 2 (CLSTN2), integrin subunit beta 4 (ITGB4), vesicle associated membrane protein 8 (VAMP8), G protein-coupled receptor class C group 5 member C (GPRC5C), CD24 molecule (CD24), cadherin EGF LAG seven-pass G-type receptor 2 (CELSR2), cadherin 8 (CDH8), glutamate receptor interacting protein 1 (GRIP1), dematin actin binding protein (DMTN), F11 receptor (F11R), cell adhesion molecule 1 (CADM1), cadherin 6 (CDH6), coagulation factor II thrombin receptor like 2 (F2RL2), LY6/PLAUR domain containing 1 (LYPD1), solute carrier family 6 member 6 (SLC6A6), desmoglein 2 (DSG2), adhesion G protein-coupled receptor G1 (ADGRG1), cholecystokinin A receptor (CCKAR), oxytocin receptor (OXTR), integrin subunit alpha 3 (ITGA3), adhesion molecule with Ig like domain 2 (AMIGO2), cadherin EGF LAG seven-pass G-type receptor 1 (CELSR1), EPH receptor B2 (EPHB2). Alternatively, or additionally, a method for obtaining TAF MSCs from term amniotic fluid may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF MSCs; and selecting the TAF MSCs from the population as cells that express at least one Group A surface marker selected from the group consisting of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2, thereby obtaining the TAF MSCs.

In some embodiments, selecting TAF MSCs may comprise selecting TAF MSCs that have a reduced expression of markers selected from the group consisting of IL13RA2, CLU, TMEM119, CEMIP, LSP1, GPNMB, FAP, CRLF1, MME, CLMP, BGN, DDR2. Removing particulate matter may comprise filtering and centrifuging the TAF. Performing adherence selection on the purified TAF cells may comprise adhering the purified TAF cells to a surface coated with a vitronectin-based substrate. The selecting step may be performed using fluorescence activated cell sorting (FACS). The selecting step may be performed with antibodies directed to any of the markers or surface markers. The selecting step may comprise selecting TAF MSCs that express at least two markers from the Group A surface markers. The selecting step may comprise selecting TAF MSCs that express at least three markers from the Group A surface markers. The selecting step may comprise selecting TAF MSCs that express at least four markers from the Group A surface markers. The selecting step may comprise a plurality of sorting steps, each sorting step comprising directing TAF MSCs into a first output group or a second output group in dependence on a set of markers expressed or not expressed by the respective TAF MSCs.

In some embodiments, the selecting step may comprise a first sorting step to direct TAF MSCs that express a Group A surface marker into a first output group, and a second sorting step to direct TAF MSCs from the first output group that express a second set of markers into a second output group.

In certain embodiments, a method for obtaining term amniotic fluid lung mesenchymal stem cells (lung TAF MSCs) from term amniotic fluid, may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the lung TAF MSCs; and selecting the TAF lung MSCs from the population as cells that express at least one Group B surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1, thereby obtaining lung TAF MSCs.

Selecting lung TAF MSCs may comprise excluding MSCs that express a marker selected from the group consisting of CD24, ITGB4, TNFSF10, GFRA1, CD74, FGFR4, HAVCR1, and OSCAR. The selecting step may comprise selecting TAF MSCs that express at least two surface markers from the Group B surface markers. The selecting step may comprise selecting TAF MSCs that express at least three surface markers from the Group B surface markers. The selecting step may comprise selecting TAF MSCs that express at least four surface markers from the Group B surface markers. The selecting step may comprise selecting TAF MSCs that express a surface marker selected from the group of CD248, DDR1, and LRRC38. The selecting step may comprise selecting TAF MSCs that express CD248. The selecting step may comprise selecting TAF MSCs that express CD248 in combination with a marker selected from the group of DDR1 and LRRC38. The selecting step may comprise selecting TAF MSCs that express CD248, DDR1, and LRRC38. In some examples, isolated TAF MSCs may be obtainable by the methods described above, said cells expressing at least one Group A surface marker.

In some embodiments, an isolated population of TAF MSCs, may express at least one Group A surface marker selected from the group comprising of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2.

In some embodiments, a composition may comprise the isolated TAF MSCs described above and a pharmaceutically acceptable carrier for the TAF MSCs. Isolated lung TAF MSCs obtainable by a method described above may express at least one Group B surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18 and CRLF1. In certain examples, isolated lung TAF MSCs may express at least one Group B surface marker.

In some embodiments, a method for obtaining term amniotic fluid kidney mesenchymal stem (kidney TAF MSCs) cells from term amniotic fluid, may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF kidney MSCs; and selecting the TAF kidney MSCs from the population as cells that express at least one Group C surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1, thereby obtaining kidney TAF MSCs.

In certain embodiments, isolated kidney TAF MSCs may express at least one Group C surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1. A composition may comprise the isolated kidney TAF MSCs as described above.

In some embodiments, a method for obtaining term amniotic fluid skin mesenchymal stem cells (skin TAF MSCs) from term amniotic fluid may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF skin MSCs; and selecting the skin TAF MSCs from the population as cells that express at least one Group D surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2; and obtaining skin TAF MSCs.

In certain embodiments, isolated skin TAF MSCs may express at least one Group D surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2. A composition may comprise the isolated skin TAF MSCs described above and a pharmaceutically acceptable carrier for the skin TAF MSCs.

In some embodiments, a method for obtaining neural TAF MSCs from term amniotic fluid may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF neural MSCs; and selecting the TAF neural MSCs from the population as cells that express at least one Group E surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4; thereby obtaining neural TAF MSCs.

In some embodiments, an isolated population of neural TAF MSCs may express at least one Group E surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1 and ITGB4. A composition may comprise the isolated population of neural TAF MSCs described above and a pharmaceutically acceptable carrier for the neural TAF MSCs.

In some embodiments, the isolated TAF MSCs are between 15-25 μm diameter. In a preferred embodiment, the isolated TAF MSCs are between 18-22 μm diameter. By “between”, we intend to include the diameters specified at either end of a range. For example, “between 15-25 μm” may include isolated TAF MSCs that have a diameter of 15, 16, 17, 18, 19, 20, 21, 22, 23, 24 and/or 25 μm. In some embodiments, only a portion of the population are present between the aforementioned diameters. For example, in some embodiments, at least 70%, 80%, 90%, 95% or more of the total population are between 15-25 μm or 18-22 μm diameter. Alternatively, or additionally, at least 70%, 80%, 90%, 95% or more of the total population have a diameter that is more than 15, 16, 17, 18, 19, 20, 21, 22, 23 or 24 μm in diameter. Alternatively, or additionally, at least 70%, 80%, 90%, 95% or more of the total population have a diameter that is less than 25, 24, 23, 22, 21, 20, 19, 18, 17, 16 μm diameter. For example, in some embodiments, at least 70%, 80%, 90%, 95% or more of the total population of TAF MSCs are ≤25 μm or ≤22 μm diameter. For any of the aforementioned values or ranges thereof, it may be that the population of isolated TAF MSCs has an average diameter of the value or within the range. In some embodiments, the size or average size is determined by using a cell counter, such as the Nucleocounter 202 (NucleoCounter® NC-202™, Automated cell counter, chemometec).

In some embodiments, the isolated TAF MSCs comprise lower actin expression and/or fewer vesicles at the surface compared with adult MSCs. Suitable techniques for determining actin and/or vesicle levels are known to the skilled person, such as that described in Mo et al., 2017.

In some embodiments, the isolated TAF MSCs correspond to the lung TAF MSCs, kidney TAF MSCs, skin TAF MSCs, neural TAF MSCs, or combinations thereof, as described above. In some embodiments, the isolated TAF MSCs correspond to a population obtainable by the methods described herein.

In some embodiments, the isolated TAF MSCs or composition comprising isolated TAF MSCs is formed of a combination of different types of TAF MSCs. For example, a population may be formed by mixing isolated lung TAF MSCs with isolated kidney TAF MSCs. The combination of interest may depend on the requirements of the subject who will receive the cells. For example, a subject in need of multiple organ transplants (e.g. lung and kidney) may benefit from receiving a mixed population in which the most suitable types of TAF MSCs for the organs (e.g. lung TAF MSCs and kidney TAF MSCs) in question are mixed. In some embodiments, the mixed population may comprise at least 10%, 20%, 30%, 40%, 50%, 60%, 70%, 80%, 90%, 95%, or more lung TAF MSCs; at least 10%, 20%, 30%, 40%, 50%, 60%, 70%, 80%, 90%, 95%, or more kidney TAF MSCs; at least 10%, 20%, 30%, 40%, 50%, 60%, 70%, 80%, 90%, 95%, or more skin TAF MSCs; at least 10%, 20%, 30%, 40%, 50%, 60%, 70%, 80%, 90%, 95% or more neural TAF MSCs; and/or at least 10%, 20%, 30%, 40%, 50%, 60%, 70%, 80%, 90%, 95% or more of unsorted TAF MSCs. Percentage is calculated based on the total number of TAF MSC's. The percentage may be an integer between any of the specified values. For example, a mixed population may comprise at least 24% lung TAF MSCs, wherein the remaining percentage is a different type of TAF MSCs (such as unsorted TAF MSCs). As a further example, a mixed population may comprise at least 80% of a specific type of TAF MSCs (e.g. at least 80% of lung TAF MSCs), wherein the remaining percentage is a different type of TAF MSCs (e.g. unsorted TAF MSCs). In some embodiments, the minimum threshold for a particular type of TAF MSCs is 24%; for example, a mixed population may comprise a minimum of 24% of lung TAF MSCs. The percentage of a particular type of TAF MSCs may relate to any one or more of the markers described herein. For example, at least 24% lung TAF MSCs includes the meaning that, following MSC sorting, at least 24% of the cell population express CD248.

In a preferred embodiment the composition comprising isolated TAF MSCs comprises at least 10%, 20%, 30%, 40%, 50%, 60%, 70%, 80%, 90%, 95%, 96%, 97%, 98%, 99% or 100% lung TAF MSCs

Graft dysfunction as described herein may be selected from the group consisting of primary graft dysfunction (PGD), cardiac allograft rejection and cardiac allograft vasculopathy.

In some embodiments, the use or method of treatment may be for a condition that occurs downstream of graft dysfunction and/or GVHD. Accordingly, by preventing and/or treating the upstream condition, one provides a use or method that prevents and/or treats the downstream condition.

The assessment of viability of a donor tissue and/or donor organ following transplantation depends on the tissue and/or organ. For example, the viability of the lung can be assessed based on the level of oxygenation achieved by the recipient following transplantation. Accordingly, an organ-specific assessment can be compared with the clinically accepted criteria for said organ-specific assessment. For example, oxygenation is an accepted standard for assessing lung function, so can be analysed in a recipient following lung transplantation and compared with relevant population data for the expected oxygenation for the subject, or in comparison to oxygenation achievable by the recipient prior to transplantation. Techniques for assessing organ function are known to the skilled person. In some embodiments, the assessment of viability may be characterised by improved organ graft function in the long term (e.g. 1, 2, 3, 4, 5, 6, 7, 8, 9, 10, 11 or 12 months post-transplantation) compared with organ function within one week following transplantation of the subject, or to a control subject having undergone a tissue and/or organ transplant and experiencing delayed graft function who has not been exposed to isolated TAF MSCs. Alternatively, or additionally, the control for comparison may be a tissue and/or organ that has not been exposed to isolated TAF MSCs. Viability may also be referred to as preservation.

By “conditioning media” we refer to a media comprising isolated TAF MSCs that is suitable for conditioning a donor tissue and/or donor organ. A conditioning media may be used in a donor prior to removal of a tissue and/or organ, in a separate vessel in which the donor tissue and/or donor organ is stored (e.g. an EVLP chamber), or both. By “conditioning” we include the meaning that the media acts upon a tissue and/or organ in a way that retains, restores and/or rejuvenates the tissue and/or organ to a state closer to being physiologically healthy. Alternatively, or additionally, “conditioning” may refer to the retention, restoration and/or rejuvenation of a tissue and/or organ to parameters that would pass a criteria for said tissue and/or organ being deemed suitable for transplantation. Transplantation criteria for a tissue and/or organ, which varies depending on the tissue and/or organ, are known to the skilled person.

By “perfusion fluid” we refer to a fluid that is suitable for use during perfusion. Types of perfusion fluid are known in the art and vary depending on the perfusion technique, i.e. the perfusion fluid may be one that is suitable for use in perfusing a specific tissue and/or organ. Accordingly, the perfusion fluid can be any known perfusion fluid for use in perfusing a tissue and/or organ of interest, wherein the perfusion fluid further comprising isolated TAF MSCs. Use of isolated TAF MSCs in perfusion fluid may be in addition to or replacement of isolated TAF MSCs being present in a preceding and/or foregoing conditioning media. In some embodiments, the perfusion fluid is comprised of the same components as the conditioning media. In some embodiments, the perfusion fluid is comprised of different components as the conditioning media. Preferably, the isolated TAF MSCs used in the perfusion fluid are the same as those used in the conditioning media.

By “injection fluid” we refer to a fluid that is suitable for being injected into a tissue and/or organ. The injection fluid may be for use prior to, during and/or after transplantation of a donor tissue and/or donor organ. Use of isolated TAF MSCs in injection fluid may be in addition to or replacement of isolated TAF MSCs being present in a preceding and/or foregoing conditioning media and/or perfusion fluid. In some embodiments, the injection fluid is comprised of the same components as the conditioning media. In some embodiments, the injection fluid is comprised of different components as the conditioning media. In some embodiments, the injection fluid is comprised of the same components as the perfusion fluid. In some embodiments, the injection fluid is comprised of different components as the perfusion fluid. Preferably, the isolated TAF MSCs used in the injection fluid are the same as those used in the conditioning media and/or perfusion fluid.

Accordingly, the terms “conditioning media”, “perfusion fluid”, and “injection fluid” are used herein interchangeably. Therefore, any component referred to with respect to one of these terms is equally applicable for inclusion in a composition referred to by another of these terms.

In some embodiments, the conditioning media further comprises at least one antibiotic, vitamin, prostaglandin, bicarbonate and/or anticoagulant (e.g. heparin).

In some embodiments, the perfusion fluid further comprises at least one antibiotic, vitamin, prostaglandin, bicarbonate and/or anticoagulant (e.g. heparin).

In some embodiments, the injection fluid further comprises at least one antibiotic, vitamin, prostaglandin, bicarbonate and/or anticoagulant (e.g. heparin). As used herein, the terms “treat”, “treatment”, “treating”, or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilised (i.e., not worsening) state of allograft function (e.g. lung allograft), delay or slowing of organ function, and amelioration or palliation of symptoms associated with end stage organ disease.

A donor or recipient may be referred to as a subject. As used herein, a “subject” means a human or animal. Usually, the animal is a vertebrate such as a primate, rodent, domestic animal, or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include pigs, cows, horses, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, pig, mouse, rat, dog, cat, horse, or cow, but are not limited to these examples. In an embodiment, the subject is human. In addition, the methods described herein can be used to treat domesticated animals and/or pets.

A subject can be one who has been previously diagnosed with or identified as suffering from or having a condition in need of treatment (e.g., lung failure) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition, or one or more complications related to the condition or a subject who does not exhibit risk factors. For example, a subject can be one who exhibits one or more symptoms for a condition, or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

A therapeutically or prophylactically significant reduction in a symptom is, e.g., at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering isolated TAF MSCs. Measured or measurable parameters include clinically detectable markers of disease, for example, elevated or depressed levels of a biological marker, as well as parameters related to a clinically accepted scale of symptoms or markers for fibrosis and/or inflammation. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

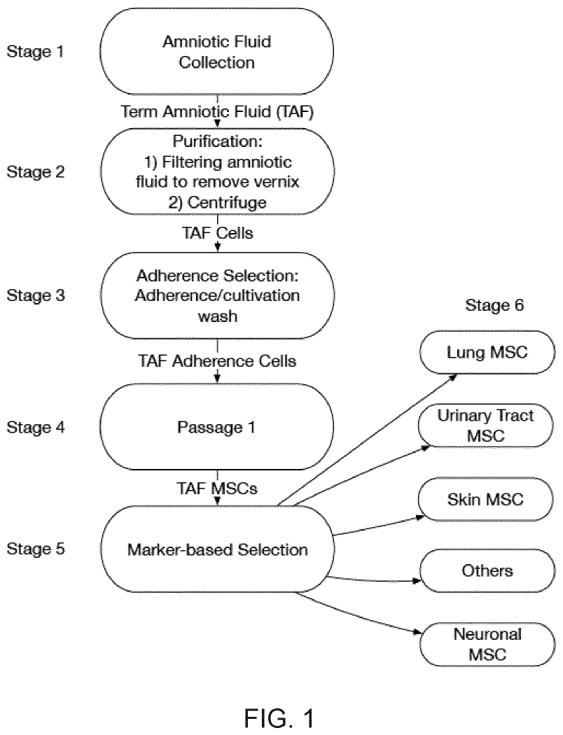

Methods of purifying, culturing and selecting MSC subpopulations with neonatal quality and adult tissue specificity are summarized in FIG. 1 and described in detail below. Examples disclosed herein relate to apparatuses and methods for collecting, purifying, isolating, expanding, differentiating, and maturing amniotic fluid-derived cells. The examples disclosed herein are not limited to collection of a certain type of amniotic-derived cell and the technologies disclosed herein are broadly applicable to different cells and tissues.

### Amniotic Fluid Collection

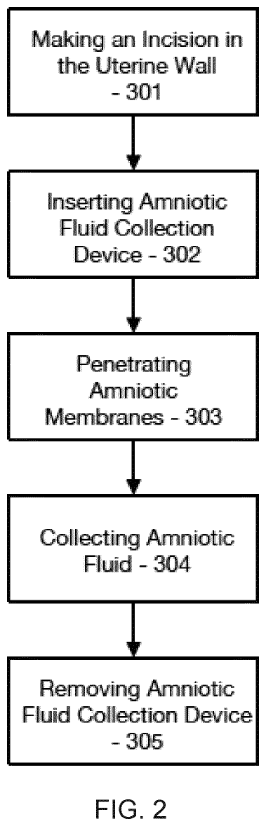

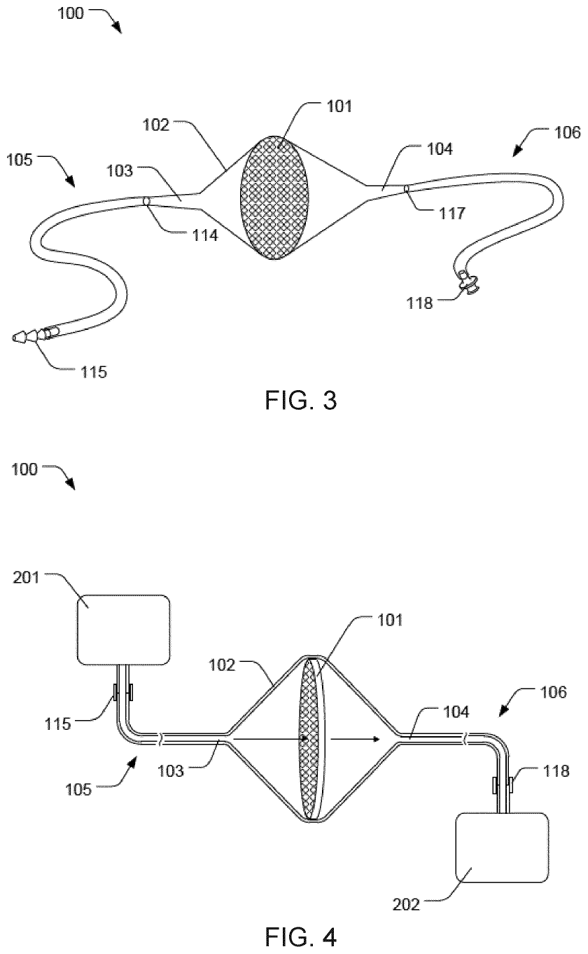

Amniotic fluid may be collected to produce term amniotic fluid (TAF) according to the methods described in U.S. patent application Ser. No. 14/776,499 (corresponding to US2016/0030489), the entire content of which is Specifically, FIG. 2 is a block diagram of an incorporated by reference. example of a method 300 of amniotic fluid collection, according to an exemplary example of the invention. It should be appreciated that method 300 may include any number of additional or alternative tasks. The tasks shown in FIG. 3 need not be performed in the illustrated order, and method 300 may be incorporated into a more comprehensive procedure or process having additional functionality not described in detail herein.

As shown in FIG. 2, method 300 may include making an incision in the uterine wall 301 of a pregnant mother, for example, during caesarean section. Step 301 may be performed with a standard physician's scalpel. As also shown in FIG. 2, method 300 may include inserting an amniotic fluid collector 302 through the incision in the uterine wall made in Step 301. Method 300 also includes penetrating the amniotic membrane 303 using the amniotic fluid collector of Step 302. Step 303 may also include penetrating the chorionic membrane. In one aspect, the tip is inserted to a 10 cm depth. In some examples, the tip is inserted to a depth of about 3 cm to about 30 cm. In some examples, the tip is inserted to a depth of about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, or about 29 cm.

Method 300 further includes collecting the amniotic fluid 304 from the amniotic sac using the amniotic fluid collector of Step 302. Step 304 may include initiating a siphon to transfer the amniotic fluid to a collection chamber of the amniotic fluid collector, such as by opening an inlet valve of the amniotic fluid collector. Step 304 may also include positioning a collection chamber of the amniotic fluid collector below an inlet of the amniotic fluid collector. Step 304 may also include coupling a negative pressure source to an outlet of the amniotic fluid collector to initiate transfer of the amniotic fluid. Step 304 may include relocating an inlet of the amniotic fluid collector to retrieve substantially all of the available amniotic fluid.

Finally, method 300 includes removing the amniotic fluid collector 305 from the amniotic sac. Step 305 may include closing an inlet valve of the amniotic fluid collector. In one example, no blood is visible in the collected material. Step 305 may also include emptying the collection system for further use/processing and sterilizing the exterior of the entire device. In one example, the exterior is sterilized using 70% ethanol so that the sterility may be maintained in any post-processing steps, such as in a laminar air flow bench setup, e.g., for isolation of cell material according to the present invention, and for fluid storage.

In one example, the amniotic fluid collection procedure is performed in less than one minute. In one example, the amniotic fluid collection procedure is performed in one to two minutes. In one example, the amniotic fluid collection procedure is performed in not more than three minutes. In one example, the method is simplified compared to standard operating procedures for cesarean sections, for example, by preventing spillage of the amniotic fluid into the operating wound, improving visibility and physical access. In one example, fetal skin is unaffected by the device tip.

### Purification

Term amniotic fluid (TAF) is purified by filtering term amniotic fluid to remove vernix. Although the term ‘term amniotic fluid’ is employed here and elsewhere in the present disclosure, it is understood that methods, processes, and devices of the present disclosure may be applied to all amniotic fluids and not just term amniotic fluid. Term amniotic fluid may be amniotic fluid collected at term caesarean section deliveries using, for example, a closed catheter-based system. For the purposes of the present description, ‘term amniotic fluid’ may be amniotic fluid collected at planned cesarean sections after 37 completed weeks of pregnancy or later, or at planned cesarean section close to term, for example after 36 completed weeks of pregnancy. Preferably, term amniotic fluid is taken at planned caesarean sections during week 37 of pregnancy or later.

FIG. 3 is a schematic illustration of an apparatus 100 for filtering amniotic fluid according to one example. The amniotic fluid contains amniotic cells originating from the fetus or the amniotic sac such as Mesenchymal stem cells. The amniotic fluid also contains other materials chafed off the skin such as hair and vernix. Material other than the amniotic cells are here referred to as particulate matter and may also comprise meconium, blood clots, etc. Particulate matter may be considered as anything larger than 20 μm. For the purposes of filtering, it may be particularly advantageous to treat anything larger than 30 μm or even 50 μm as particulate matter. Optionally, anything larger than the targeted amniotic cells may be treated as particulate matter. The amniotic fluid thus generally contains a mixture of amniotic cells and particulate matter. The apparatus 100 comprises a filter 101 for filtering the particulate matter from the amniotic fluid, and a chamber 102 enclosing the filter 101. The chamber 102 comprises a fluid inlet 103 and a fluid outlet 104. The chamber 102 enclosing the filter 101 should be construed as the filter 101 being isolated by the chamber towards the environment surrounding the chamber 102 such that there is no fluid communication between the amniotic fluid in the chamber 102 with said environment. Fluid communication through the chamber 102 is thus controlled via the fluid inlet 103 and the fluid outlet 104 in the example of FIG. 3. The filter 101 is attached to the inside of the chamber 102 between the fluid inlet 103 and the fluid outlet 104. FIG. 12 shows an example of a cross-section A-A as indicated in FIG. 12 of a circular chamber 102 and filter 101. It should however be understood that the chamber 102 and filter 101 may have varying shapes for optimization to different applications. The apparatus 100 comprises an inlet connector 105 arranged to form a sealing connection between the fluid inlet 103 and an amniotic fluid sample source 201 (shown in FIG. 4). FIG. 4 shows a schematic example of such source 201 of amniotic fluid. Having an inlet connector 105 connected to the fluid inlet 103 and configured to provide a sealing connection between the fluid inlet 103 directly to a source 201 of amniotic fluid provides for minimizing exposure to contaminants and an efficient aseptic handling of the amniotic fluid. This facilitates obtaining amniotic cells which allows post-filtration processing at an improved quality standard. Hence, an aseptic pharmaceutical production process is facilitated. The preparation of e.g. surfactant molecules may be facilitated. The apparatus 100 provides for improving the functioning of the amniotic stem cells, such as an improved engraftment phase following transplantation. Such improved processes are enabled by having the filter 101 enclosed in a chamber 102 and an inlet connector 105 arranged to form a sealing connection between the fluid inlet 103 of the chamber 102 and an amniotic fluid sample source 201. The risk of exposing the amniotic stem cells to contaminants, such as bacteria and viruses, is thus reduced. Exposure to oxygen is also minimized, which provides for reducing formation of oxygen free radicals which may negatively impact the functioning of the stem cells.

FIG. 3 shows an example where the inlet connector 105 comprises a tube 105 connected to the fluid inlet 103 at a first sealing connection 114. The inlet connector 105 may form a sealing connection with the fluid inlet 103 with a force-fitting connection, an adhesive, a clamp, or other fixation elements. In another example, such as schematically shown in FIG. 4, the inlet connector 105 is a continuous extension of the fluid inlet 103, without a separate fixation element, e.g. by being formed as a single piece by molding or other material forming techniques. FIGS. 3 and 4 show a second connector 115 configured to form a sealing connection with a sample source 201, such as a container or bag 201 containing amniotic fluid. The second connector 115 may comprise releasable force-fitting connection, a clamp, or a combination thereof, or other releasable fixation elements. The chamber 102, filter 101, fluid inlet 103, fluid outlet 104, and inlet connector 105 may be provided as a kit in a sterile packaging, e.g. as a disposable kit. Such kit, i.e. apparatus 100, thus provides for a facilitated and improved process of filtering and obtaining amniotic stem cells. Hence, in use, the amniotic fluid passes the filter 101 when flowing from the fluid inlet 103 to the fluid outlet 104. The particulate matter is thus deposited on the filter 101 and the amniotic fluid containing the amniotic cells flows through the fluid outlet 104. As seen in the example in FIG. 12, the filter 101 may be connected around its periphery 116 to the inner wall 113 of the chamber 102. This avoids passing of amniotic fluid from the inlet 103 to the outlet 104 without being filtered. The filter 101 may be tensioned or otherwise supported so that a folding or curving of the filter 101 in the chamber 102 is avoided. This maintains a defined mesh or pore size across the area of the filter 101 and thus defined filtering characteristics. Maintaining a defined mesh or pore size also reduces the risk of clogging the filter 101. Long-term performance may accordingly be improved.

The apparatus 100 may comprise an outlet 5 connector 106 to form a sealing connection between the outlet and an amniotic cell-receiving device 202, such as a centrifuge or other amniotic cell-processing equipment downstream of the apparatus 100. FIG. 4 shows a schematic example of such device 202. This minimizes exposure to contaminants and allows efficient aseptic handling of the amniotic fluid in post-filtering processing steps. FIG. 3 shows an example where the outlet connector 106 comprises a tube 106 connected to the fluid outlet 104 at a first sealing connection 117. The outlet connector 106 may form a sealing connection with the fluid outlet 104 with a force-fitting connection, an adhesive, a clamp, or other fixation elements. In another example, such as schematically shown in FIG. 4, the outlet connector 106 is a continuous extension of the fluid outlet 104, without a separate fixation element, e.g. by being formed as a single piece by molding or other material forming techniques. FIGS. 3 and 4 show a second connector 118 configured to form a sealing connection with an amniotic cell-processing device downstream of the apparatus 100, such as a centrifuge 202. The second connector 118 may comprise a force-fitting connection, a clamp, a combination thereof, or other releasable fixation elements. The connection between the second connector 118 and e.g. a centrifuge 202 may thus be repeatedly connected and disconnected, and also re-sealable to maintain a sealing connection in such procedure. The chamber 102, filter 101, fluid inlet 103, fluid outlet 104, inlet connector 105, and outlet connector 106 may be provided as a kit in a sterile packaging, e.g. as a disposable kit. Such kit, i.e. apparatus 100, thus provides a facilitated and improved process of filtering and processing of amniotic stem cells. The apparatus 100 may comprise a pump 122, 123, arranged to pressurize the amniotic fluid to flow from the fluid inlet 103 to the fluid outlet 104. This provides for a more effective filtering of the amniotic fluid. Larger volumes may be filtered in less time.

FIG. 6 shows an example where a pump 122 is connected to the fluid outlet 104 to draw amniotic fluid through the filter 101 in the direction of the indicated arrows. The pump 122 may be arranged at the fluid inlet 103 to push the amniotic fluid through the filter 101. The pump 122 may be a compact manually operated pump integrated with the fluid inlet 103, fluid outlet 104, inlet connector 105, or outlet connector 106.

FIG. 7 shows another example, described in more detail below, where a pump 123 is arranged to pressurize the amniotic fluid to flow from the fluid inlet 103 to the fluid outlet 104. The chamber 102 may comprise a conduit 119 arranged between the fluid inlet 103 and the fluid outlet 104. The pressure in the chamber 102 may be variable in response to fluid and/or gaseous communication through the conduit 119. The flow of amniotic fluid through the filter 101 may thus be optimized depending on the application, e.g. the flow rate through the filter 101 may be increased or decreased by varying the pressure in the chamber 102 via conduit 119.

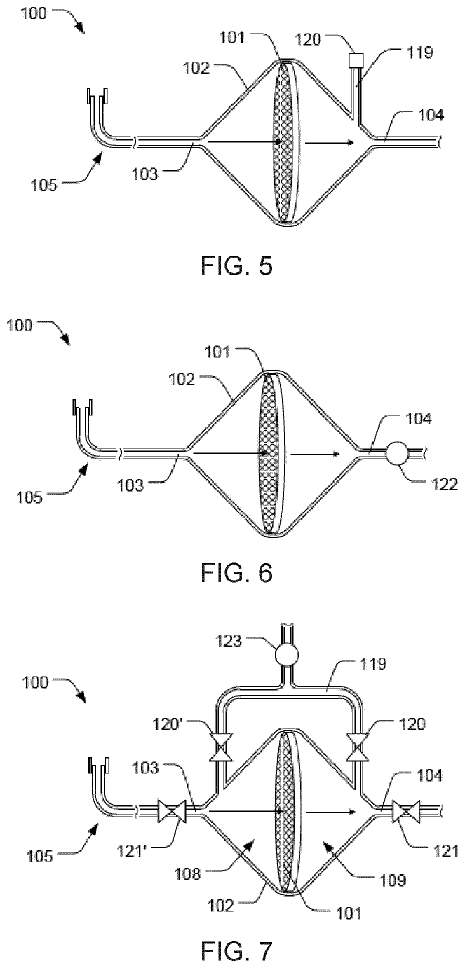

FIG. 5 shows an example in which a conduit 119 is in communication with the chamber 102. An access port 120, such as a connector or valve element, may be actuated to allow a fluid or gas to be expelled from the chamber 102, and/or injected into the chamber 102, to affect the pressure therein. The conduit 119 is arranged between the fluid outlet 103 and the filter 101 in FIG. 5, but the conduit 119 may be arranged between the fluid inlet 103 and the filter 101 in another example. FIG. 5 as described below shows a further example of a conduit 119 in communication with the chamber 102. A pump 123 may be arranged in communication with the conduit 119, as exemplified in FIG. 7. This facilitates optimization of the flow in the chamber 102 and the associated filtering process. In the example of FIG. 7 the conduit 119 is in variable communication with an upstream cavity 108 of the chamber 102 and a downstream cavity 109 of the chamber 102, i.e. the filter 101 may be arranged to divide the chamber 102 into an upstream cavity 108 and a downstream cavity 109. In FIG. 7 the conduit 119 is connected to both the upstream cavity 108 and the downstream cavity 109. The pump 123 is arranged to pressurize the amniotic fluid to flow from the upstream cavity 108 to the downstream cavity 109, or to flow from the downstream cavity 109 to the upstream cavity 108. The latter case may be advantageous in a situation in which a momentary reversed flow is desired, e.g. to clear out clogging or occlusion of the filter 101. In such case, valves 120, 120′, 121, 121′, as schematically indicated in FIG. 7 are operated to provide the desired flow directions. E.g. for a reversed flow, valves 120 and 121′ may be open and valves 120′ and 121 may be closed. Valves 121, 121′, may be open and\valves 120, 120′, may be closed in a normal filtering mode. The upstream cavity 108 may be pressurized by also opening valve 120′ in such filtering mode.

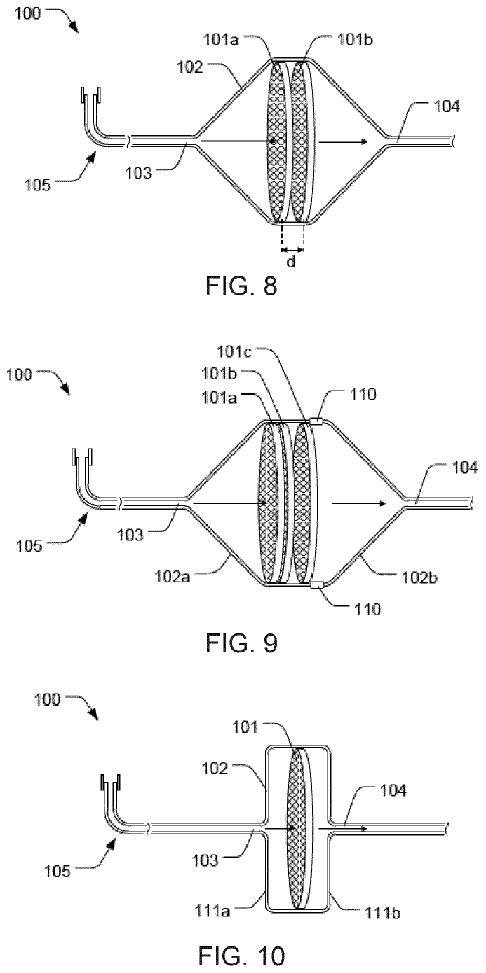

The filter 101 may comprise a first filter element 101a and a second filter element 101b arranged between the first filter element 101a and the fluid outlet 104, as schematically shown in FIG. 8. The second filter element 101b may have a mesh or pore size which is smaller than a mesh or pore size of the first filter element 101a. This allows effective filtering of particulate matter of gradually smaller dimensions. The risk of filter occlusion is thus reduced. This allows for a more reliable and robust filtering process of the amniotic fluid. An improved filtering of amniotic fluid containing a greater range in the size of particulate matter is also provided. Further, a larger fraction of the stem cells in the amniotic fluid may be obtained since the stem cells are not lost in clogged pores. Although FIG. 8 two filter elements 101a, 101b, it should be understood that any plurality of filter elements may be arranged in sequence in the chamber 102, with gradually decreasing mesh or pore size, in the direction of fluid flow from the fluid inlet 103 to the fluid outlet 104, for an effective filtering of particulate matter of gradually decreasing dimensions. The first and second filter elements 101a, 101b, may be separated by a distance (d) along a direction amniotic fluid flow from the fluid inlet 103 to the fluid outlet 104, as schematically indicted in the example of FIG. 8. The motion of the amniotic fluid between the first and second filter elements 101a, 101b, which in some case may involve turbid flow, may provide for further reducing the risk of unwanted build-up of particles on the first and second filter elements 101a, 101b.

The filter 101 may comprise a mesh having a mesh size in the range of 20-2000 μm. In another example, the filter 101 comprises a mesh having a mesh size in the range of 100-500 μm. This allows particularly effective filtration of particulate matter from the amniotic fluid. Turning again to FIG. 8, the first filter element 101a may comprise a mesh having a mesh size in the range of 500-1000 μm, and the second filter element 101b may comprise a mesh having a mesh size in the range of 30-150 μm. The first filter element 101a may thus remove larger debris, followed by removal of smaller particles with the second filter element 101b. This allows a particularly effective filtering of particulate matter of varying size and reliable filtering of increased volumes over longer time periods since the risk of clogging is further minimized. As previously mentioned, any plurality of filter elements may be arranged in succession in the chamber 102.

FIG. 9 shows three filter elements 101a, 101b, 101c, arranged in the chamber 102. In some examples the filter element having the smallest mesh or pore size, arranged furthest downstream in the chamber 102 may, such as filter element 101b in FIG. 6 and filter element 101c in FIG. 9, may have a mesh or pore size dimensioned so that only single amniotic cells or amniotic cell clumps smaller than 10 cells pass through the filter 101. The smallest mesh or pore size in such an example may be approximately 30 μm. The filter 101 may comprise a mesh such as a nylon mesh. The filter 101 may comprise a porous material having a variable pore size through the filter 101 in the direction of flow of the amniotic fluid from the fluid inlet 103 to the fluid outlet 104. I.e. larger debris is removed at the surface of the filter 101 closest to the inlet 103 whereas particles of smaller size are removed deeper into the filter, as the amniotic fluid flows through the filter 101 in a direction towards the outlet 104 and the size of the pores get smaller. As previously mentioned, the chamber 102 may comprise an upstream cavity 108 and a downstream cavity 109. The upstream and downstream cavities 108, 109, may be formed as an integrated piece to form the chamber 102, e.g. in a molding process or by other material forming techniques. The upstream and downstream cavities 108, 109, may be formed as separate units which are then connected to each other to form a sealing connection, e.g. by an adhesive or by welding. The filter 101 may be attached simultaneously or subsequently with such welding process or by the aforementioned adhesive.

The upstream and downstream cavities 108, 109, may be releasably connectable to each other at a connecting element 110, to form a sealing connection, as schematically shown in FIG. 9. This allows opening of the chamber 102, e.g. for replacing the filter 101. The filter 101 may thus be releasably connectable to the chamber 102, e.g. filter elements 101a, 101b, 101c, may be releasably connectable to the chamber 102 in FIG. 7. This allows facilitated customization to different applications since filter elements 101a, 101b, 101c, of different pore or mesh size, or different number of such filter elements may be mounted in the chamber 102.

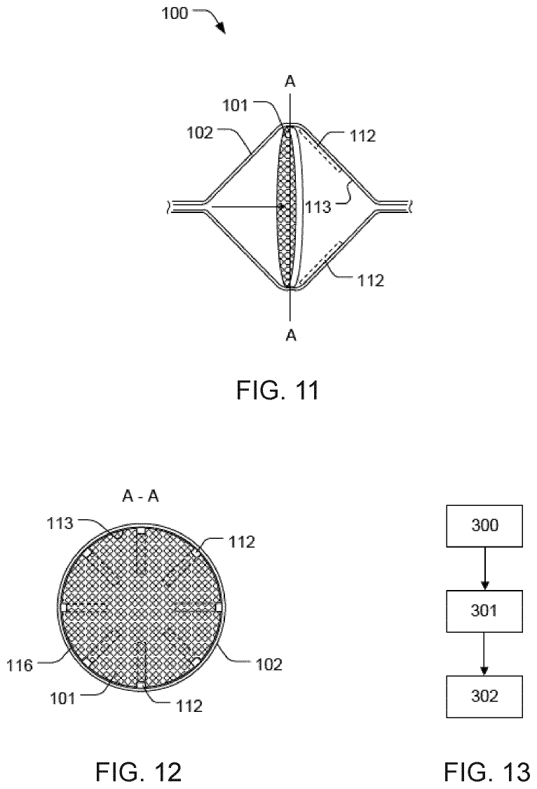

The connecting element 110 is configured to form a sealing connection upstream and downstream cavities 108, 109, and may comprise an annular gasket extending around the periphery of the upstream and downstream cavities 108, 109. The filter 101 may comprise a cartridge of different numbers of filter elements 101a, 101b, 101c, with different pore sizes that could be tailored to the particular amniotic fluid sample. For example, evaluation of the amniotic fluid turbidity and degree of milkiness (level of vernix both in particle size and opaqueness) could be an indicator of the appropriate filter cartridge to use. An accompanying chart for which to compare the amniotic fluid sample with could indicate which filter cartridge to use. The upstream cavity 108 and/or the downstream cavity 109 may be funnel shaped. FIGS. 3-9 show examples where both the upstream and downstream cavities 108, 109, are funnel shaped. FIG. 11 shows an example where only the downstream cavity 109 is funnel shaped. Having a funnel shape may be advantageous for directing the flow of amniotic fluid along a desired vector of symmetry through the filter 101 and apparatus 100. The upstream cavity 108 and/or the downstream cavity 109 may comprise a chamber wall 111a, 111b being arranged essentially in parallel with the filter 101, i.e. perpendicular to the direction of flow of the amniotic fluid from the fluid inlet 103 to the fluid outlet 104. FIG. 10 shows an example where chamber walls 111a, 111b, of the upstream and downstream cavities 108, 109 are arranged essentially in parallel with the filter 101. This minimizes the space inside the chamber 102, while maintaining adequate filter area, to minimize the risk of introducing e.g. air that may disturb surfactant molecules, reduce the risk of infection, and reduce detrimental formation of reactive oxygen species in the amniotic cells. The chamber 102, and/or the inlet connector 105, and/or the outlet connector 106 may be formed from a phthalate free PVC material. This provides for an apparatus which is suitable to be in contact with pharmaceutical starting materials such as amniotic cells.

The apparatus 100 may comprise protrusions 112 arranged to extend from an inner wall 113 of the chamber 102. FIGS. 11 and 12 show examples of such protrusions 112, in a cross-sectional side view and through cross-section A-A respectively. The protrusions 112 provides support for the filter 101 in case the filter 101 would start bend and fold towards the inner wall 113. Thus, a flow through the mesh or pores of the filter 101 is still possible in such case since the filter 101 may be supported by the protrusions 112 at a distance from the inner wall 113, i.e. the protrusions 112 allows for further limiting the risk of flow restriction and provides for an efficient, robust and reliable filtering.

FIG. 13 is a flow chart of a method 300 of filtering amniotic fluid containing particulate matter and amniotic cells. The method 300 comprises forming 301 a sealing connection between a fluid inlet 103 of a chamber 102 and an amniotic fluid sample source 201. The method 300 comprises passing 302 the amniotic fluid through a filter 101 enclosed in the chamber 102 by providing a flow of the amniotic fluid from the fluid inlet 103 to a fluid outlet 104 of the chamber 102. Particulate matter is thereby deposited on the filter 101 and the amniotic fluid containing amniotic cells flows through the outlet 104. The method 300 thus provides for the advantageous benefits as described in relation to apparatus 100 and FIGS. 3-12 above. The method 300 provides for effective and sterile filtration of the amniotic fluid to obtain amniotic cell samples of high quality.

In one embodiment, removing particulate material from the TAF to obtain purified TAF cells may be done by applying any known method in the art such as filtration, centrifugation, etc. The TAF may be filtered through a filter having a pore size at or above 20 μm. The filter may be made from any synthetic material including but not limited to cellulose acetate, cellulose nitrate (collodion), polyamide (nylon), polycarbonate, polypropylene and polytetrafluoroethylene (Teflon). In one embodiment removing particulate material is done by applying apparatus 100.

### Adherence Selection

Various terms known to one skilled in the art have been and will be used throughout the specification, for example, the terms “express, expression, and/or expressing” in the context of a cell surface marker are meant to indicate the presence of a particular marker on the surface of a cell, said surface marker having been produced by the cell. Surface marker expression may be used to select between different cell populations, for example, positively selecting for surface marker expression indicates the selection of a cell population that more strongly expresses a particular surface marker as compared to another cell population. Conversely, negatively selecting for cell surface marker expression indicates the selection of a cell population that more weakly expresses a particular surface marker as compared to another cell population.

As explained above and elsewhere in the specifications, TAF contains various progenitor cell types. In certain examples, particular progenitor cell types may be isolated and propagated via adherence selection. For example, a vitronectin substrate, Synthemax (Merck, CORNING®, Synthemax®, II-SC SUBSTRATE, CLS3535-1EA) may be used as a coating to create a more in vivo-like environment for stem cell culture, thereby limiting maturation of the TAF-derived progenitor cells and maintaining plasticity. Synthemax is an animal-component free, synthetic, flexible vitronectin-based peptide substrate for serum or serum-free expansion of human progenitor/stem cells and other adult stem cell types. One of skill in the art will understand that the vitronectin-based peptide substrate may include a portion of a vitronectin protein, such as a particular peptide sequence of vitronectin. Alternatively, intact vitronectin protein may be used. Synthemax vitronectin substrate offers a synthetic, xeno-free alternative to biological coatings and/or feeder cell layers commonly used in cell culture and known in the art. Briefly, standard tissue-culture treated flasks may be coated with about 0.2 mL Synthemax/cm² at 10 μg/mL giving a surface density of 2 μg/cm², and incubated at 37° C. for about 1 h, 1.5 h, 2 h, 4 h, 8 h, or more than 8 h or at room temperature for about 2 h, 1 h, 4 h, 8 h or more than 8 h with surplus solution optionally being removed and replaced. In certain examples, Synthemax may be coated at a surface density of about: 1 to 5 μg/cm², such as 2 μg/cm², 1 to 10 μg/cm², 1.5 to 4 μg/cm², 1 to 3 μg/cm², or about 1.5 to 2.5 g/cm².

In other embodiments, adherence selection can be performed using a surface coated with, for example, Collagen, Fibronectin. Alternatively, adherence selection can be performed using an uncoated surface comprising a tissue-culture treated plastic.

Cells purified from TAF fluid may be gently re-suspended in prewarmed xeno-free cell culture media, with the cell suspension is then added to the Synthemax-coated flasks. Media may be changed at various times after addition to the flasks, for example, after about: 2 h to 168 h, 12 h to 96 h, 24 h to 72 h, 36 h to 60 h, 42 h to 56 h, or 48 h, and then subsequently changed about: every day, every other day, every third day, every fifth day, once a week, once every two weeks or about less than once every two weeks. Through repeated removal of spent medium, the non-attached cells may be removed, thereby selecting the MSCs by their affinity for attachment to the Synthemax-treated surface. The cells may be cultured for a period of time, such as about, for example, 4 d, 7 d, 10 d, 11 d, 12 d, 13 d, 14 d, 18 d, 21 d, 28 d or longer than 21 d. Optionally, the cells may be cultured under hypoxic conditions: hypoxia priming may alter cell metabolism during expansion, increase resistance to oxidative stress, and thereby improve the engraftment, survival in ischemic microenvironments, and angiogenic potential of transplanted MSCs. After culturing, the PO colonies (Colony forming Units—CFUs) that have formed may be dissociated and pooled. After pooling, the remaining cells may be predominantly non-tissue specific MSCs. In certain examples, the pooled PO cells may be gently re-suspended in pre-warmed xeno-free cell culture media and re-plated on tissue-culture treated flasks without Synthemax for passaging. The pooled cells may be seeded at a seeding density of from between about: 100 to 10000 cells/cm², 500 to 8000 cells/cm², 1000 to 5000 cells/cm², or about 2000 to 4000 cells/cm². The media may be changed about every 1 d, 2 d, 4 d, or more than four days. After a period of time, such as about 2 d, 4 d, 7 d, or more than 7 d, the cells may be dissociated and harvested. Further selective MSC isolation may be achieved as described below.

### Identification of Markers

When comparing the genetic expression profiles of TAF-MSCs and adult-type MSCs derived from adipose tissue or bone marrow by RNAseq, TAF-MSCs tend to express more of some genes present in adult-type MSCs and less of others. Identification of both positive and negative TAF-MSC specific neonatal cell-surface markers can allow for sorting of the MSCs with neonatal quality from those that have differentiated further and are of less importance as progenitor cells using e.g. ligands such as antibodies and aptamers or other selection techniques.

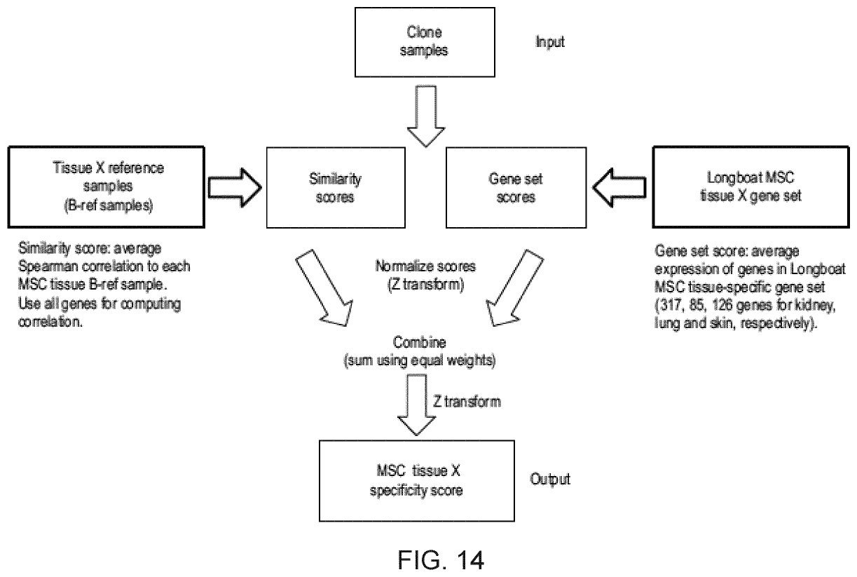

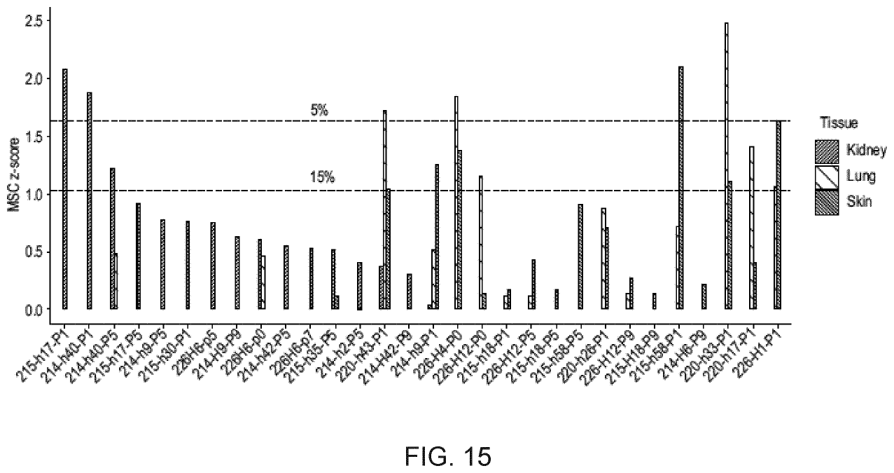

The cell surface markers distinguishing tissue relevant cells from other MSCs may be elucidated via a bioinformatics process utilizing a tissue-specificity score algorithm. An example of an MSC tissue-specificity score algorithm is shown in FIG. 14. Tissue-specificity may be measured as a combination of two components: a ‘tissue transcriptional similarity’ also known as a similarity score and a “tissue-specific gene expression program” also known as a gene set score. In certain examples, the similarity score may be an Average Spearman correlation to each MSC tissue reference sample (for example a fetal lung MSC sample). In examples, the gene set score may be the average expression of genes in a tissue-specific gene set. As shown in FIG. 14, in certain examples, after normalizing the similarity and gene set scores using a Z-transform to convert the input values, which is a sequence of real or complex numbers, into a complex frequency-domain representation, then combining them assigning equal weight to each score and transforming combined values using a Z-transform, the resulting output is an MSC tissue specificity score. The MSC tissue-specificity score measures the relative tissue-specificity among the input samples by measuring how many standard deviations a sample is more or less specific to a given tissue compared to the average input sample. For example, an MSC tissue-specificity score may indicate how much more a clone sample appears to have a tissue specific phenotype, such as a lung phenotype, as compared to an average clone. Such an approach allows for identification of the top X % percentile scores using a normal distribution function, effectively the top X % of clones that are most tissue-specific to the relevant tissue.

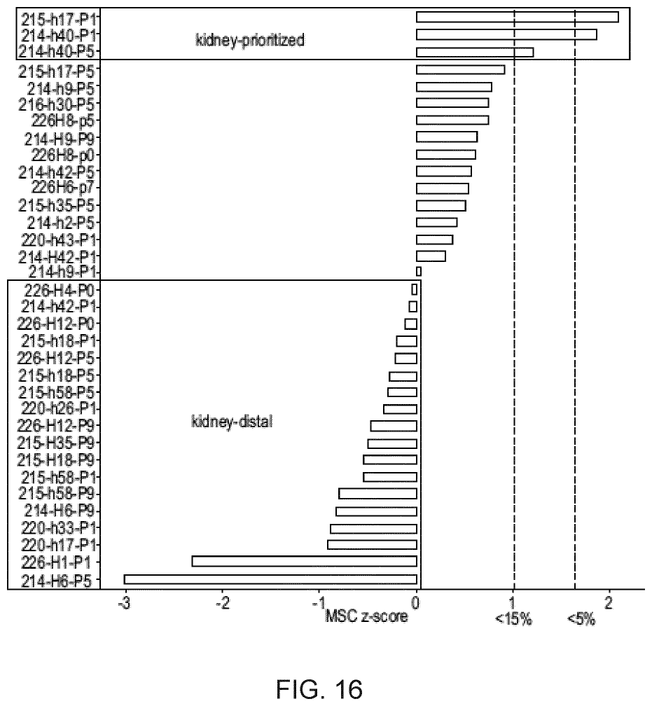

In one example, for a given tissue, tissue-prioritized clones can be defined as any clone belonging to the top X % percentile score, where X is any percentage within a range having a lower end from about 0.1 to 25, such as about 1, 5, 10, 15 and 20, and an upper end from about 30 to 75, such as about: 35, 40, 45, 50, 55, 60, 65 or 70. An example of TAF-MSC tissue-specificity prioritization results is shown in FIG. 21, in which thresholds at 15% and 5% are visible. Having prioritized tissue-specific clones, candidate surface marker genes may then be identified. For each tissue, two groups may be defined: tissue-prioritized and tissue-distal. A suitable analysis program may be used to make this determination, for example DEseq2 from Bioconductor.org. The tissue-prioritized group may include clones with a score in the top 15% percentile. The tissue-distal group may include clones in the bottom Y % percentile in which Y is any percentage within the range having a lower end from about 25 to 70, such as about: 30, 35, 40, 45, 50, 55, 60 or 65 and an upper end from 75 to 99.9, such as about: 80, 85, 90, 95 or 99. FIG. 16 shows an example of such analysis on kidney tissue. Next, differentially expressed genes between the tissue-prioritized and tissue-distal groups may be identified. Finally, the differential expression results may be annotated with surface marker gene information.

In certain examples, to identify tissue-specific cell surface markers, surface marker genes with a more than a Z-fold increase, where Z is at least about: 1.5-fold, 2-fold, 2.5-fold, 3-fold, 3.5-fold, 4-fold, 5-fold, 8-fold, 10-fold, 12-fold, 15-fold or even more-fold increase in expression (log 2FoldChange) in prioritized clones compared to an average clone and a Transcripts Per Kilobase Million (TPM) of more than about 500, such as more than about: 1000, 1500, 2000, 2500, 3000, 5000 or even higher may be selected to give the top tissue-specific marker candidates, such as approximately the top: 5, 10, 20, 30, 40, 50, 60, 70, 100 or more, for example such as those shown below in Tables 3-6 and further described in more detail below. Suitable log 2FoldChange and TPM values may vary even further depending on tissue type specificities depending on the abundance/absence of good markers.

Applying the tissue specificity algorithms described above to identify surface markers, after adhesion selection and passaging, the TAF-MSCs cells may express various identified surface markers as shown below in Table 1, indicative of non-tissue specific TAF MSCs. One of skill in the art will understand that such surface markers may be present at various surface densities and may be upregulated or downregulated in comparison to other cell types. Therefore, such surface markers may be used to identify and isolate particular cell types. In some instances, the surface markers listed in Table 1 below may be at least 8-fold more highly expressed for TAF MSCs on average compared to other MSC cell types, particularly as compared to adult MSCs derived from bone marrow or adipose tissue. The thresholds used to generate Table 1 are as follows: X was selected as 15%, Y was selected as 50%, Z was selected as 8-fold and a TPM of more 3000 was selected. One of skill in the art will understand that the numbering used in Table 1 and all tables herein is merely used to indicate a total number of identified markers and not to indicate that one particular marker is more strongly expressed and/or preferred compared to another marker.

TABLE 1
Group A markers.

| | | |
|---|---|---|
| 1. | TBC1D3K | TBC1 domain family member 3K |
| 2. | AIF1L | allograft inflammatory factor 1 like |
| 3. | CDHR1 | cadherin related family member 1 |
| 4. | NKAIN4 | sodium/potassium transporting ATPase interacting 4 |
| 5. | ABCB1 | ATP binding cassette subfamily B member 1 |
| 6. | PLVAP | plasmalemma vesicle associated protein |
| 7. | MSLN | mesothelin |
| 8. | L1CAM | L1 cell adhesion molecule |
| 9. | HAVCR1 | hepatitis A virus cellular receptor 1 |
| 10. | MAL2 | mal, T cell differentiation protein 2 (gene/ |
| | | pseudogene) |
| 11. | SLAMF7 | SLAM family member 7 |
| 12. | DOC2B | double C2 domain beta |
| 13. | ESAM | endothelial cell adhesion molecule |
| 14. | GABRB1 | gamma-aminobutyric acid type A receptor beta1 |
| | | subunit |
| 15. | CDH16 | cadherin 16 |
| 16. | IGSF3 | immunoglobulin superfamily member 3 |
| 17. | DSC3 | desmocollin 3 |
| 18. | RHEX | regulator of hemoglobinization and erythroid cell |
| | | expansion |
| 19. | KCNIP1 | potassium voltage-gated channel interacting protein 1 |
| 20. | CD70 | CD70 molecule |
| 21. | GFRA1 | GDNF family receptor alpha 1 |
| 22. | CRB3 | crumbs cell polarity complex component 3 |
| 23. | CLDN1 | claudin 1 |
| 24. | AC118754.1 | novel transcript |
| 25. | SCN5A | sodium voltage-gated channel alpha subunit 5 |
| 26. | FGFR4 | fibroblast growth factor receptor 4 |
| 27. | KCNK3 | potassium two pore domain channel subfamily K |
| | | member 3 |
| 28. | DYSF | dysferlin |
| 29. | EFNA1 | ephrin A1 |
| 30. | KCNJ16 | potassium inwardly rectifying channel subfamily J |
| | | member 16 |
| 31. | MARCHF1 | membrane associated ring-CH-type finger 1 |
| 32. | SYTL1 | synaptotagmin like 1 |
| 33. | CLSTN2 | calsyntenin 2 |
| 34. | ITGB4 | integrin subunit beta 4 |
| 35. | VAMP8 | vesicle associated membrane protein 8 |
| 36. | GPRC5C | G protein-coupled receptor class C group 5 member C |
| 37. | CD24 | CD24 molecule |
| 38. | CELSR2 | cadherin EGF LAG seven-pass G-type receptor 2 |
| 39. | CDH8 | cadherin 8 |
| 40. | GRIP1 | glutamate receptor interacting protein 1 |
| 41. | DMTN | dematin actin binding protein |
| 42. | F11R | F11 receptor |
| 43. | CADM1 | cell adhesion molecule 1 |
| 44. | CDH6 | cadherin 6 |
| 45. | F2RL2 | coagulation factor II thrombin receptor like 2 |
| 46. | LYPD1 | LY6/PLAUR domain containing 1 |
| 47. | SLC6A6 | solute carrier family 6 member 6 |
| 48. | DSG2 | desmoglein 2 |
| 49. | ADGRG1 | adhesion G protein-coupled receptor G1 |
| 50. | CCKAR | cholecystokinin A receptor |
| 51. | OXTR | oxytocin receptor |
| 52. | ITGA3 | integrin subunit alpha 3 |
| 53. | AMIGO2 | adhesion molecule with Ig like domain 2 |
| 54. | CELSR1 | cadherin EGF LAG seven-pass G-type receptor 1 |
| 55. | EPHB2 | EPH receptor B2 |

As will be understood by one of skill in the art, suitable combinations of the markers listed in Table 1 may be used to separate TAF-MSCs from adult MSCs by selecting for specific markers from Table 1 or combinations of two, three, four, five, six or more markers from Table 1. In certain examples, TAF MSCs can be more specifically identified by identifying a combination of stronger expression, such as 8-fold or more stronger expression of any combination of the foregoing markers, e.g., TBC1D3K and/or AIF1L and/or CDHR1 and/or NKAIN4 and/or ABCB1 and/or PLVAP as compared to adult MSCs. When using combinations of markers, identification may be achieved with a lower threshold of stronger expression, such as 2-fold or more, 4-fold or more, or 6-fold or more expression of each of the markers.

In contrast to the above surface markers that may be more strongly expressed on the surface of TAF-MSCs (positive markers) compared to adult MSCs, in certain examples, the below surface markers in Table 2 may be more weakly expressed on TAF-MSCs as compared to other cell types (negative markers), such as ⅛-fold or less expression (optionally with TPM threshold>500) of any combination of the foregoing markers versus adult MSCs: IL13RA2, CLU, TMEM119, CEMIP, and LSP1. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate TAF MSCs. In addition, those skilled in the art will also recognize that combinations including both negative and positive markers, such as at any of the thresholds described above, can also be effective to more specifically isolate TAF MSCs.

TABLE 2
Markers that have reduced expression in TAF MSCs.

| | | |
|---|---|---|
| 1. | IL13RA2 | Interleukin-13 receptor subunit alpha-2 |
| 2. | CLU | Clusterin |
| 3. | TMEM119 | Transmembrane Protein 119 |
| 4. | CEMIP | Cell Migration Inducing Hyaluronidase 1 |
| 5. | LSP1 | Lymphocyte Specific Protein 1 |
| 6. | GPNMB | Glycoprotein Nmb |
| 7. | FAP | Fibroblast Activation Protein Alpha |
| 8. | CRLF1 | Cytokine Receptor Like Factor 1 |
| 9. | MME | Membrane Metalloendopeptidase |
| 10. | CLMP | CXADR Like Membrane Protein |
| 11. | BGN | Biglycan |
| 12. | DDR2 | Discoidin Domain Receptor Tyrosine Kinase 2 |

Marker-Based Selection

Amniotic fluid contains heterogenous cells in a homogenous fluid. Hence, a marker-based selection may be needed. One example of marker-based selection is via the use of Fluorescence activated cell sorting (FACS). Fluorescence activated cell sorting (FACS) may be used to purify the cell population of TAF-MSCs, FACS allows for a very high purity of the desired cell population, even when the target cell type expresses very low levels of identifying markers and/or separation is needed based on differences in marker density. FACS allows the purification of individual cells based on size, granularity and fluorescence. As will be understood by one of skill in the art, FACS may be used to select for certain cell populations that express one cell surface marker more than another cell population and vice-versa. In some examples of methods of purification, bulk methods of purification such as panning, complement depletion and magnetic bead separation, may be used in combination with FACS or as an alternative to FACS. In brief, to purify cells of interest via FACS, they are first stained with fluorescently-tagged monoclonal antibodies (mAbs), which recognize specific surface markers on the desired cell population. Negative selection of unstained cells may also allow for separation. For GMP production of cells according to some examples, FACS may be run using a closed system sorting technology such as MACSQuant® Tyto®. Samples may be kept contamination-free within the disposable, fully closed MACSQuant Tyto Cartridge. Further, filtered air may drive cells through a microchannel into the microchip at very low pressure (<3 PSI). However, before entering the microchannel, potential cell aggregates may be held back by a filter system guaranteeing a smooth sorting process. The fluorescence detection system may detect cells of interest based on predetermined fluorescent parameters of the cells. Based on their fluorescent and scatter light signatures, target cells may be redirected by a sort valve located within the microchannel. For certain examples of methods of purification, the success of staining and thereby sorting may depend largely on the selection of the identifying markers and the choice of mAb. Sorting parameters may be adjusted depending on the requirement of purity and yield. Unlike on conventional droplet sorters, cells sorted by the MACSQuant Tyto may not experience high pressure or charge, and may not get decompressed. Therefore, such a gentle sorting approach may result in high viability and functionality of cells. Alternatively, other marker-based selection techniques may be known to the skilled person and employed here. These include, but are not limited to, Magnetic-activated cell sorting, Microfluidic based sorting, Buoyancy activated cell sorting, mass cytometry etc.

### Tissue Specific Cells and Usage

Lung TAF Cell Markers

As explained above, analysis of RNAseq data from TAF-MSC clones, adult and neonatal MSC reference material as well as fetal fibroblasts and publicly available expression datasets may be used to identify and characterize TAF-MSC cells. For example, sub-populations of TAF-MSCs may be established by clustering their expression data (RNAseq) with neonatal reference samples. Such sub-populations include, but are not limited to, lung MSC, urinary tract MSC (described also as kidney MSCs in the present disclosure), and skin MSC. Gene lists of highly and lowly expressed genes for each cluster of expression data may allow for identification of surface maker genes for each cluster. Using such data comparison, sub-populations of TAF cells were compared to adult MSC cells based on their gene expressions (RNAseq) resulting in a list of neonatal-specific surface marker genes for each cluster. A number of surface markers of interest associated with lung TAF cells were identified. For example, a non-exclusive list of preferred surface markers used to identify and separate lung TAF cells are provided below. Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of lung TAF cells from the general population of TAF-derived cells and/or TAF-MSC cells. In some examples, the below non-exclusive list of surface markers may be more highly expressed on the surface of Lung-TAF cells as compared to other cell types, such as other TAF-derived cells and/or TAF-MSC cells.

As explained above, bioinformatics techniques may be used to identify tissue-specific surface markers, therefore, the surface markers identified in Table 3 may have at least a 10-fold increase in expression on prioritized clones compared to the average TAF-MSC clone (optionally with TPM threshold>2000).

TABLE 3
Group B markers.

| | | |
|---|---|---|
| 1. | PCDH19 | protocadherin 19; |
| 2. | DDR1 | discoidin domain receptor tyrosine kinase 1 |
| 3. | MME | membrane metalloendopeptidase |
| 4. | IFITM10 | interferon induced transmembrane protein 10; |
| 5. | BGN | biglycan |
| 6. | NOTCH3 | notch receptor 3; |
| 7. | SULF1 | sulfatase 1; |
| 8. | TNFSF18 | TNF superfamily member 18; |
| 9. | BDKRB1 | bradykinin receptor B1; |
| 10. | FLT1 | fms related tyrosine kinase 1 |
| 11. | PDGFRA | platelet derived growth factor receptor alpha; |
| 12. | TNFSF4 | TNF superfamily member 4; |
| 13. | UNC5B | unc-5 netrin receptor B; |
| 14. | FAP | fibroblast activation protein alpha |
| 15. | CASP1 | caspase 1; |
| 16. | CD248 | Endosialin; |
| 17. | DDR2 | discoidin domain receptor tyrosine kinase 2 |
| 18. | PCDH18 | protocadherin 18; and/or |
| 19. | CRLF1 | cytokine receptor like factor 1; |

In contrast to the above surface markers that may be more strongly expressed on the surface of lung TAF MSCs, in certain examples, the below surface markers may be more weakly expressed on lung TAF MSCs as compared to other cell types, such as other TAF-derived cells and/or TAF-MSCs: CD24, ITGB4, TNFSF10, GFRA1, CD74, FGFR4, HAVCR1, and OSCAR. As will be understood by one of skill in the art, one, two, three, four, or more of the aforementioned more weakly expressed surface markers may be used to separate lung TAF cells from other cell types such as other TAF-derived cells and/or TAF-MSCs.

In certain examples, the cell surface marker CD248 (Endosialin) may be used to sort lung TAF MSCs from a population of TAF MSCs. Further surface markers that may be used to sort lung TAF MSCs include DDR-1 (discoidin domain receptor tyrosine kinase 1) as well as LRRC38 (Leucine Rich Repeat Containing Protein 38), all three of which have been identified via antibodies as useful markers for separation. In some examples, Endosialin, DDR-1, and/or LRRC38 alone or in combination with other markers may be used to sort. Endosialin may be combined with DDR-1 or LRRC38 to sort, or DDR-1 and LRRC38 may be combined without Endosialin.

As will be understood by one of skill in the art, suitable combinations of the markers listed in Table 3 and CD248, DDR-1, and LRR38 may be used to separate lung TAF MSCs from TAF MSCs by selecting for specific markers from Table 3 or combinations of two, three, four, five, six or more markers from Table 3 and/or CD248 and/or DDR-1 and/or LRR38. In certain examples, lung TAF MSCs can be more specifically identified by identifying a combination of stronger expression, such as 10-fold or more stronger expression (optionally with TPM threshold>2000) of any combination of the foregoing markers, e.g., PCDH19 and/or DDR1 and/or MME and/or IFITM10 and/or BGN and/or NOTCH3 and/or CD248 and/or DDR-1 and/or LRR38 as compared to TAF MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of lung TAF MSCs (positive markers) compared to TAF MSCs, in certain examples, the below surface markers may be more weakly expressed on lung TAF-MSCs as compared to other cell types (negative markers), such as ⅛-fold or less expression (optionally with TPM>500) of any combination of the foregoing markers versus TAF MSCs: CD24, ITGB4, TNFSF10, GFRA1, CD74, FGFR4, HAVCR1, and OSCAR. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate lung TAF MSCs. In addition, those skilled in the art will also recognize that combinations including both negative and positive markers, such as at any of the thresholds described above, can also be effective to more specifically isolate lung TAF MSCs.

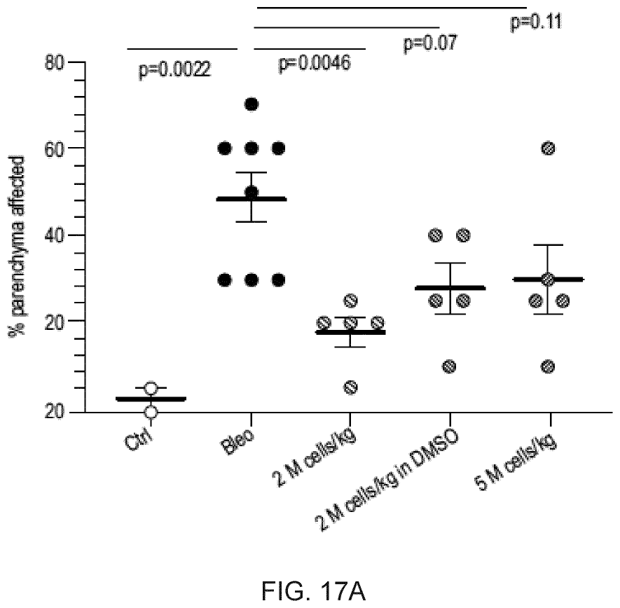

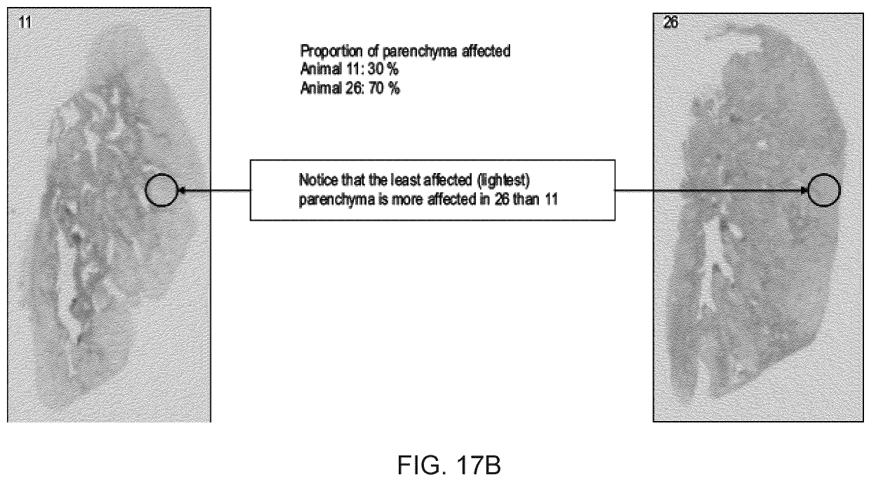

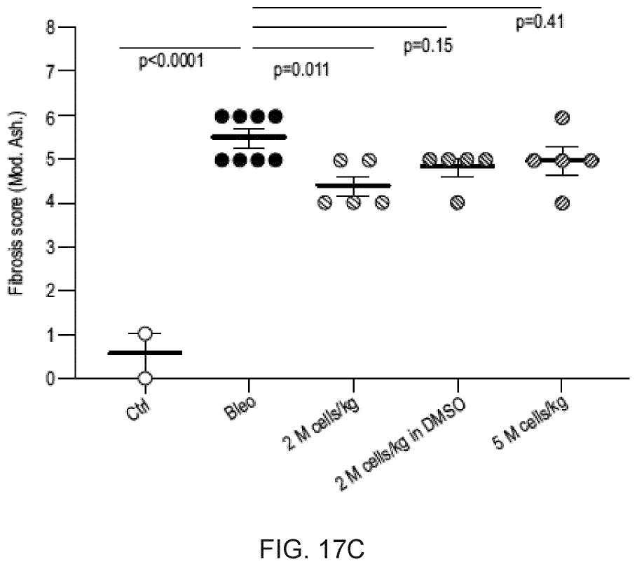

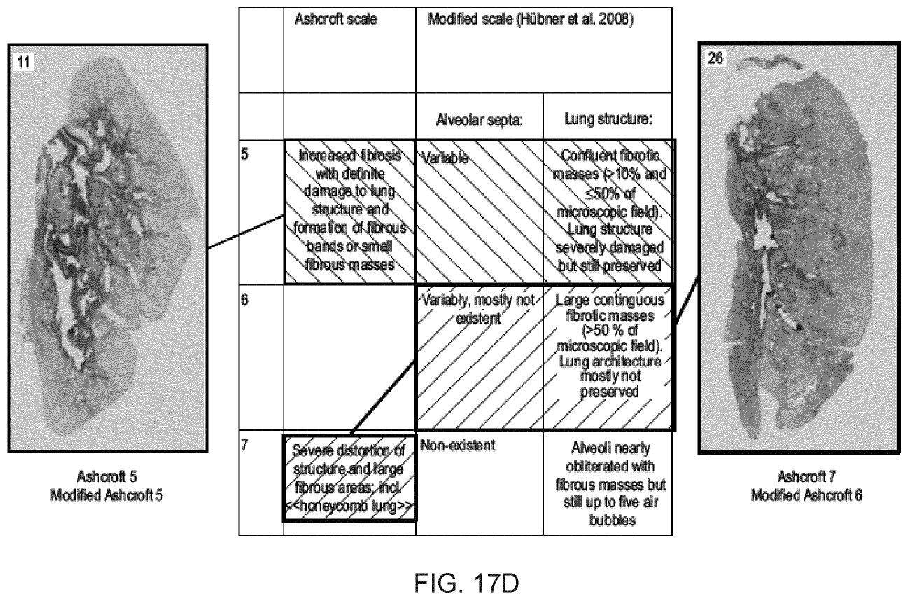

FIGS. 17A-17D show an example of the results from a proof-of-principle study on the potential use of Lung TAF MSCs for treatment, performed using neonatally sorted TAF MSCs expressing MSC lung cell surface markers including CD248, DDR1, and LRRC38 (called “LBX-THX-001 cells”). The purpose of the study was to investigate the effects of LBX-THX-001 cells in a bleomycin induced lung fibrosis model in male rats. Two cell concentrations (2 M cell/kg and 5 M cells/kg) and two types of vehicles for the cells were tested (PBS and CryoStor CS-10).

The development of fibrosis in rat lung after exposure to bleomycin is well documented in the literature and a frequently used model for studying the pathology of lung fibrosis and also the effect of different treatments. The number of LBX-THX-001 cells injected were chosen to be relevant for a possible human therapy. The number of cells were therefore chosen to reflect cell numbers used in previous studies on rats (8-20 M cells/kg) and humans (0.5-2 M cells/kg).

An intra-tracheal instillation of bleomycin (1000 U/rat) to 34 male SD-rats was used to induce lung fibrosis in the rats. During the first week, the rats were monitored and weighed daily and thereafter twice/week until termination of the study. At day 4 post bleomycin challenge, the LBX-THX-001 cells were administered by an intravenous (i.v.) injection. The injection volume was 194-535 μL (maximal tolerated injection volume 1 ml/kg). The response to the intra-tracheal instillation of bleomycin was as expected based on previous experience for the model with weight loss during the first days after instillation and thereafter recovery. There were no significant differences in weight loss between the bleomycin group and the treatment groups.

As shown in FIGS. 17A-D, bleomycin instillation induced fibrotic change in the lung. The histopathological evaluation concluded pathological changes in the bleomycin group both with regard to percent of parenchyma affected and after scoring using the modified Ashcroft scale. As shown in FIGS. 17A-D, the group treated with LBX-THX-001 cells (2 million cells/kg) 4 days post Bleomycin showed significantly less fibrosis in their lungs compared to the bleomycin group. This was seen both in the histopathological evaluation using the read-out “percent parenchyma affected” (FIGS. 17A-B) and the fibrosis scoring Ashcroft modified scale (FIGS. 17A-D). No human MSCs could be detected in rat lungs at termination (after 28 days).

Kidney TAF Cell Markers

Similar to the lung TAF MSC cell markers identified above, a number of surface markers of interest associated with kidney TAF cells were identified. For example, a non-exclusive list of surface markers used to identify and separate kidney TAF MSCs are provided below in Table 4. Similar to the lung TAF MSC markers, the surface markers identified in Table 4 may have at least a 12-fold increase in expression on prioritized kidney TAF clones compared to the average TAF-MSC clone (optionally with TPM threshold>2000). Moreover, as the number of different MSC-subtypes in TAF is limited, the selection of the tissue specific MSCs may be done first by characterization, and thereafter by a stepwise negative selection/sorting of the material by taking into account the combined (multivariate) surface marker profile of the different tissue specific MSC's. One of skill in the art will understand that any such combination of these surface markers may be used for identifying and isolation of kidney TAF cells from the general population of TAF-derived cells and/or TAF-MSC cells. In some examples, the below non-exclusive list of surface markers may be more highly expressed on the surface of kidney-TAF cells as compared to other cell types, such as other TAF-derived cells and/or TAF-MSC cells:

TABLE 4
Group C markers.

| | | |
|---|---|---|
| 1. | HAVCR1 | hepatitis A virus cellular receptor 1; |
| 2. | CD24 | CD24 molecule |
| 3. | CLDN6 | claudin 6; |
| 4. | ABCB1 | ATP binding cassette subfamily B member 1; |
| 5. | SHISA9 | shisa family member 9; |
| 6. | CRB3 | crumbs cell polarity complex component 3 |
| 7. | AC118754.1 | Arachidonate 15-lipoxygenase, ALOX15, Smoothelin- |
| | | like protein 2, SMTNL2, Glutathione hydrolase 6, |
| | | GGT6, Myb-binding protein 1A, MYBBP1A, Protein |
| | | spinster homolog 2, SPNS2 |
| 8. | ITGB6 | integrin subunit beta 6; |
| 9. | CDH1 | cadherin 1 |
| 10. | LSR | lipolysis stimulated lipoprotein receptor |
| 11. | EPCAM | epithelial cell adhesion molecule; |
| 12. | AJAP1 | adherens junctions associated protein 1; |
| 13. | ANO9 | anoctamin 9 |
| 14. | CLDN7 | claudin 7; |
| 15. | EFNA1 | ephrin A1; |
| 16. | MAL2 | mal, T cell differentiation protein 2 (gene/ |
| | | pseudogene) |
| 17. | F11R | F11 receptor |
| 18. | L1CAM | L1 cell adhesion molecule; |
| 19. | GFRA1 | GDNF family receptor alpha 1; |
| 20. | IGSF3 | immunoglobulin superfamily member 3; |
| 21. | TNF | tumor necrosis factor |
| 22. | MMP7 | matrix metallopeptidase 7; |
| 23 | FOLR1 | folate receptor alpha; |
| 24. | TGFA | transforming growth factor alpha |
| 25. | C3 | complement C3 |
| 26. | TNFSF10 | TNF superfamily member 10; |
| 27. | PDGFB | platelet derived growth factor subunit B; and/or |
| 28. | WWC1 | WW and C2 domain containing 1 |

As will be understood by one of skill in the art, suitable combinations of the markers listed in Table 4 may be used to separate kidney TAF cells from TAF-MSCs by selecting for specific markers from Table 4 or combinations of two, three, four, five, six or more markers from Table 4. In certain examples, kidney TAF MSCs can be more specifically identified by identifying a combination of stronger expression, such as 12-fold or more stronger expression (optionally with TPM threshold>2000) of any combination of the foregoing markers, e.g., HAVCR1 and/or CD24 and/or CLDN6 and/or ABCB1 and/or SHISA9 and/or CRB3 as compared to TAF-MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of kidney TAF MSCs (positive markers), in certain examples, the below surface markers may be more weakly expressed on kidney TAF cells as compared to other cell types (negative markers), such as such as ⅛-fold or less expression (optionally with TPM threshold>500) of any combination of the foregoing markers other TAF-derived cells and/or TAF-MSC cells: GREM1, PDGFRB, BGN, FAP, CXCL12, CCKAR, CD248. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate kidney TAF MSCs. In addition, those skilled in the art will also recognize that combinations including both negative and positive markers, such as at any of the thresholds described above, can also be effective to more specifically isolate kidney TAF MSCs.

Skin TAF Cell Markers

Similar to the lung and kidney TAF MSC markers identified above, a number of surface markers of interest associated with skin TAF cells were identified. For example, a non-exclusive list of surface markers used to identify and separate skin TAF cells are provided below in Table 5. The skin TAF MSC markers identified in Table 5 may have at least a 12-fold increase in expression on prioritized clones compared to the average TAF-MSC clone (optionally with TPM threshold>2000). Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of skin TAF cells from the general population of TAF-derived cells and/or TAF-MSC cells. In some examples, the below non-exclusive list of surface markers may be more highly expressed on the surface of skin-TAF cells as compared to other cell types, such as other TAF-derived cells and/or TAF-MSC cells:

TABLE 5
Group D markers.

| | | |
|---|---|---|
| 1. | TNFSF18 | TNF superfamily member 18; |
| 2. | PCDH19 | protocadherin 19; |
| 3. | NCAM2 | neural cell adhesion molecule 2; |
| 4. | TNFSF4 | TNF superfamily member 4; |
| 5. | CD248 | Endosialin; |
| 6. | DDR2 | discoidin domain receptor tyrosine kinase 2 |
| 7. | HTR2B | 5-hydroxytryptamine receptor 2B; |
| 8. | PCDH18 | protocadherin 18; |
| 9. | SULF1 | sulfatase 1; |
| 10. | MME | membrane metalloendopeptidase |
| 11. | ADGRA2 | adhesion G protein-coupled receptor A2; |
| 12. | DCSTAMP | dendrocyte expressed seven transmembrane protein; |
| 13. | PDGFRA | platelet derived growth factor receptor alpha; |
| 14. | UNC5B | unc-5 netrin receptor B; |
| 15. | SCUBE3 | signal peptide, CUB domain and EGF like domain |
| | | containing 3; |
| 16. | CEMIP | cell migration inducing hyaluronidase 1; |
| 17. | BDKRB1 | bradykinin receptor B1; |
| 18. | FLT1 | fms related tyrosine kinase 1 |
| 19. | BDKRB2 | bradykinin receptor B2; |
| 20. | FAP | fibroblast activation protein alpha |
| 21. | CASP1 | caspase 1; and/or |
| 22. | SRPX2 | sushi repeat containing protein X-linked 2 |

As will be understood by one of skill in the art, suitable combinations of the markers listed in Table 5 may be used to separate skin TAF MSCs from TAF-MSCs by selecting for specific markers from Table 5 or combinations of two, three, four, five, six or more markers from Table 5. In certain examples, skin TAF MSCs can be more specifically identified by identifying a combination of stronger expression, such as 12-fold or more stronger expression (optionally with TPM>2000) of any combination of the foregoing markers, e.g., TNFSF18 and/or PCDH19 and/or NCAM2 and/or TNFSF4 and/or CD248 and/or DDR2 as compared to TAF-MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of skin TAF cells (positive markers), in certain examples, the below surface markers may be more weakly expressed on skin TAF cells as compared to other cell types (negative markers), such as such as ⅛-fold or less expression (optionally with TPM threshold>500) of any combination of the foregoing markers other TAF-derived cells and/or TAF-MSC cells: CD24, TNFSF10, ITGB4, ABCB1. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate skin TAF MSCs. In addition, those skilled in the art will also recognize that combinations including both negative and positive markers, such as at any of the thresholds described above, can also be effective to more specifically isolate skin TAF MSCs.

Neural TAF Cell Markers

Similar to the lung, kidney, and skin TAF MSC markers identified above, a number of surface markers of interest associated with neural TAF cells were identified. For example, a non-exclusive list of surface markers used to identify and separate neural TAF cells are provided below. The neural TAF MSC surface markers identified in Table 6 may have at least a 3-fold increase in expression on prioritized clones compared to the average TAF-MSC clone (optionally with TPM threshold>500). Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of neural TAF cells from the general population of TAF-derived cells and/or TAF-MSC cells. In some examples, the below non-exclusive list of surface markers may be more highly expressed on the surface of neural-TAF cells as compared to other cell types, such as other TAF-derived cells and/or TAF-MSC cells:

TABLE 6
Group E markers.

| | | |
|---|---|---|
| 1. | HAVCR1 | hepatitis A virus cellular receptor 1; |
| 2. | ACKR3 | atypical chemokine receptor 3; |
| 3. | OSCAR | osteoclast associated Ig-like receptor; |
| 4. | C3 | complement C3 |
| 5. | SIRPB1 | signal regulatory protein beta 1; |
| 6. | SLC6A6 | solute carrier family 6 member 6; |
| 7. | CCKAR | cholecystokinin A receptor; |
| 8. | TNFSF10 | TNF superfamily member 10; |
| 9. | CLSTN2 | calsyntenin 2; |
| 10. | TENM2 | teneurin transmembrane protein 2; |
| 11. | SFRP1 | secreted frizzled related protein 1; |
| 12. | PIK3IP1 | phosphoinositide-3-kinase interacting protein 1; |
| 13. | SCNN1D | sodium channel epithelial 1 delta subunit; |
| 14. | CLDN11 | claudin 11; |
| 15. | ALDH3B1 | aldehyde dehydrogenase 3 family member B1; and/or |
| 16. | ITGB4 | integrin subunit beta 4 |

As will be understood by one of skill in the art, suitable combinations of the markers listed in Table 6 may be used to separate neural TAF MSCs from TAF-MSCs by selecting for specific markers from Table 6 or combinations of two, three, four, five, six or more markers from Table 6. In certain examples, neural TAF MSCs can be more specifically identified by identifying a combination of stronger expression, such as 3-fold or more stronger expression (optionally with TPM threshold>500) of any combination of the foregoing markers, e.g., HAVCR1 and/or ACKR3 and/or OSCAR and/or C3 and/or SIRPB1 and/or SLC6A6 as compared to TAF-MSCs. When using combinations of markers, identification may be achieved with a lower threshold of stronger expression, such as 2-fold or more or a higher threshold such as 6-fold or more, 8-fold or more, or 12-fold or more expression of each of the markers. In addition, those skilled in the art will also recognize that combinations including both negative and positive markers, such as at any of the thresholds described above, can also be effective to more specifically isolate neural TAF MSCs.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing examples. The disclosure extends to any novel one, or any novel combination, of the features disclosed in this specification (including any accompanying claims, abstract and drawings), or to any novel one, or any novel combination, of the steps of any method or process so disclosed.

Those skilled in the art will appreciate that in some examples, the actual steps taken in the processes illustrated or disclosed may differ from those shown in the figures. Depending on the example, certain of the steps described above may be removed, others may be added. For example, the actual steps or order of steps taken in the disclosed processes may differ from those shown in the figure. Depending on the example, certain of the steps described above may be removed, others may be added. Furthermore, the features and attributes of the specific examples disclosed above may be combined in different ways to form additional examples, all of which fall within the scope of the present disclosure.

Conditional language, such as “can”, “could”, “might”, or “may”, unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular example. The terms “comprising”, “including”, “having”, and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each”, as used herein, in addition to having its ordinary meaning, can mean any subset of a set of elements to which the term “each” is applied. Additionally, the words “herein”, “above”, “below”, and words of similar import, when used in this application, refer to this application as a whole and not to any particular portions of this application.

Conjunctive language such as the phrase “at least one of X, Y, and Z”, unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately”, “about”, “generally”, and “substantially” as used herein represent a value, amount, or characteristic close to the stated value, amount, or characteristic that still performs a desired function or achieves a desired result. For example, the terms “approximately”, “about”, “generally”, and “substantially” may refer to an amount that is within less than 10% of, within less than 5% of, within less than 1% of, within less than 0.1% of, and within less than 0.01% of the stated amount. As another example, in certain examples, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain examples of the disclosure are encompassed in the claim set listed below or presented in the future.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. Embodiments and features of the present invention are also outlined in the following items and also illustrated by the following non-limiting examples.

### EXAMPLES

### Example 1—Porcine Model for Acute Respiratory Distress Syndrome (ARDS)

### Materials and Methods

### Ethical Considerations

The local Ethical Committee for Animal Research (Dnr 8401/2017) reviewed and approved all procedures in this study. All animals received care according to the USA Principles of Laboratory Animal Care of the National Society for Medical Research, Guide for the Care and Use of Laboratory Animals, National Academies Press (1996).

### Animal Preparation

Before each experiment, the blood type of 24 adult Yorkshire pigs was determined using Seraclone™ Anti-A (blood grouping reagent, Bio-Rad, Medical Diagnostics GmbH, Dreieich, Germany). Randomisation of the pigs was performed prior to the beginning of the study and animals were assigned to the treatment or non-treatment group, accordingly. All donor animals were administered with LPS (Sigma-Aldrich, O111: B4, Sigma-Aldrich, Merck KGaA, Darmstadt, Germany) to induce ARDS. ARDS lungs of the non-treated group received EVLP and LTx without further experimental treatment. The lungs of the treated group and recipient thereof were treated with a total of three doses cell injections.

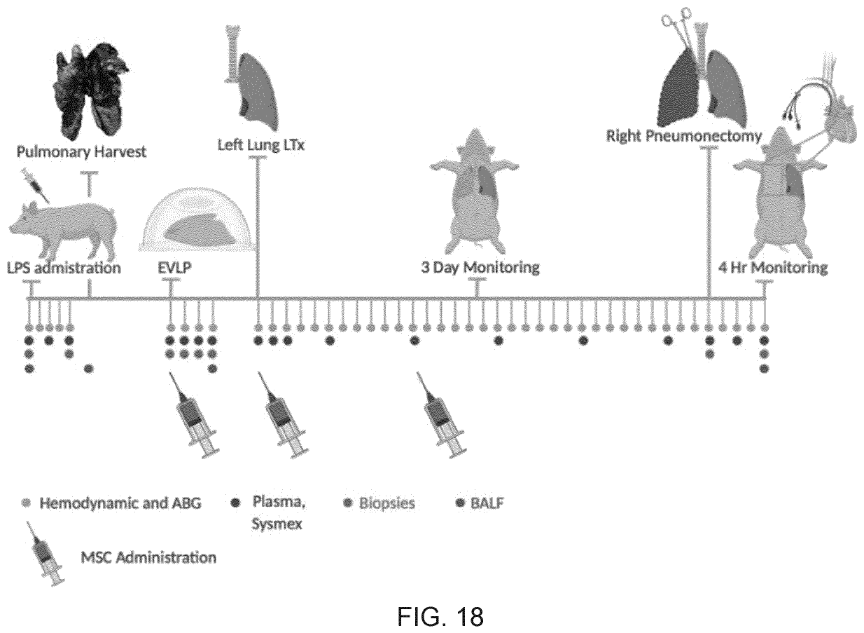

All pigs were premedicated with ketamine (Ketaminol® vet. 100 mg/ml; Farmaceutici Gellini S.p.A., Aprilia, Italy; 20 mg/kg) and xylazine (Rompun® vet. 20 mg/ml; Bayer AG, Leverkusen, Germany; 2 mg/kg). General anaesthesia was established and maintained through infusions with ketamine (Ketaminol® vet, Intervet AB, Stockholm, Sweden), midazolam (Midazolam Panpharma®, Oslo, Norway) and fentanyl (Leptanal®, Lilly, France). A urinary catheter was inserted in the bladder, and a peripheral intravenous (IV) line placed in the earlobe. A 7.5 size endotracheal tube was utilized for intubation. Mechanical ventilation was performed with a Siemens-Elema ventilator (Servo 900C, Siemens, Solna, Sweden), to maintain carbon dioxide levels (PaCO₂) between 33-41 mmHg and the tidal volume (Vt) was kept at 6-8 ml/kg. An arterial line (Secalon-T™, Merit Medical Ireland Ltd, Galway, Ireland) was inserted in the right common carotid artery and a pulmonary artery catheter (Swan-Ganz CCOmbo V and Introflex, Edwards Lifesciences Services GmbH, Unterschleissheim, Germany) placed in the right internal jugular vein. 12 pigs served as donors and 12 pigs as recipients. An overview of the experimental setup is illustrated in FIG. 18. Prior to initiation of surgery, dihydrostreptomycinsulfate (0.1 ml/kg) (Boehringer Ingelheim Animal Health Nordics A/S, Copenhagen, Denmark) was given subcutaneously.

ARDS Induction with LPS in Donors

Lipopolysaccharide (LPS) from Gram-negative Escherichia coli bacteria (O111:B4, Sigma-Aldrich, Merck KGAA, Darmstadt, Germany) was administered to induce an acute respiratory distress syndrome (ARDS) in donors according to the Berlin definition (Force et al., 2012). This procedure has been previously described (Stenlo et al., 2020). The saline solution (Baxter Viaflo 9 mg/ml, Baxter International, Deerfield, IL, USA) was used for LPS dilution to 2 mg/ml. The LPS solution was administered intravenously as an infusion (2 μg/kg/min) for one hour and reduced by 50% for another hour afterwards. Following LPS administration, all animals developed hemodynamic instability, requiring continuous infusion of norepinephrine (40 μg/ml, 0.05-2 μg/kg/min) (Pfizer AB, Sollentuna, Sweden) and dobutamine (2 mg/ml, 2.5-5 μg/kg/min) (Hameln Pharma Plus GmbH, Hameln, Germany). Ringer's acetate (Baxter Medical AB, Kista, Sweden) was generally utilised to compensate fluid loss. ARDS stages were defined according to the Berlin definition of ARDS (Force et al., 2012) based on the PaO₂/FiO₂ ratio. ARDS was confirmed if two separate arterial blood gas measurements within a 15-minute interval met the PaO₂/FiO₂ range defined in the Berlin guidelines. A ratio between 201-300 mmHg was defined as mild, between 101-200 mmHg as moderate ARDS, and ≤ 100 mmHg as severe ARDS.

### Arterial Blood Gas (ABG) and Hemodynamic Measurements

Arterial blood gases were analysed with an ABL 90 FLEX blood gas analyser (Radiometer Medical ApS, Brønshøj, Denmark). According to clinical samples, blood was analysed every 30 minutes in the donors, every hour during EVLP and following transplantation in the recipients.

Hemodynamic parameters were measured and recorded every 30 minutes in the donor as well as recipients after transplantation using thermodilution with an arterial line and Swan-Ganz catheter. Parameters analysed were heart rate (HR), systolic blood pressure (SBP), diastolic blood pressure (DBP), mean arterial pressure (MAP), central venous pressure (CVP), cardiac output (CO), systolic pulmonary pressure (SPP), diastolic pulmonary pressure (DPP), mean pulmonary pressure (MPP), pulmonary artery wedge pressure (PAWP), systemic vascular resistance (SVR), and pulmonary vascular resistance (PVR).

Pulmonary Harvest after Confirmed ARDS—Donor

After confirmation of ARDS, a median sternotomy was performed. The pulmonary artery was cannulated via the right ventricle with a 28 F cannula secured by a purse string suture placed in the outflow tract of the pulmonary artery. A clamp was put on the superior vena cava, the inferior vena cava, and on the ascending aorta. The left atrium and inferior vena cava were opened. The lungs were perfused antegradely with 4 L of cold Perfadex® PLUS solution (XVIVO perfusion, Gothenburg, Sweden) distributed at a low perfusion pressure (<20 mmHg). The lungs were harvested en bloc in a standard fashion, immersed in cold Perfadex® PLUS solution, and put in cold storage at 4° C. for 2 hours.

### Results

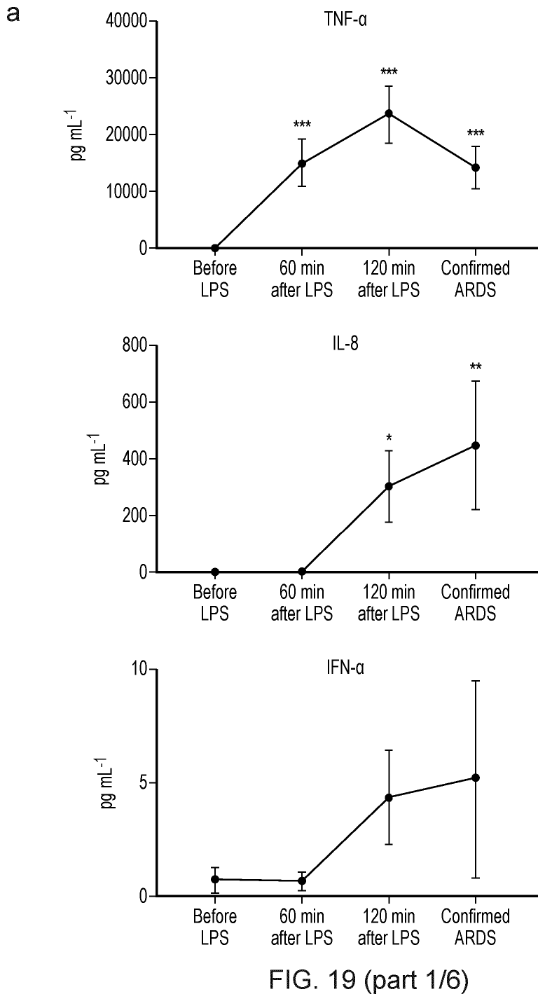

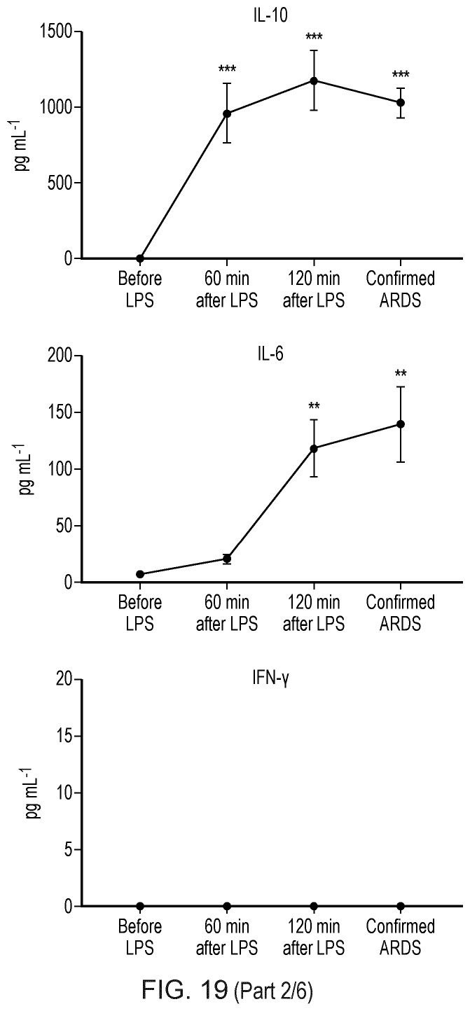

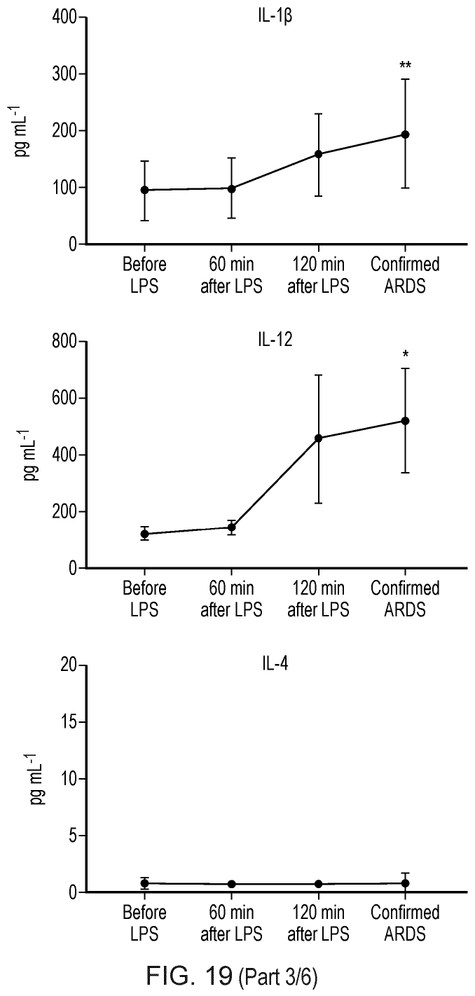

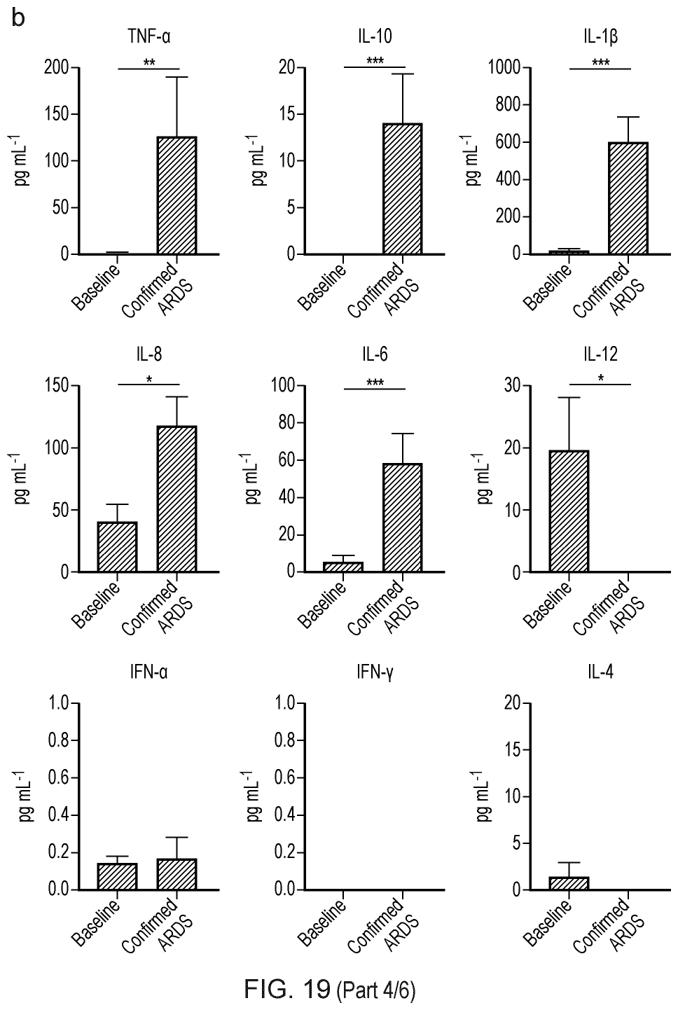

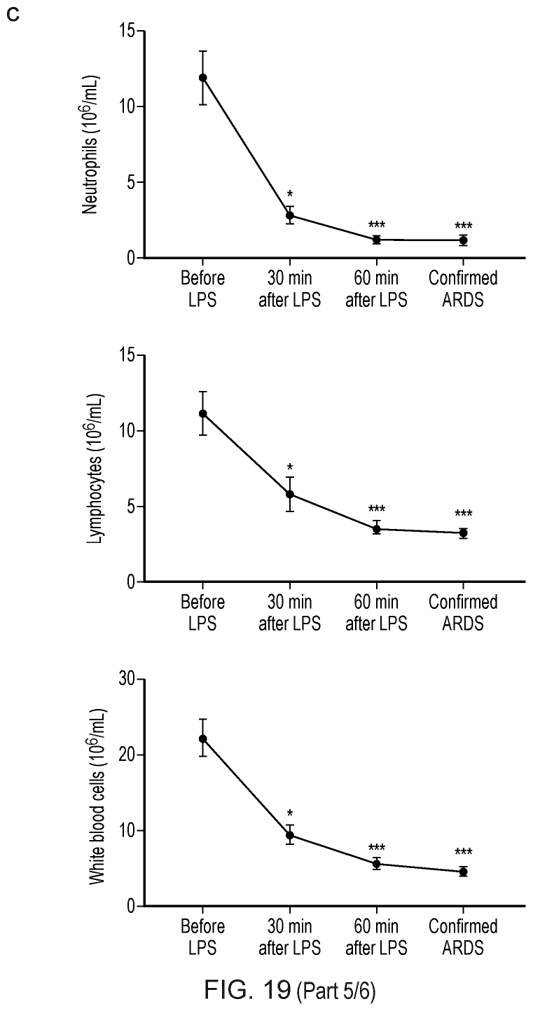

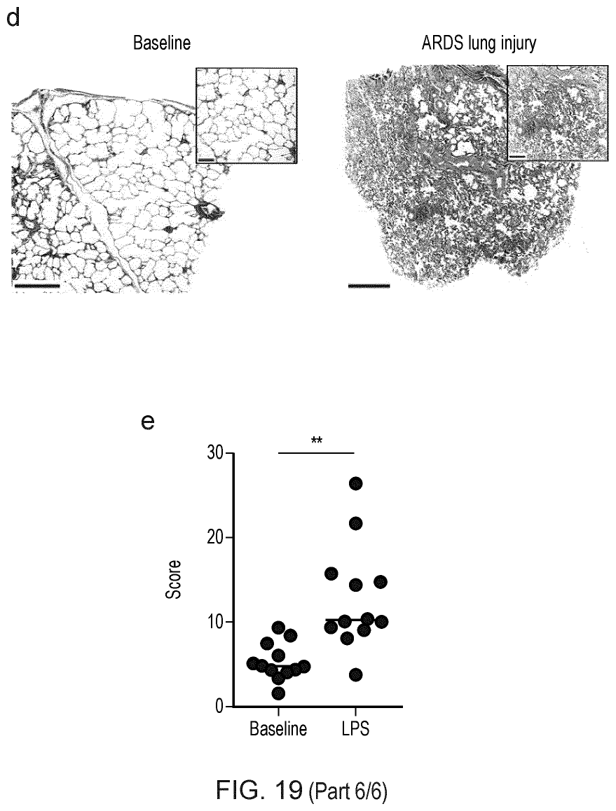

The LPS model in pigs for studying ARDS is known. This model was replicated for these data and confirmed based on a cytokine panel; cell count of neutrophils, lymphocytes and total white blood cells; and histology of lung sections. All pigs treated with LPS developed ARDS (FIG. 19a-e), as characterised by the expected cytokine storm following LPS treatment and the evident reduction in the alveolar space, demonstrating lung injury in the histology section.

Accordingly, this is a reproducible model for establishing ARDS that can be used to assess the impact of treatment with MSCs.

### Discussion

There are a number of advantages to using pigs to study EVLP. For example, the experimental parameters can be directly applied to human subjects; the size and weight of pigs are similar to humans; and proper tidal volume, positive end-expiratory pressure (PEEP), and perfusion time settings can be used as a basis for clinical trials (Pan et al., 2018). The establishment and use of this pig model are therefore relevant for extrapolating its data to the human setting.

### Example 2—Ex Vivo Lung Perfusion (EVLP)

### Materials and Methods

The pigs used in these experiments were prepared as outlined in Example 1.

### Ex Vivo Lung Perfusion (EVLP)

EVLP was performed using Vivoline LS1 (XVIVO perfusion, Gothenburg, Sweden) combined with the Toronto protocol. The target perfusion was 40% of cardiac output, with a tidal volume of 7 ml/kg body weight of the donor, respiratory rate (RR) of 7.5 cm H₂O PEEP, and 21% FiO₂ for 4 hours (Van Raemdonck et al., 2015 and Yeung et al., 2011). Steen™ Solution (XVIVO perfusion, Gothenburg, Sweden) with blood drawn from the respective donor animal prior to LPS treatment was used to prime the system to reach a hematocrit level of 15-20% in the circuit. If the perfusate level dropped below 300 ml in the reservoir, additional Steen solution (XVIVO Perfusion) was added. EVLP physiology was recorded hourly during the 4-hour perfusion period. After 4 hours in EVLP, the lungs were cooled down to 8-12° C. for approximately 45 minutes before transplantation.

### Stem Cell Administration During EVLP

Human mesenchymal stem cells (MSCs) isolated from full term amniotic fluid (TAF) were obtained from voluntary healthy donors. The MSCs were selected based on CD248 (i.e. for lung TAF MSCs) and expanded in culture under GMP conditions to meet requirements for clinical doses, followed by cryopreservation for off the shelf use. For each MSC infusion, 2×10⁶ cells/kg recipient were thawed in a 37° C. water bath and washed with phosphate buffered saline solution (PBS, HyClone, GE Healthcare Life Sciences, Chicago, IL, USA) and suspended in 50 ml PBS. The MSCs were administered intravenously over the course of 10 minutes at the start of EVLP in the treated group. The non-treated group received 50 ml of PBS as placebo treatment.

### Blood Cell Counts

Lymphocytes, neutrophils, and total white blood cell counts were measured in whole blood anti-coagulated with EDTA using a Sysmex KX-21N automated hematology analyzer (Sysmex, Milton Keynes, UK). Blood was analysed every 30 minutes in the donors and every hour during EVLP. Blood samples were analysed as soon as possible, within a maximum of 8 hours, and kept at room temperature until analysis.

### Measurements of Cytokine and Chemokine Levels in Plasma and Bronchoalveolar Lavage Fluid (BALF)

Measurements of cytokine and chemokine levels in the plasma were taken at baseline, every 60 minutes in the donor animals, and every hour during EVLP. These levels were analysed with the multiplexed Cytokine & Chemokine 9-Plex Porcine ProcartaPlex™ Panel 1 kit (Thermo Fisher Scientific Cat. No. EPX090-60829-901) according to the manufacturer's instructions. Sample analysis was performed using a Bioplex-200 system (BioRad, Hercules, CA, USA). The nine cytokines IL-1β, IL-4, IL-6, IL-8, IL-10, IL-12p40, IFN-α, IFN-γ and TNF-α were evaluated. BALF was collected at baseline and before lung harvest in donors, and at the end of EVLP in the donor lung.

### Histology and Tissue Wet-Dry Weight Ratio

Baseline lung biopsies were taken through a right thoracotomy from the right lobe before the start of LPS administration. Furthermore, biopsies were collected from the right lower lobe right before lung harvest after ARDS was confirmed. After connecting the lung to the EVLP, biopsies were taken from the right lower lobe at initiation, followed by further collection of biopsies every hour throughout EVLP. The tissue was fixed in 10% neutral buffered formalin solution (Sigma Aldrich, Germany) at 4° C. overnight. Formalin-fixed biopsies were subjected to a graded ethanol series and isopropanol (both Fisher Scientific, UK) prior to paraffin embedding (Histolab, Västra Frölunda, Sweden). After de-paraffinization, the tissue was cut into 4 μm sections. The sections were stained with hematoxylin and eosin (Merck Millipore, Germany) followed by consecutive dehydration in graded ethanol and xylene solutions. The dried sections were mounted with Pertex (Histolab) and bright-field images were acquired with using a Nikon Eclipse Ts2R microscope (Nikon, Tokyo, Japan).

Lung injury scoring was performed for each pig independently by three blinded scorers with experience in porcine lung injury models. Scoring criteria were number of inflammatory cells, presence of hyaline membranes, level of proteinaceous debris, thickening of the alveolar wall, enhanced injury, hemorrhage and atelectasis using a modification of previously described scoring methodology (Matute-Bello et al., 2011). The scores were given on a scale of 0 to 8 for each feature. The average of the sum of the characteristic scores was used to determine the overall lung injury score.

Furthermore, pulmonary oedema was determined by measuring the wet weight to dry weight ratio of lung tissue from the lower lobe after EVLP in the left lung. Proximal lung tissue pieces were weighed, lyophilized for 24 h, and re-weighed. The ratio between the wet and dry weight was then calculated.

### Primary Graft Dysfunction (PGD) Staging

Primary graft dysfunction (PGD) was staged based on the PaO₂/FiO₂ ratio according to the ISHLT guidelines (Snell et al., 2017).

### Calculations and Statistics

Continuous variables were reported as mean±standard error of the mean (SEM). Statistically significant differences between groups were tested with the Student's T-test and within groups using analysis of variance (ANOVA) if data were normally distributed. If data were not normally distributed, the Mann-Whitney test and the Wilcoxon test were applied instead. A Chi-Squared test was performed to analyse observed frequencies of categorical variables. All statistical analysis was performed using GraphPad Prism software (Version 8, GraphPad, San Diego, USA). Statistical significance was generally defined as: p<0.001 (***), p<0.01 (**), p<0.05 (*), and p>0.05 (not significant, ns).

### Results

IL-1β is a known biomarker for distinguishing between what would be considered a ‘good lung’ from a ‘bad lung’. IL-1β is an inducible proinflammatory cytokine that is not generally expressed in healthy cells or tissue. The release of IL-1β can cause pulmonary inflammation and fibrosis. Accordingly, a lower level of IL-1β is a favourable outcome within the context of lung physiology and repair.

Treatment with MSCs resulted in a significant reduction in IL-1β (see FIG. 20), with the level of IL-1β remaining below 100 μg/ml in plasma for the entire duration of EVLP. On the other hand, in the PBS control group, IL-1β is already nearly 3 time higher at 1 hour of EVLP and continues to rise as EVLP continues.

The reduction in IL-1β by the MSC treatment will result in a lower risk of pulmonary inflammation and fibrosis.

IFN-α is a known biomarker for activating macrophages. Macrophages have an important role in lung repair and the resolution of inflammation. Accordingly, a higher level of IFN-α is a favourable outcome within the context of lung physiology and repair.

Treatment with MSCs resulted in a significant increase in IFN-α (see FIG. 21), with the level of IFN-α reaching 10 μg/ml in plasma and being sustained for the duration of EVLP. On the other hand, in the PBS control group, IFN-α remains at <1 μg/ml for the duration of EVLP.

The increase in IFN-α by the MSC treatment provides an environment that supports macrophage activation for resolution of inflammation and lung tissue repair. Therefore, lungs conditioned by the MSC treatment are in an improved condition for use following EVLP.

Arterial blood gas oxygen tension/fraction of inspire oxygen ratio (PaO₂/FiO₂) is a useful biomarker for predicting subsequent outcomes of early graft dysfunction, as it can demonstrate the capability of lungs to oxygenate blood.

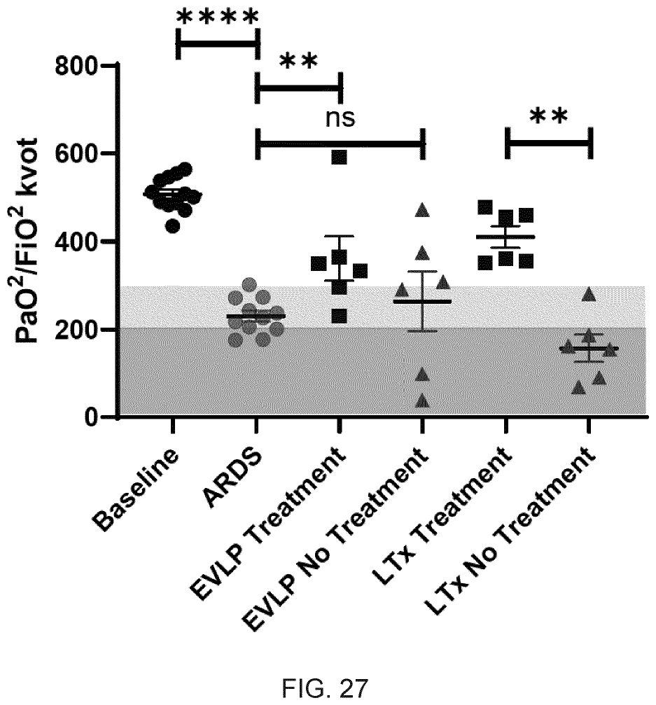

In FIG. 27, the pigs that received the MSC treatment during EVLP had significantly improved PaO₂/FiO₂ ratio compared with the ARDS group, with all treated lungs indicating a PaO₂/FiO₂ ratio that would not be classified as severe PGD. On the other hand, the PBS control group were not significantly different compared with ARDS, with two of the six pigs having a PaO₂/FiO₂ ratio that would indicate severe PGD. These data demonstrate that the MSC treatment reliably improves lung functionality in this ARDS porcine model.

Overall, the use of MSCs reduced sign of acute lung injury/ARDS after treatment in EVLP, and MSCs significantly reduced PGD.

### Discussion

These data demonstrate for the first time that TAF MSCs can be used in EVLP and exert their therapeutic effects for EVLP when delivered intravenously. Previously, it has been demonstrated that intrabronchial administered of MSCs derived from human umbilical cord fails to achieve MSC retention in the lung parenchyma (Mordant et al., 2016), and that delivery via the pulmonary artery was more optimal. Mordant et al. performed a dose-escalation study administering 50×10⁶, 150×10⁶ or 300×10⁶ MSCs via the pulmonary artery, identifying an optimal tolerated dose to be 150×10⁶ MSCs for a 30 kg pig, i.e. an optimal dose of 5×10⁶ MSCs/kg. Interestingly, the optimal dose of human umbilical cord derived MSCs used by Mordant et al. failed to achieve a statistically significant difference in the level of IL-1β and IL-10 between MSC-treated and the control group during the 12 hours of EVLP conducted in the study. On the other hand, despite administering less than half the number of TAF MSCs compared with human umbilical cord MSCs, a significant difference is observed for IL-1β throughout the duration of EVLP.

These data are further surprising given the administration route of intravenous delivery (“IV” delivery; note that although “IV” may also indicate “intravascular” in the art, which would include arterial delivery, “IV” herein is to be interpreted as “intravenous”). IV delivery has been attempted in rodent (rat and mouse) models, wherein human MSCs delivered to such models were rapidly retained in the microvasculature due to their size (Pacienza et al., 2019). This is confirmed in Mordant et al., who emphasised that intravenous injection of MSCs resulted in about 90% of cells being trapped in the lung vasculature, thereby failing to reach the lung parenchyma where they exert their effects. However, unlike the types of MSCs intravenously injected previously in the art, TAF MSCs capably exert their effects on the lungs despite intravenous delivery.

Accordingly, not only are the TAF MSCs exerting a more significant anti-inflammatory effect on the lungs through their use in EVLP than human umbilical cord MSCs, but they can also be delivered via routes that fail for other such types of MSCs.

### Example 3—Lung Transplantation (LTx)

### Materials and Methods

The pigs used in these experiments were prepared as outlined in Examples 1 and 2.

### Stem Cell Administration During Lung Transplantation

TAF MSCs were prepared as described in Example 2.

For each MSC infusion, 2×10⁶ cells/kg recipient were thawed in a 37° C. water bath and washed with phosphate buffered saline solution (PBS, HyClone, GE Healthcare Life Sciences, Chicago, IL, USA) and suspended in 50 ml PBS. The MSCs were administered intravenously over the course of 10 minutes at 1 h and 12 h after transplantation in the treated group. The non-treated group received 50 ml of PBS as placebo treatment, respectively.

### Left Lung Transplantation and Recipient Follow Up

Lung transplantation was performed as described by Mariscal et al., 2018. In brief, the pulmonary hilum was dissected through a left thoracotomy. The left pulmonary artery, left bronchus, and left atrium were clamped individually, followed by a left pneumonectomy of the native left lung. The donor lung was sewn in and the anastomosis of the bronchus was sutured using polydioxanone sutures (PDS 4-0, Ethicon, Somerville, NJ, USA). The atrial cuff and the pulmonary artery were sutured with polypropylene (Prolene 5-0, Ethicon, Somerville, NJ, USA) with a continuous pattern. After suturing the bronchus, a bronchoscopy was done to confirm an open bronchial anastomosis. All recipients were continuously immunosuppressed using tacrolimus (0.15 mg/kg, orally) (Sandoz AS, Copenhagen, Denmark) and methylprednisolone sodium succinate (1 mg/kg, intravenously, twice daily) (Solumedrol, Pfizer, New York, USA).

The recipient animals were kept under anaesthesia with infusions of ketamine (Ketaminol® vet), midazolam (Midazolam Panpharma®, Oslo, Norway) and fentanyl (Leptanal®, Lilly, France). The pigs received 500 mg imipenem (Merck & Co. Inc., Kenilworth, NJ, USA) intravenously 3 times per day throughout the experiments. All animals were followed up for at least 48 hours, some were followed up for 60-72 hours. Once a day, dihydrostreptomycin sulfate (0.1 ml/kg) (Boehringer Ingelheim Animal Health Nordics A/S, Copenhagen, Denmark) was given subcutaneously.

### Right Pneumonectomy

The pulmonary hilum was dissected through a mid-sternotomy. To assess the isolated function of the transplanted left lung, a right pneumonectomy was performed, and the recipient was followed for additional 4 hours before terminating the experiment. During these 4 hours, the recipient was additionally monitored using a Swan-Ganz catheter as described above in animal preparation.

### Blood Cell Counts

Lymphocytes, neutrophils, and total white blood cell counts were measured in whole blood anti-coagulated with EDTA using a Sysmex KX-21N automated hematology analyzer (Sysmex, Milton Keynes, UK). Blood was analysed every 30 minutes in the donors and every 1-6 hours after LTx in the recipients. Blood samples were analysed as soon as possible, within a maximum of 8 hours, and kept at room temperature until analysis.

### Measurements of Cytokine and Chemokine Levels in Plasma and Bronchoalveolar Lavage Fluid (BALF)

Measurements of cytokine and chemokine levels in the plasma were taken at baseline, every 60 minutes in the donor animals, every hour for the first 3 hours, and then every 6 hours in the recipient animal. These levels were analysed with the multiplexed Cytokine & Chemokine 9-Plex Porcine ProcartaPlex™ Panel 1 kit (Thermo Fisher Scientific Cat. No. EPX090-60829-901) according to the manufacturer's instructions. Sample analysis was performed using a Bioplex-200 system (BioRad, Hercules, CA, USA). The nine cytokines IL-1β, IL-4, IL-6, IL-8, IL-10, IL-12p40, IFN-α, IFN-γ and TNF-α were evaluated. BALF was collected at baseline and before lung harvest in donors, and at the end of the experiment in the recipients.

### Histology and Tissue Wet-Dry Weight Ratio

Baseline lung biopsies were taken through a right thoracotomy from the right lobe before the start of LPS administration. Furthermore, biopsies were collected from the right lower lobe right before lung harvest after ARDS was confirmed. At the termination of the experiment, biopsies were collected from the transplanted left lung. The tissue was fixed in 10% neutral buffered formalin solution (Sigma Aldrich, Germany) at 4° C. overnight. Formalin-fixed biopsies were subjected to a graded ethanol series and isopropanol (both Fisher Scientific, UK) prior to paraffin embedding (Histolab, Västra Frölunda, Sweden). After de-paraffinization, the tissue was cut into 4 μm sections. The sections were stained with hematoxylin and eosin (Merck Millipore, Germany) followed by consecutive dehydration in graded ethanol and xylene solutions. The dried sections were mounted with Pertex (Histolab) and bright-field images were acquired with using a Nikon Eclipse Ts2R microscope (Nikon, Tokyo, Japan).

Lung injury scoring was performed for each pig independently by three blinded scorers with experience in porcine lung injury models. Scoring criteria were number of inflammatory cells, presence of hyaline membranes, level of proteinaceous debris, thickening of the alveolar wall, enhanced injury, hemorrhage and atelectasis using a modification of previously described scoring methodology (Matute-Bello et al., 2011). The scores were given on a scale of 0 to 8 for each feature. The average of the sum of the characteristic scores was used to determine the overall lung injury score.

Furthermore, pulmonary oedema was determined by measuring the wet weight to dry weight ratio of lung tissue from the lower lobe after 48 hours of transplantation in the left (transplanted) lung. Proximal lung tissue pieces were weighed, lyophilized for 24 h, and re-weighed. The ratio between the wet and dry weight was then calculated.

### Primary Graft Dysfunction (PGD) Staging

Primary graft dysfunction (PGD) was staged based on the PaO₂/FiO₂ ratio according to the ISHLT guidelines (Snell et al., 2017).

### Calculations and Statistics

Continuous variables were reported as mean±standard error of the mean (SEM). Statistically significant differences between groups were tested with the Student's T-test and within groups using analysis of variance (ANOVA) if data were normally distributed. If data were not normally distributed, the Mann-Whitney test and the Wilcoxon test were applied instead. A Chi-Squared test was performed to analyse observed frequencies of categorical variables. All statistical analysis was performed using GraphPad Prism software (Version 8, GraphPad, San Diego, USA). Statistical significance was generally defined as: p<0.001 (***), p<0.01 (**), p<0.05 (*), and p>0.05 (not significant, ns).

### Results

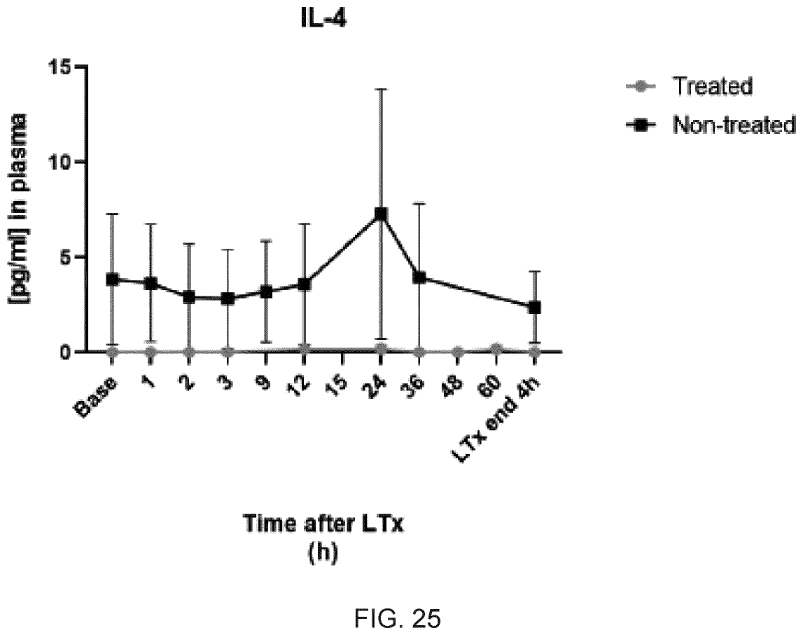

Following lung transplantation, the concentration of a number of cytokines in plasma was assessed. FIGS. 22, 23, 24 and 25 show the concentrations for IL-8, TNF-α, IL-1β and IL-4, respectively. IL-8 is a proinflammatory cytokine that is part of the same family as IL-1B. TNF-α and IL-4 are further proinflammatory cytokines.

For each of the proinflammatory cytokines assessed after lung transplantation, treatment with MSCs resulted in a significant reduction in plasma concentration that is sustained up to 60 hours in pigs and remains low following the right lung pneumonectomy at the end of the 4-hour monitoring stage (see “LTx end 4 h” in each graph). These data demonstrate that the administration of MSCs following transplantation maintains an anti-inflammatory environment in the lung beyond 60 hours, even when the cells are administered at a much earlier time point, and even following removal of the native right lung.

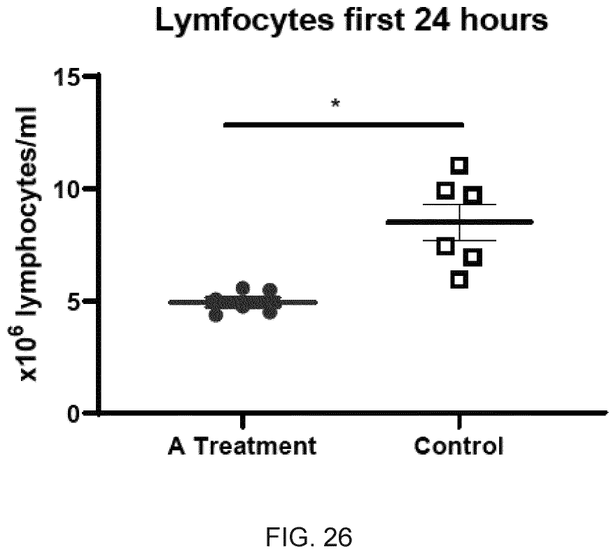

Lymphocytes are key for driving the pathology that follows PGD, and so the number of lymphocytes in the first 24 hours following transplantation was also assessed (see FIG. 26). The MSC treated group (“A Treatment”) had significantly fewer lymphocytes per ml of blood, indicating a reduced level of lymphocyte infiltration into the transplant.

In FIG. 27, the pigs that received the MSC treatment following transplantation had significantly improved PaO₂/FiO₂ ratio compared with the control “No Treatment” group, with all treated lungs being free of PGD, irrespective of PGD severity. On the other hand, 5/6 pigs in the PBS control group developed severe PGD, with the remaining pig being assessed as having moderate PGD. These data demonstrate that the MSC treatment during transplantation prevents the development of PGD.

Overall, the use of MSCs improved pulmonary graft function after transplantation, significantly reduced PGD, and significantly reduced the needed amount of inotropic support after transplantation. Furthermore, in all cases, no adverse events were noted.

### Discussion

These data demonstrate for the first time that TAF MSCs can be used in lung transplantation and exert their therapeutic effects for transplantation when delivered intravenously. As discussed in Example 2, previous studies have focused on the use of human umbilical cord derived MSCs in EVLP. In Nakajima et al., 2019, a similar lung transplantation model is performed following the use of the preferred concentration of 5×10⁶ human umbilical cord derived MSCs as described in Mordant et al., 2016. One of the drawbacks of the experimental protocol used by Nakajima et al. is the absence of any readouts at suitable times following occlusion of the right native pulmonary artery and main-stem bronchus. While the right lung remains in place and functional (i.e. not occluded), it will compensate for any potential deficiencies caused by the left lung transplant. Nakajima et al. mention that occlusion of the right native pulmonary artery and main-stem bronchus is performed at 4 hours after transplantation to independently assess the transplanted lung function. However, the group measures paracrine soluble factors and wet-to-dry ratio (to indicate pulmonary oedema) at 4 hours after transplant. Therefore, the data of Nakajima et al. is limited to a setting in which the right lung remains intact and healthy, potentially obscuring each of the readouts.

In the present transplantation study, a 4-hour monitoring window, with samples obtained at each hour, is included after right pneumonectomy. The right pneumonectomy replicates that of the occlusion suggested (but not adequately assessed) in Nakajima et al. by removing the confounding variable of a healthy lung compensating for the left lung transplant. Therefore, these data demonstrate for the first time that use of TAF MSCs in lung transplantation reduces plasma levels of various inflammatory cytokines (IL-8, TNF-α, IL-1β and IL-4), and sustains the reduced levels in setting where the only functional lung is the transplanted lung (i.e. following right pneumonectomy).

These data have important implications for the human setting, and particularly bilateral transplantation (i.e. where no ‘healthy’ lung remains that could compensate for an underperforming transplanted lung). Where the data of Nakajima et al. may be limited to single lung transplants reliant on the presence of a healthy lung, the present data advance on this to show that the lack of a healthy second lung retains the therapeutic benefit of the MSCs. Accordingly, the use of TAF MSCs is likely to be applicable not only to single lung transplant, but also bilateral lung transplant.

Another advantage of the present study over that of Nakajima et al. is the overall duration. Nakajima et al. acknowledged that a limitation of their study is that the observed time of 4 hours after transplantation was short. However, in the present application, the pigs were followed up for at least 48 hours following transplantation, with samples obtained at multiple time points throughout. These data culminate in a more robust assessment of the transplantation of lungs treated with MSCs. Although the right lung remains intact during the initial follow-up, the data are not significantly different for the various follow-up time points and following right pneumonectomy. Therefore, it is evident in the present application that the reduction in cytokine levels in plasma is attributable to the TAF MSCs and not a compensatory mechanism of the right lung remaining present.

### Example 4—Effect on TAF MSC's on T-Cell Activation and Macrophage Activation/Polarization Using Human Peripheral Blood Mononuclear Cells (PBMCS)

### 1.1 Aim

The aim of the study was to evaluate the effect of unsorted human Mesenchymal Stem Cells (MSCs) on T cell activation and macrophage activation/polarization using human Peripheral Blood Mononuclear Cells (PBMCs).

### 1.2 Ex Vivo Analysis

Freshly isolated human PBMCs, pooled from three donors, were activated with anti-CD3/anti-CD28 at 10 μg/ml and 5 μg/ml respectively in presence of MSCs ex vivo for 24 or 72 hours. Following activation, cells were analysed for effects on cellular composition after 24 hours and for effects on cellular composition and cell proliferation using CFSE after 72 hours. In addition, supernatants collected after 24 hours of activation were analysed for cytokine levels using Luminex.

### 2. Materials and Methods

### 2.1 Reagents

AbC Total Compensation capture beads (Life Technologies, A10497)
AD-MSCs (provided by Sponsor)
Anti-CD3 (Nordic Biosite, 300438)
Anti-CD28 (Nordic Biosite, 302934)
Anti-human CD4—PerCp (Nordic Biosite, 344624)
Anti-human CD8—QDot800 (Thermo Fisher, Q22157)
Anti-human CD80—BV421 (Nordic Biosite, 305222)
Anti-human CD73—FITC (Nordic Biosite, AM26144FC-N)
Anti-human PD-1—PE (Nordic Biosite, 329906)
Anti-human CD163—BV605 (Nordic Biosite, 333616)
Anti-human CD206—BV711 (Nordic Biosite, 321136)
Cell culture medium (StemMACS MSC expansion Media, Miltenyi (M3))
Cell culture medium (Prime-XV MSC Expansion XSFM, IrvineScientific (M4))
Carboxyfluorescein succinimidyl ester (CFSE), (Sigma, 21888)
HBSS (Gibco, Life Technologies, 14175)
Human MSCs (provided by Sponsor)
Human PBMCs (Blodcdentralen, Lunds Universitetssjukhus, Lund)
Luminex 12-plex (RnD Systems, LXSAHM-12)
mqH20 (QPAK1, Millipore)
Negative beads (Life Technologies, A10497)
PBS (Gibco, Life Technologies, 14190)

### 2.2 Equipment

Attune Nxt (ThermoFisher Scientific, Sunnyvale, California, USA)
Scepter cell counter (Millipore Merck, MA, USA) SpectraMax
Luminex 200 (Bio-Rad, Solna, Seden)
Thermo scientific cell culture plate 96-well U bottom (Thermo Fisher, 168136)
V-bottom plates (Nunc, 732-0191)
Falcon Tubes (VWR, 734-0443)

### 2.3 Procedures

### 2.3.1 Ex Vivo Assay

Peripheral Blood Mononuclear Cells (PBMCs) were isolated from leucocyte concentrate from three different donors (acquired from Blodcentralen, Lunds Universitetssjukhus, Lund, Sweden) through gradient centrifugation at 400×g for 40 minutes using Ficoll. Red blood cells (RBCs) were lysed using BD Pharmlyse buffer 10× diluted in milliQ water for 3 minutes at room temperature (RT). Cells were washed and the isolated PBMCs were pooled. Cell concentration was determined using a Scepter cell counter (Millipore Merck, MA. USA). Cells were diluted to 1×10⁷ cells/ml in PBS. Pooled PBMCs were split into 2 different tubes. Cells in tube 1 was stained with CFSE at 5 UM for 5 minutes (dark, RT). CFSE stained cells were washed with an equal volume of FBS to stop the reaction and washed again with PBS. Cells in tube two was left in PBS.

MSCs (donor 1, donor 2, donor 3, and AD-MSC control cells) were provided in 8 different tubes, two for each cell type (in different medias (M3 and M4). All cells (PBMCs and MSCs) were diluted in the two different cell culture media (M3 and M4) to a final concentration of 2*10⁶ cells/ml. Media was supplemented with anti-CD28 at 5 μg/ml. Cells were added to anti-CD3 (1 μg/well, 100 μl/well) coated U bottom cell culture plates according to the layout below, 200 μl/well in different PBMC:MSC ratios (1:10, 1:5, 1:2.5, 1:0 and 0:1) with a total amount of cells at 4*10⁵ cells/well.

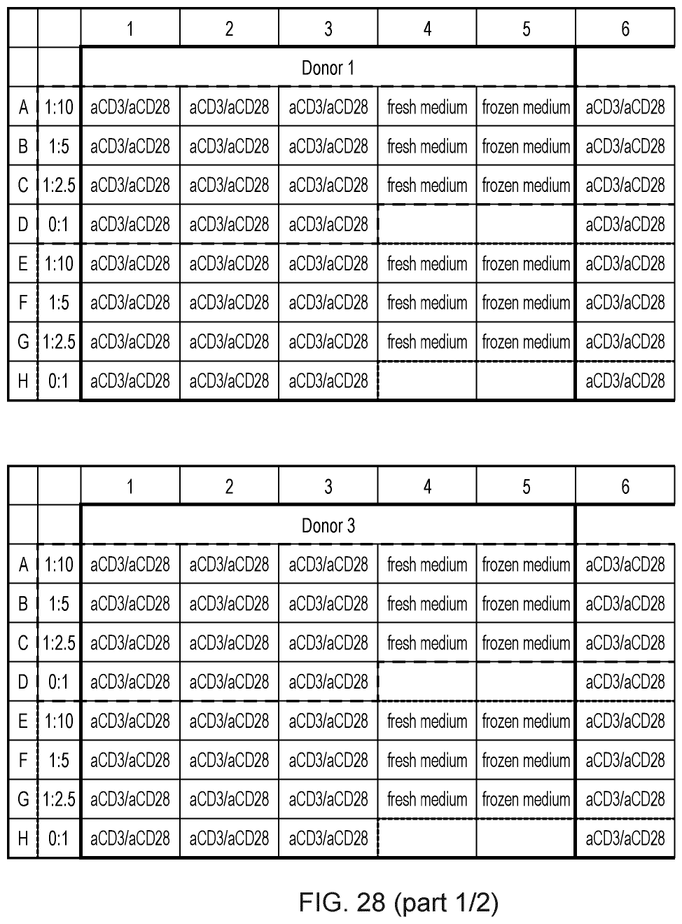

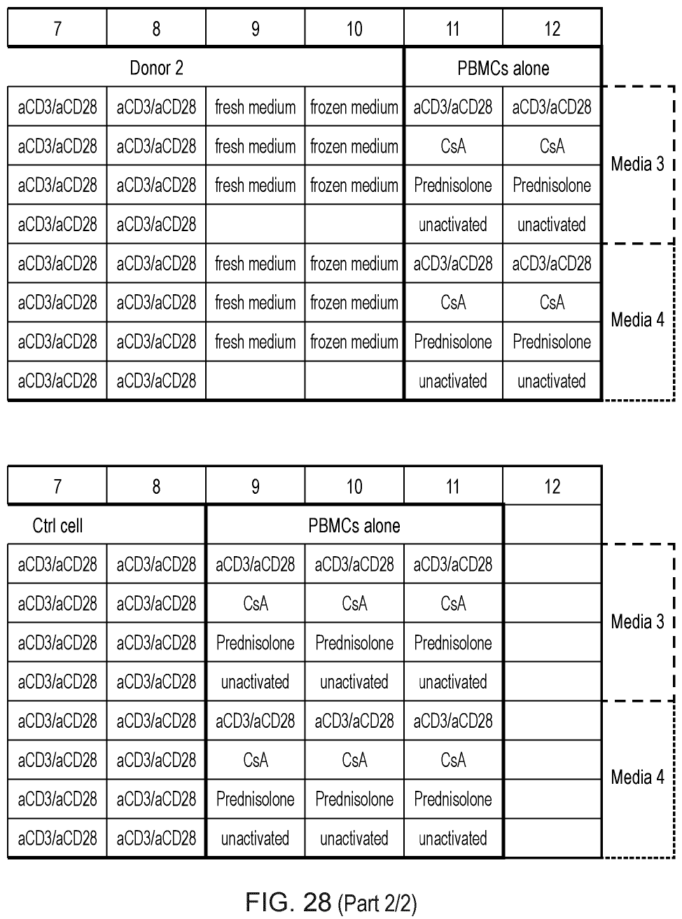

Included in the assay were also controls (stimulated and unstimulated PBMCs) and two reference drugs, e.g. cyclosporine (CsA) and Prednisolone (Pred). When plating controls, cells were spun down and diluted in M3 or M4 at 4*10⁶ cells/ml, with a total amount of cells at 4*10⁵ cells/well. 100 μl PBMCs were added per well. CsA was added at a final concentration of 10 μg/ml and Prednisolone was added at a final concentration of 125 nM (700 μl/well). Medium was added to unstimulated control wells (700 μl/well). Cells were incubated for 24 or 72 hours at 37° C., 5% CO2. See also FIG. 28.

2.3.2 Cell Composition after 24 Hours of Activation (FACS Analysis)

Following 24 hours of incubation, cells in FACS/Luminex plates were stained with antibodies detecting CD73, CD4, CD8, CD80, CD206, CD163 and PD-1 in staining buffer (PBS supplemented with 1% BSA). Briefly, cells were transferred to V bottom plates and centrifuged at 360×g for 2 min. Supernatant was transferred to a storage plate and put in −20° C. until analysis using Luminex. Cells were washed in PBS, centrifuged at 360×g for 2 min at 4° C. Supernatant was flicked off and antibodies against surface markers were added. Cells were incubated at +4° C. for 20 minutes (dark). After incubation, cells were washed 1× in PBS, centrifuged at 360×g for 2 min at 4° C. Cells were resuspended in PBS and acquired using the Attune Nxt flow cytometer. Compensation was performed using beads—AbC Total Compensation capture beads and negative beads. Since compensation was done using beads, FSC and SSC were changed before analysing samples and adjusted to cells. Lasers were not changed after compensation. 150 μl was analysed from each sample.

2.3.3 Proliferation and Cell Composition after 72 Hours of Activation (FACS Analysis)

After 72 hours of incubation, CFSE stained cells were stained with CD4, CD8, CD80, CD206 and CD163 in staining buffer. Briefly, cells were transferred to V bottom plates, centrifuged at 360×g for 2 min. Cells were washed in PBS, centrifuged at 360×g for 2 min at 4° C. Supernatant was flicked off and surface markers were added. Cells were incubated at +4° C. for 20 minutes. After incubation, cells were washed 1× in PBS, centrifuged at 360×g for 2 min at 4° C. Cells were resuspended in PBS and acquired using the Attune Nxt flow cytometer. Compensation was performed using beads—AbC Total Compensation capture beads and negative beads for surface markers. For CFSE compensation, newly CFSE stained cells were used. Since compensation was done using both beads and cells, FSC and SSC were changed and adjusted to beads or cells. Lasers were not changed after compensation.

2.3.4 Cytokine Analysis after 24 Hours of Activation (Luminex)

Supernatant (24 hours incubation) was analysed for cytokines using the 12-plex LXSAHM-12 Luminex kit. All reagents, standard and samples were prepared at room temperature and according to manufacturer's instruction. Briefly, 50 μl of standard or sample was added to wells. Microparticles were added to samples and standard and samples were incubated for 2 hours at room temperature on a horizontal orbital shaker (800 rpm). Plate was washed 3× in washing buffer using a magnetic plate. Biotin-antibody was added and incubated dark for 1 hour at room temperature on a horizontal orbital shaker (800 rpm). Plate was washed 3× in washing buffer using a magnetic plate. Streptavidin-PE was added to each well and incubated dark at room temperature on a horizontal orbital shaker (800 rpm) for 30 minutes. Plate was washed 3× in washing buffer using a magnetic plate. Microparticles were resuspended in 100 μl washing buffer, incubated for 2 minutes on a horizontal orbital shaker (800 rpm). Samples were analysed on a Bio-Rad Luminex analyzer.

### 2.4 Acquisition and Analysis

Graphs were performed using Prism 8 for Mac OS X (GraphPad Software, San Diego, CA, USA). Results are presented as mean values±SEM, if not otherwise stated. Dot plots were acquired from analysis using FlowJo v10.6.1 for Mac (BD, New Jersey, USA). The inhibitory effect of MSCs on PBMC proliferation was calculated according to the two formulas below:

$$Coculture norm prolif ( \% ) = \frac{Coculture prolif}{Stimulated single PBMC culture prolif}$$

$$Inhibitory effect = 100 - coculture normalized prolif$$

Ref: Optimisation of a potency assay for the assessment of immunomodulative potential of clinical grade multipotent mesenchymal stromal cells, Irene Oliver-Vila, Received: 4 Oct. 2017/Accepted: 29 Dec. 2017/Published online: 10 Jan. 2018 6 Springer Science+Business Media B.V., part of Springer Nature 2018, Cytotechnology (2018) 70:31-44 https://doi.org/10.1007/s10616-017-0186-0

### 3. Results

3.1 Cell Composition after 24 Hours of Activations (FACS Analysis)

### 3.1.1 Gating Strategy

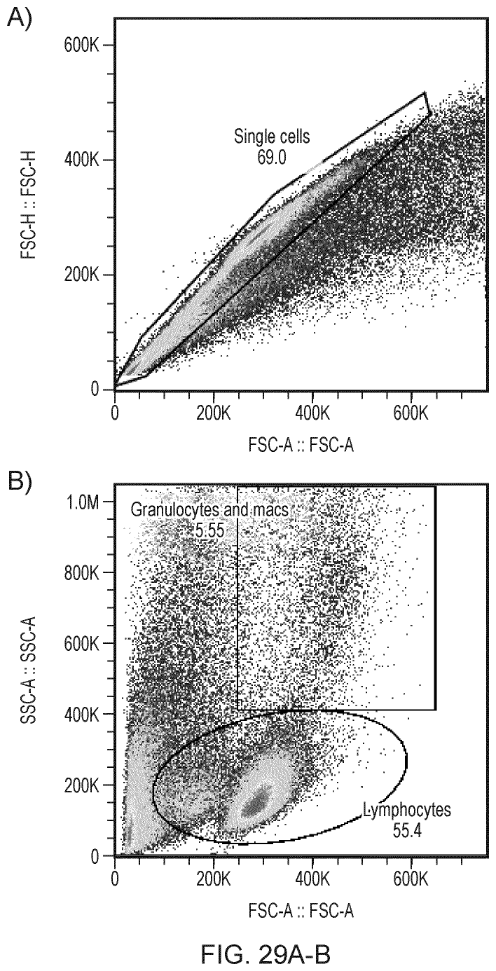

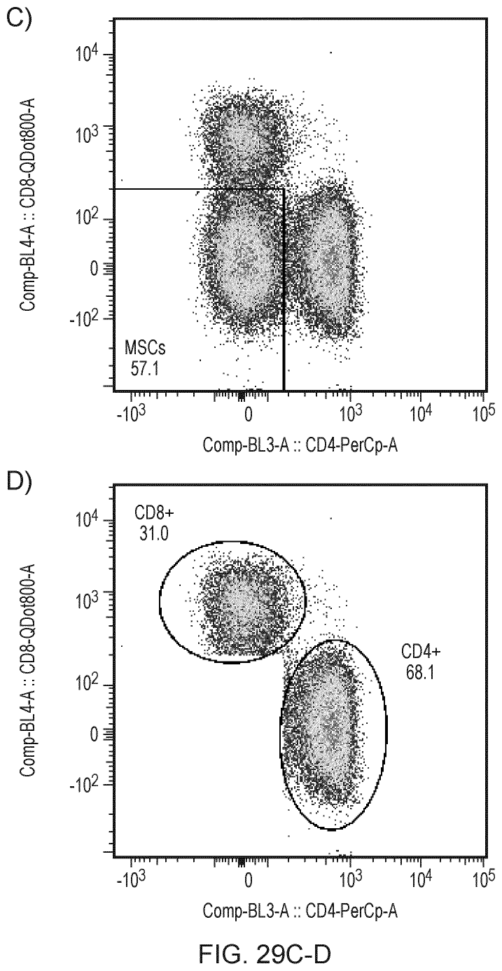

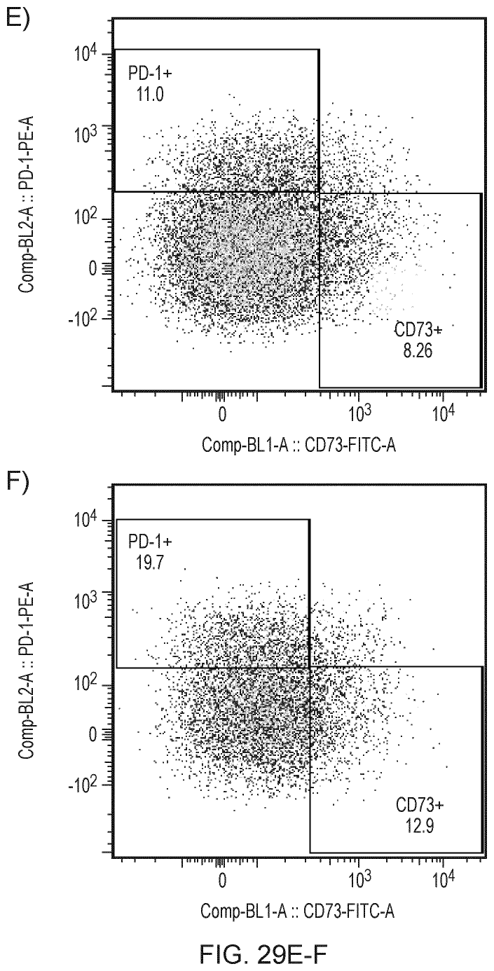

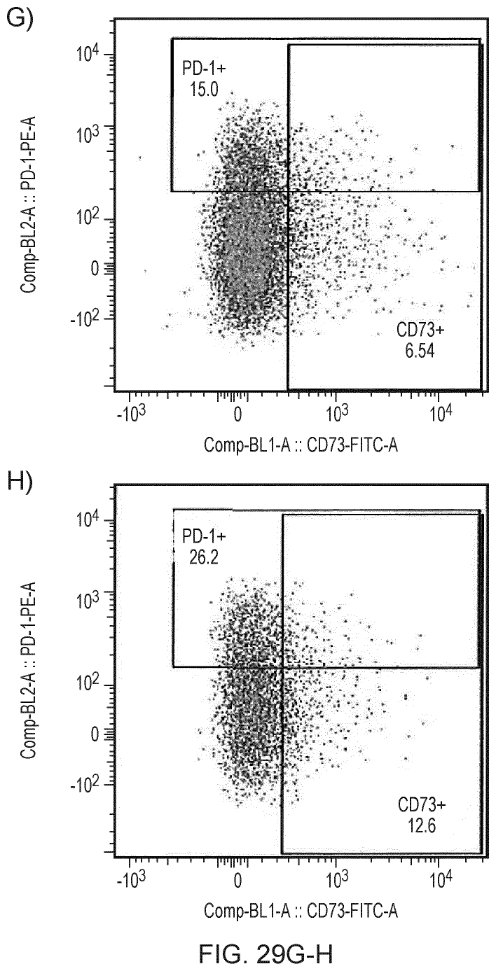

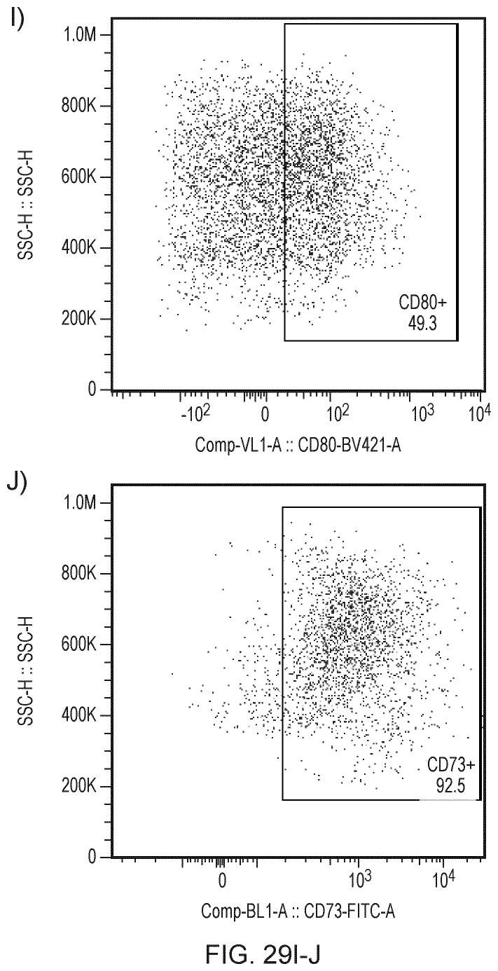

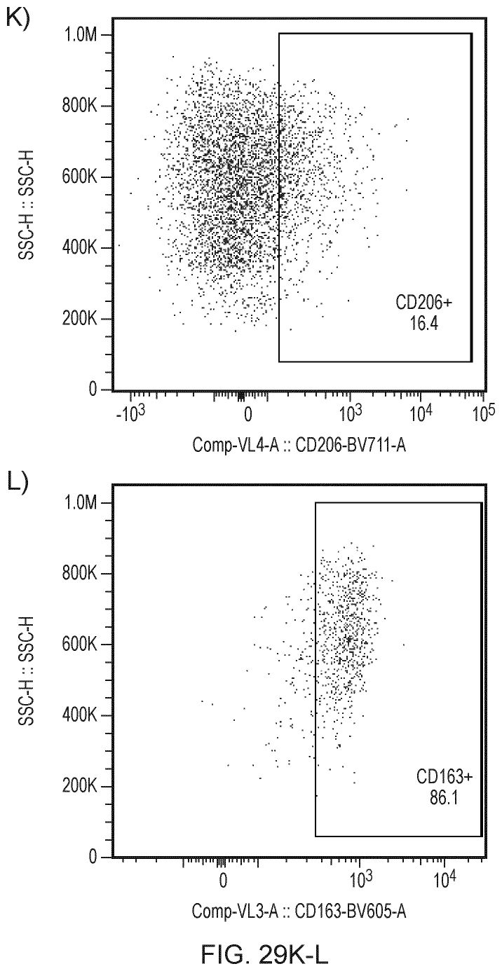

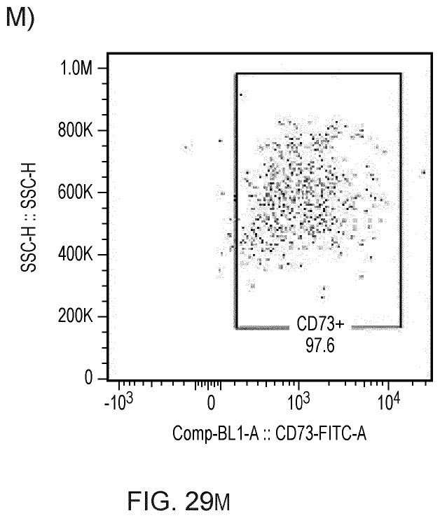

See FIG. 29

### 3.1.2 Representative Plots of Gating on Different Ratios

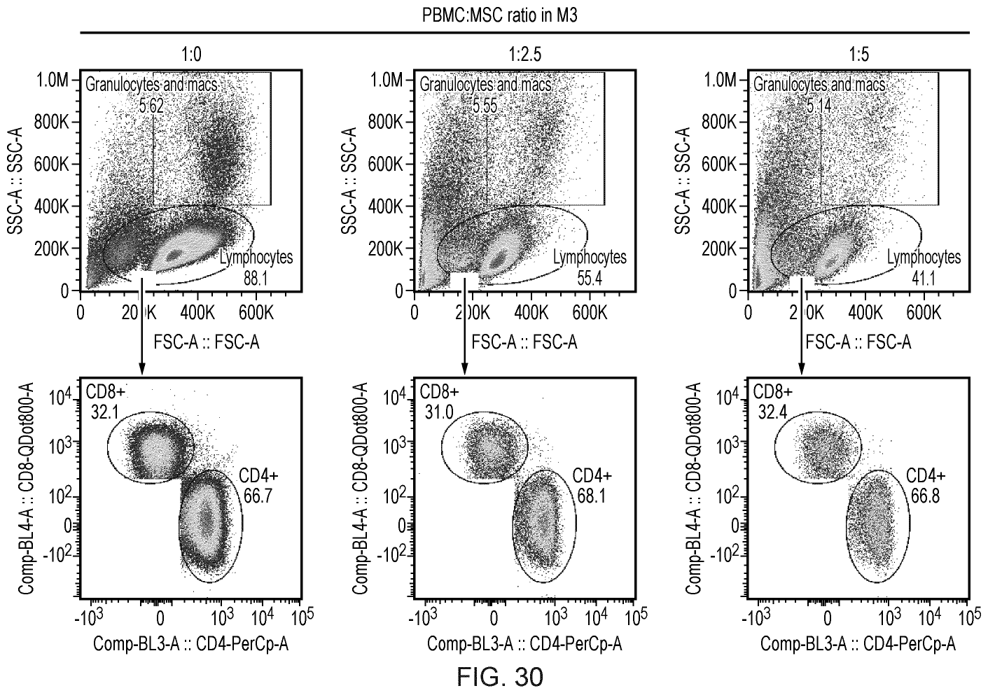

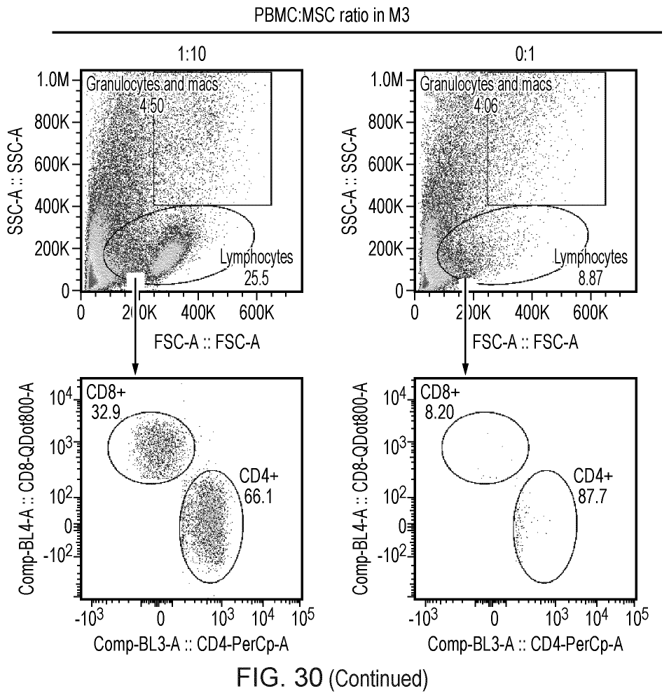

See FIG. 30

3.1.3 Results Cell Composition after 24 Hours of Activation—Shown for M3
Th (T helper cells) are PD-1+ cells among CD4+ cells.
Treg (Regulatory T cells) are CD73+ cells among CD4+ cells.
Tc (Cytotoxic T cells) are PD-1+ cells among CD8+ cells.
Teff (Effector T cells) are CD73+ cells among CD8+ cells.
M1 (M1 macrophages)—Macrophages that encourage inflammation.
M2 (M2 macrophages)—Macrophages that decrease inflammation and encourage tissue repair.

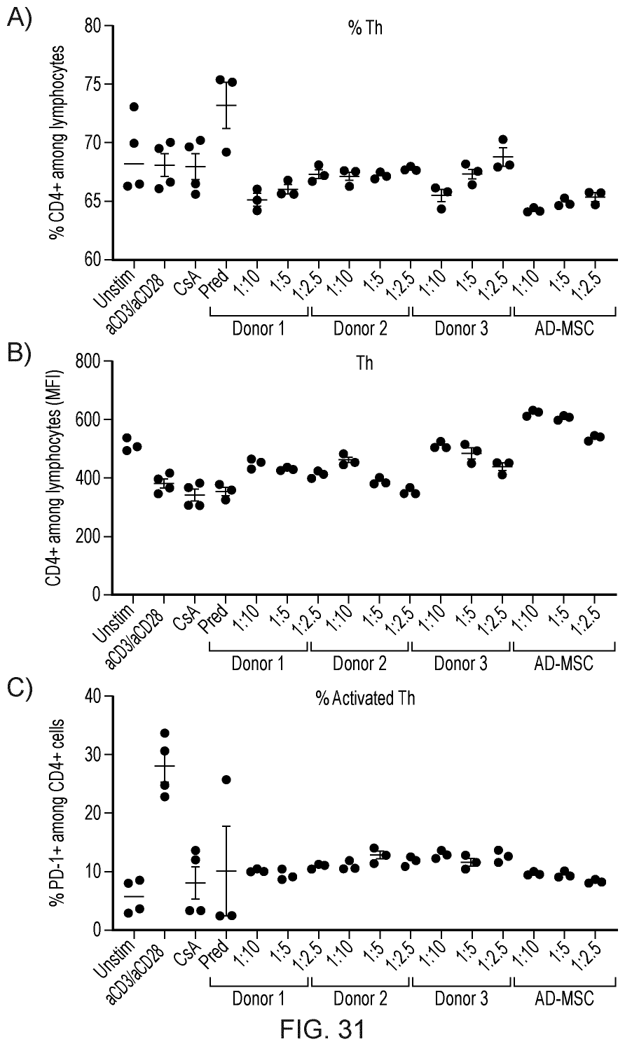

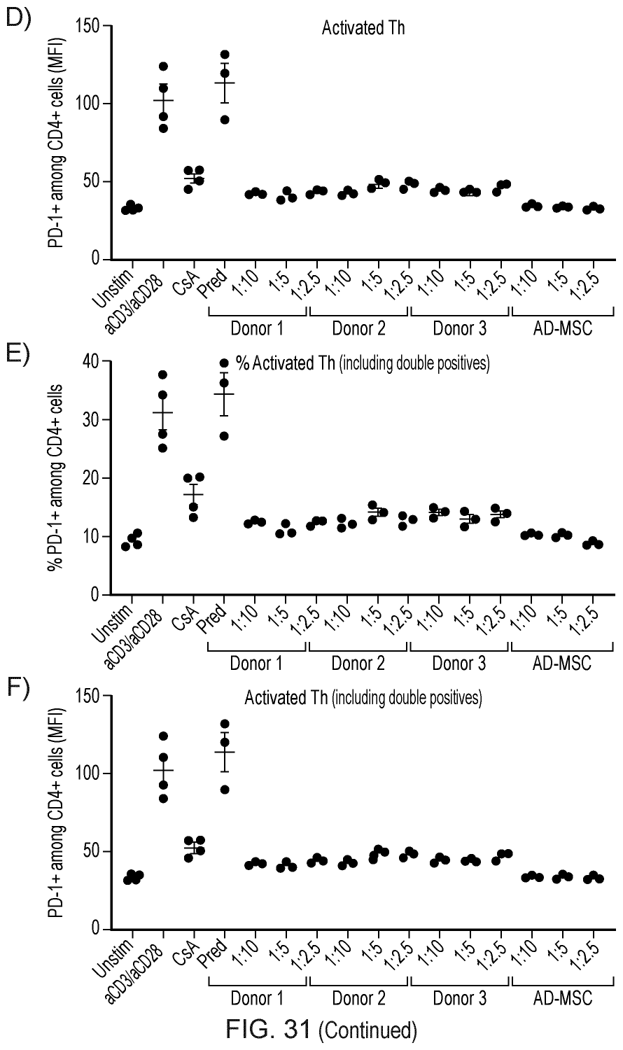

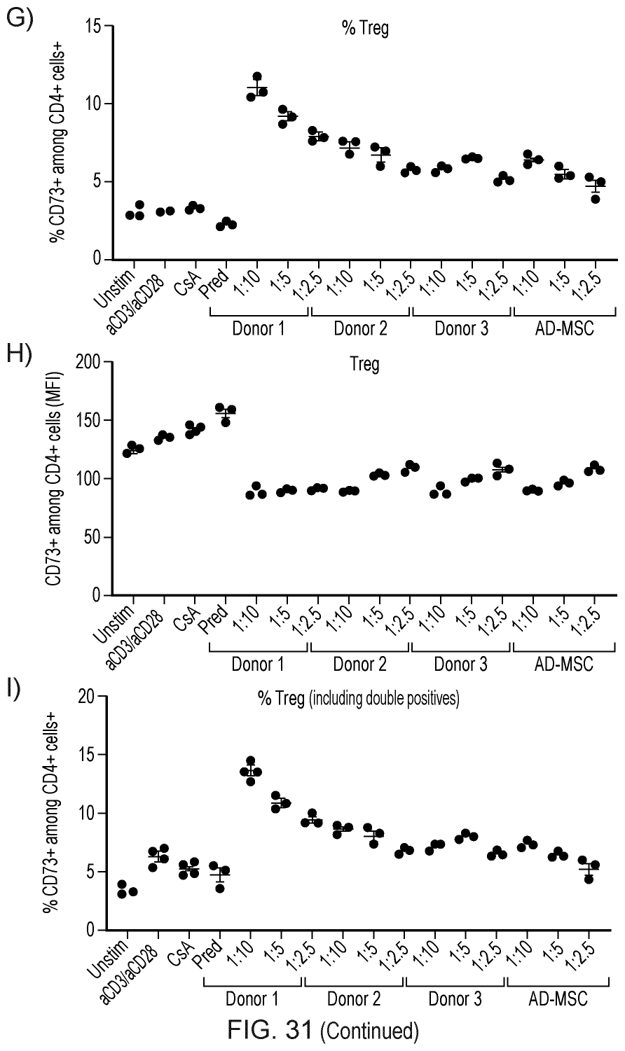

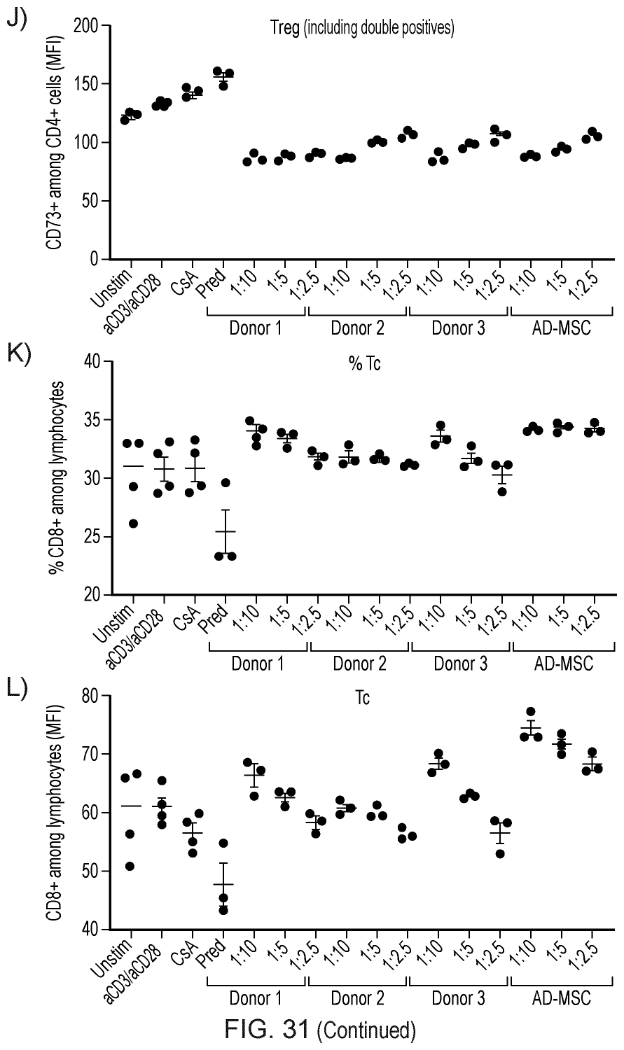

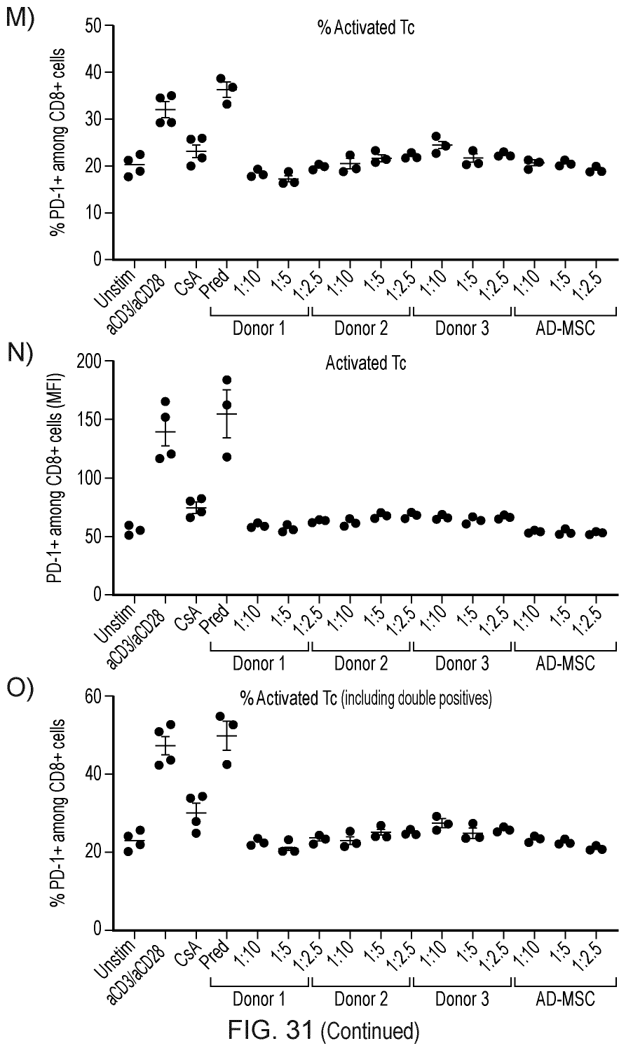

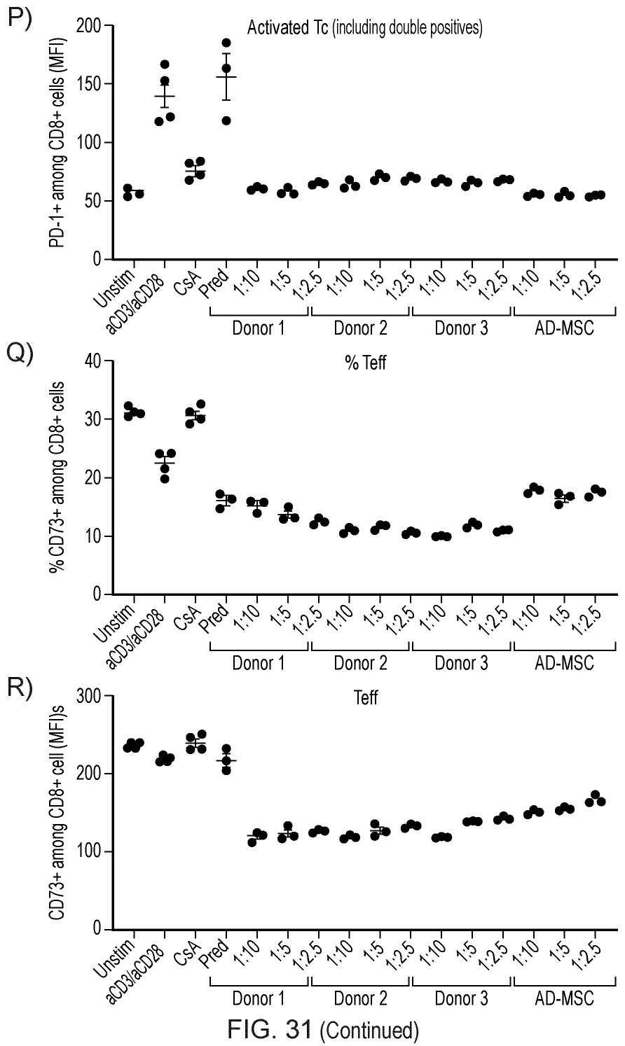

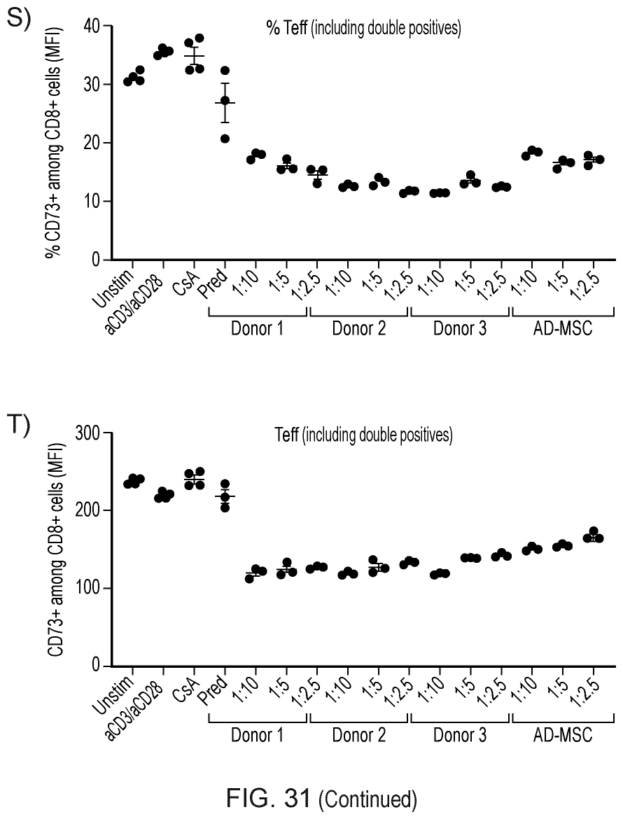

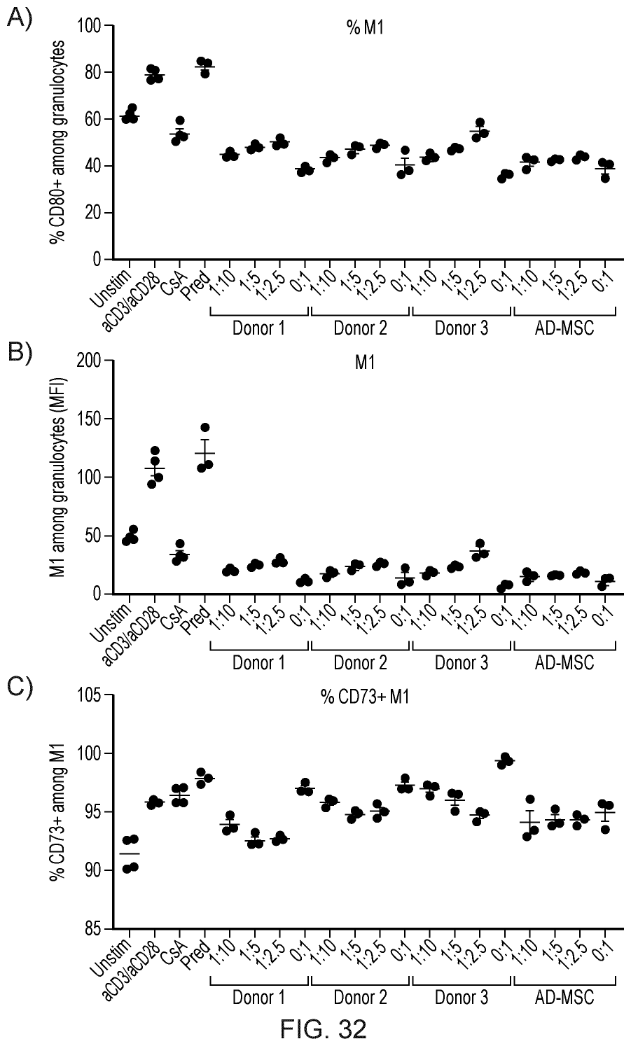

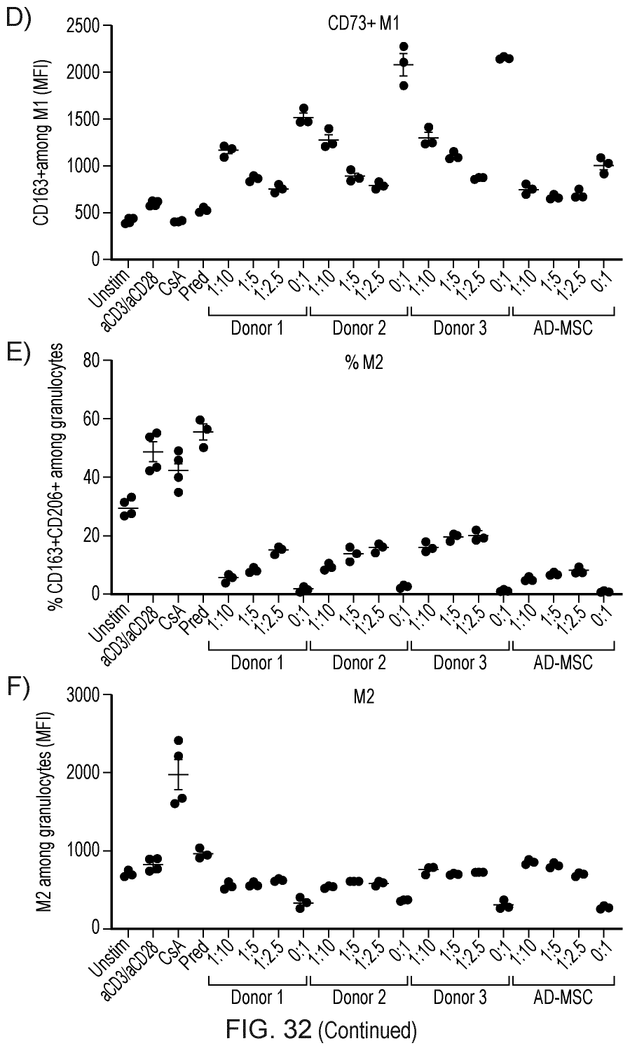

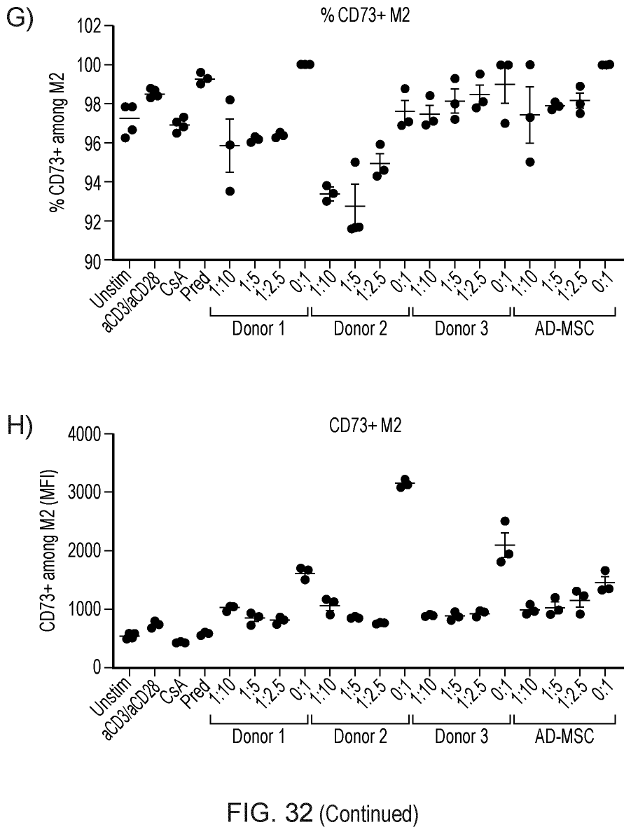

See FIG. 31 and FIG. 32

3.2 Proliferation and Cell Composition after 72 Hours of Activation (FACS Analysis after CFSE Labeling)

CFSE labeling of PBMCs was performed before coculture with MSCs, therefore all CFSE positive cells should be PBMCs.

### 3.2.1 Gating Strategy

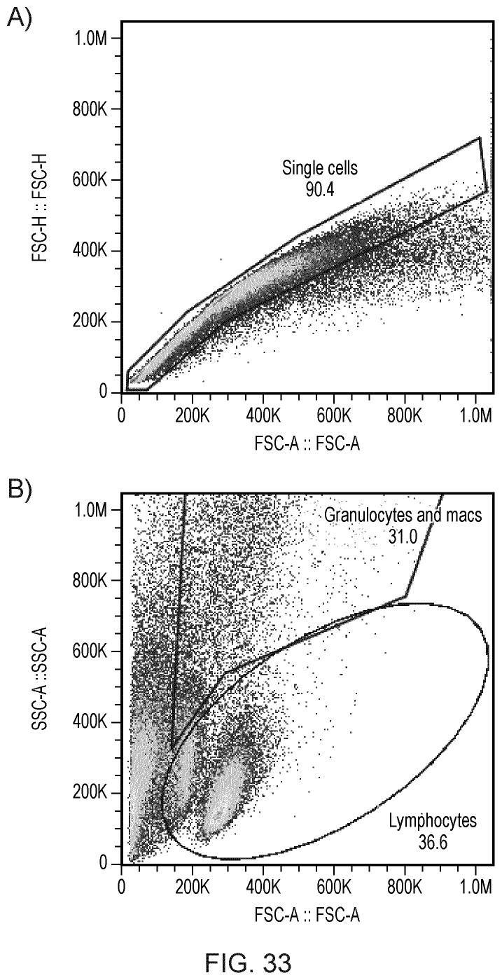

See FIG. 33

### 3.2.2 Gating Strategy for Cell Proliferation (CFSE)

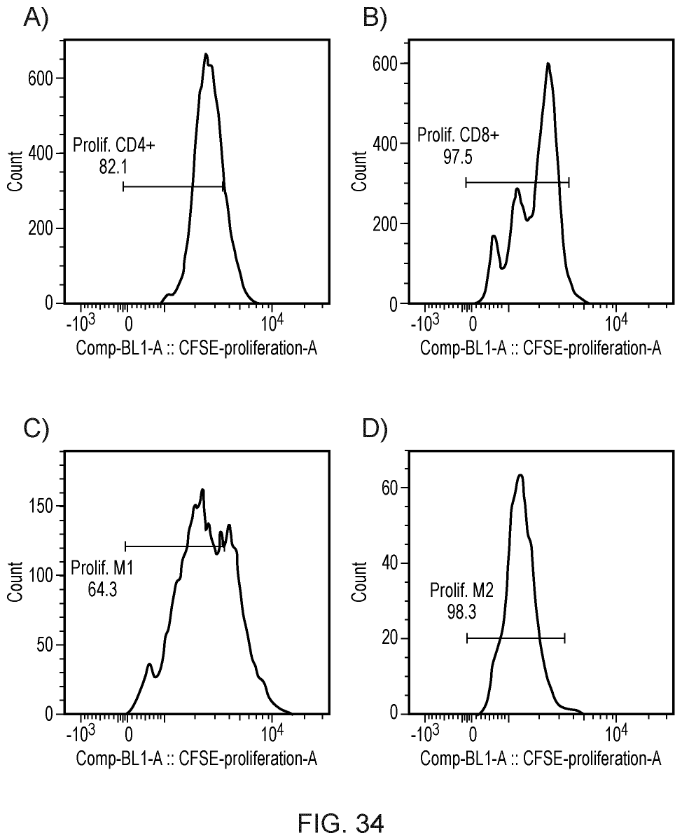

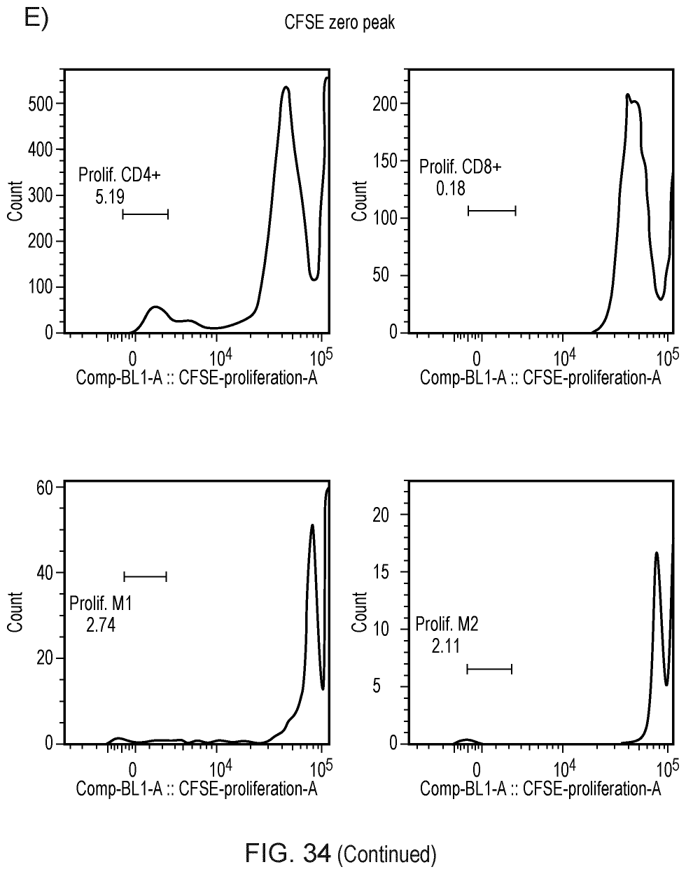

See FIG. 34A-E

3.2.3 Staining with CFSE

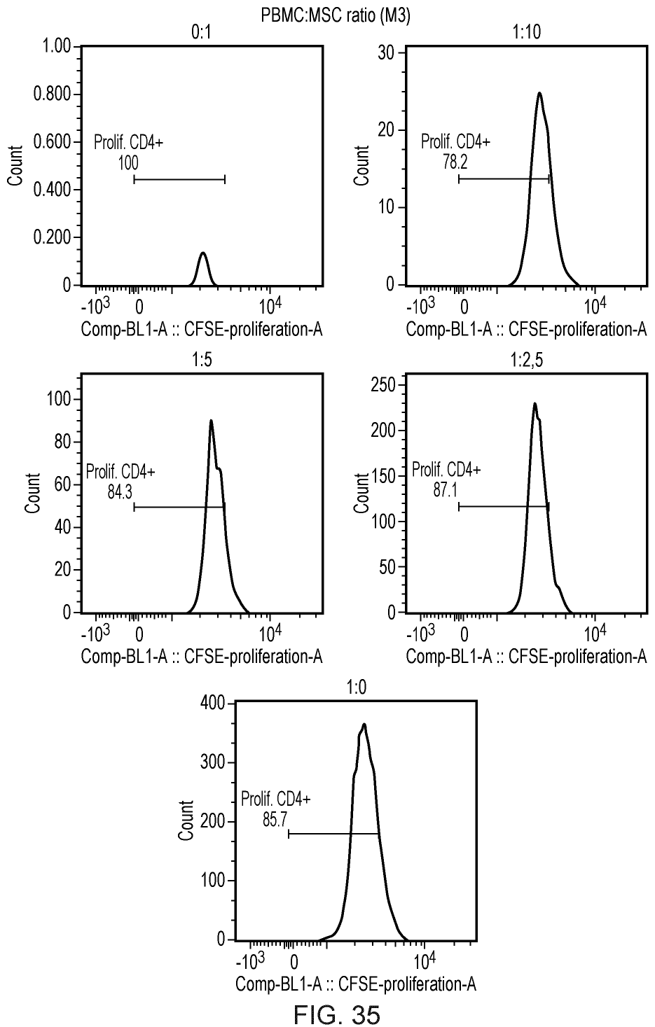

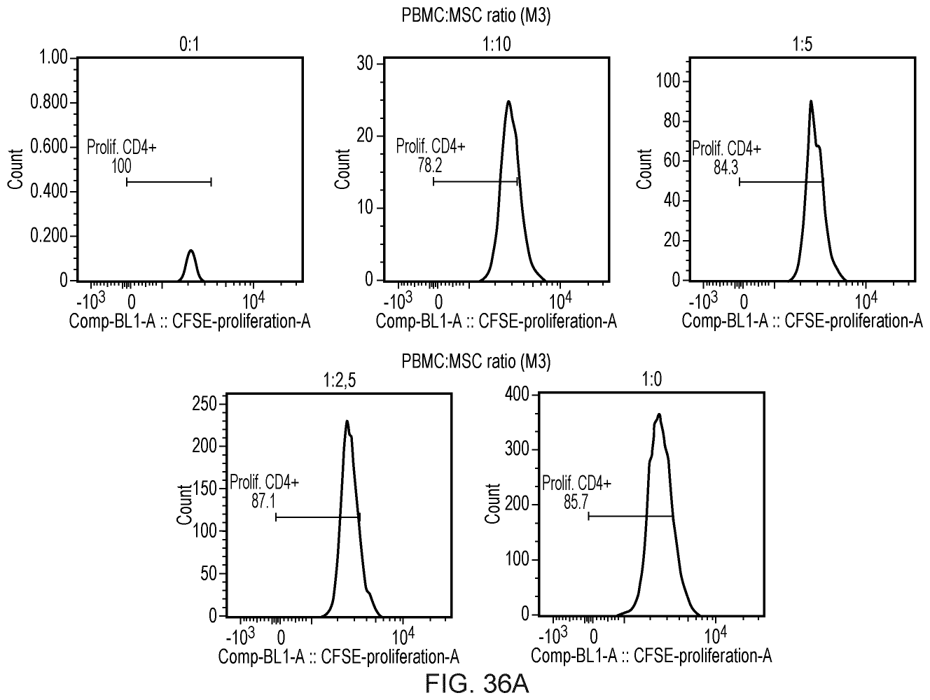

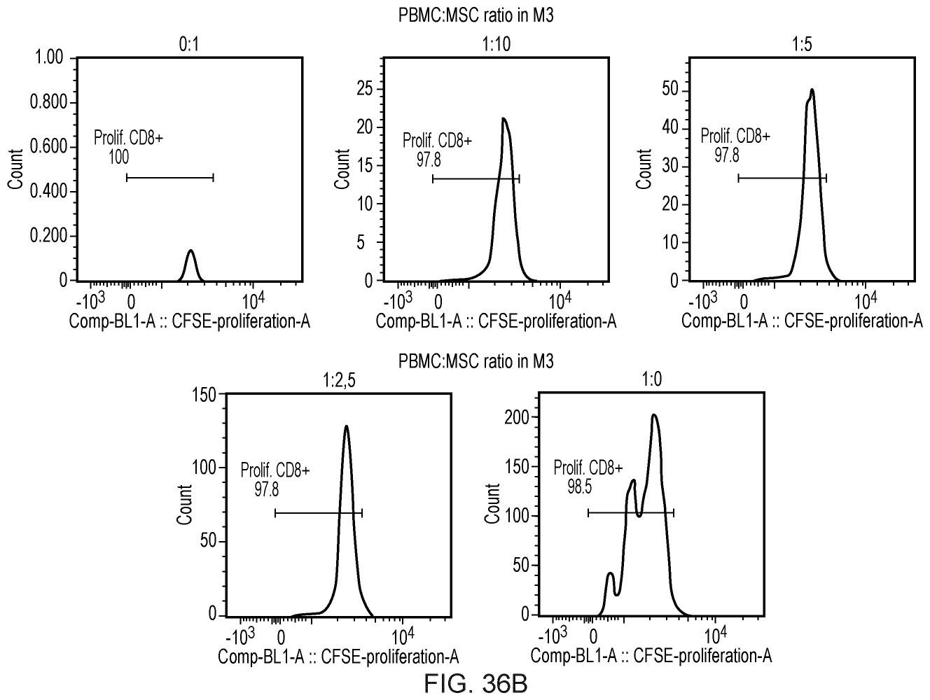

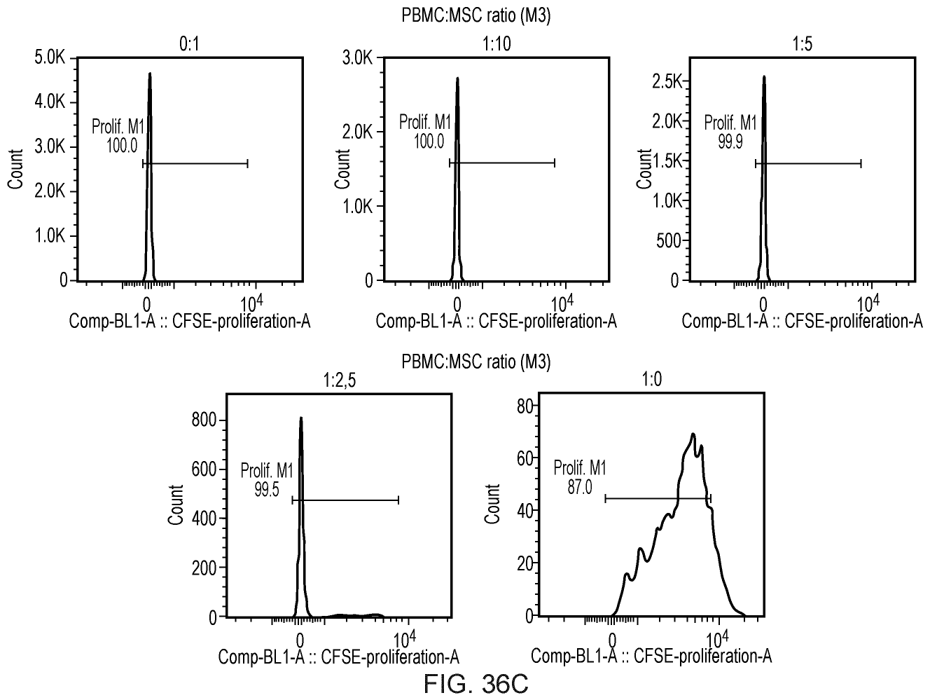

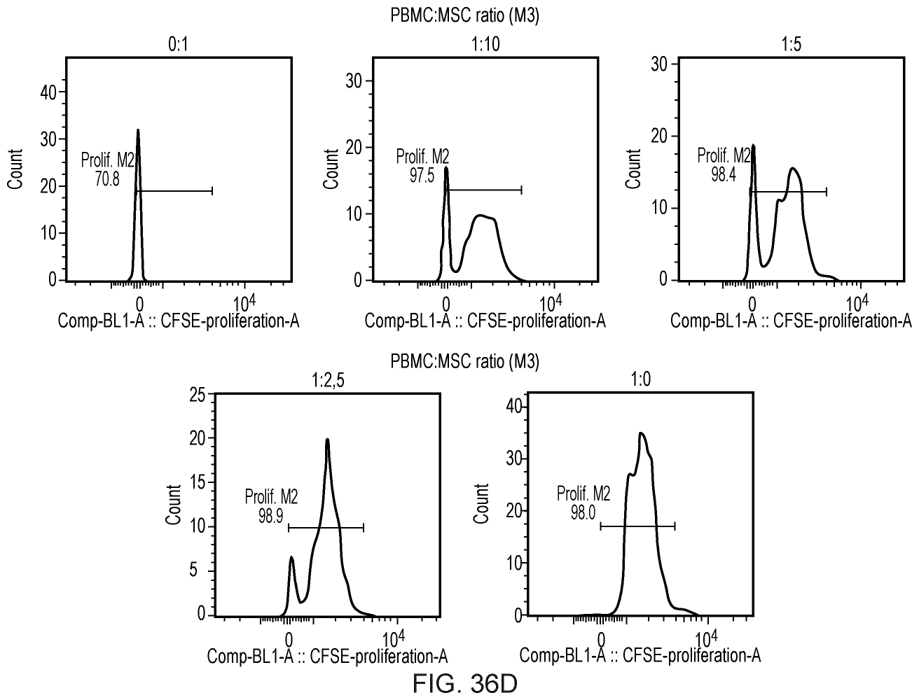

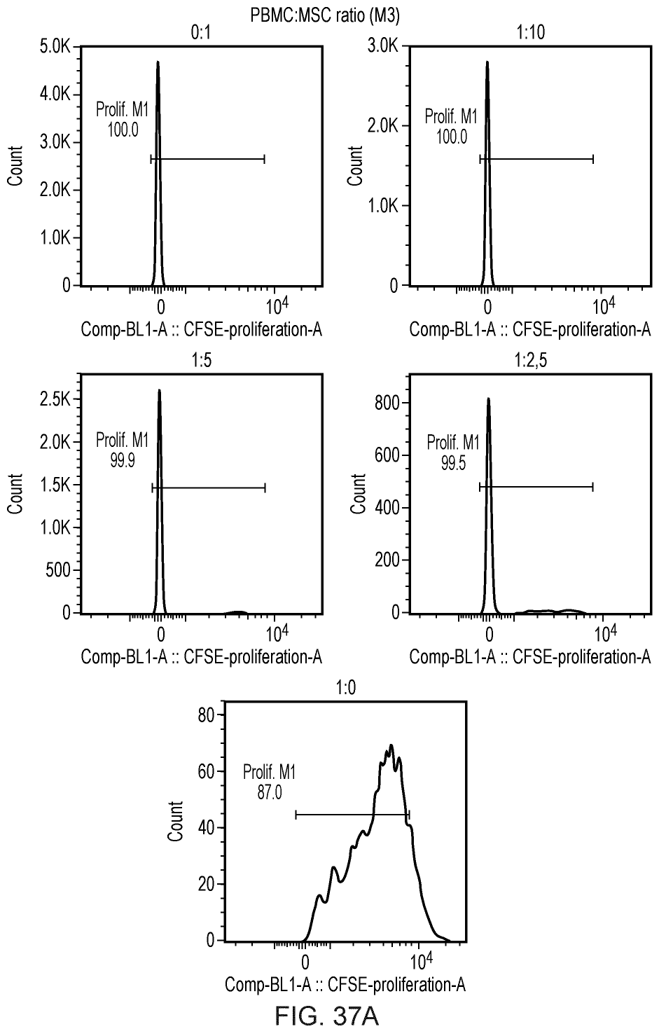

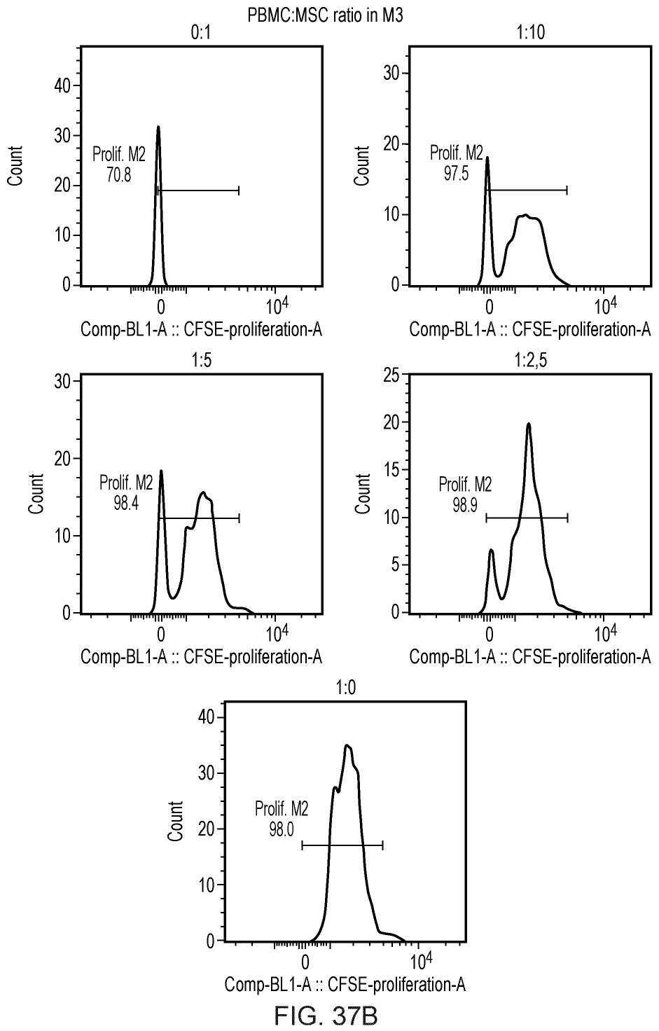

See FIG. 35-37A-B

3.2.4 Results—Proliferation and Cell Composition after 72 Hours of Activation (FACS Analysis after CFSE Labeling)—Shown for Media 3

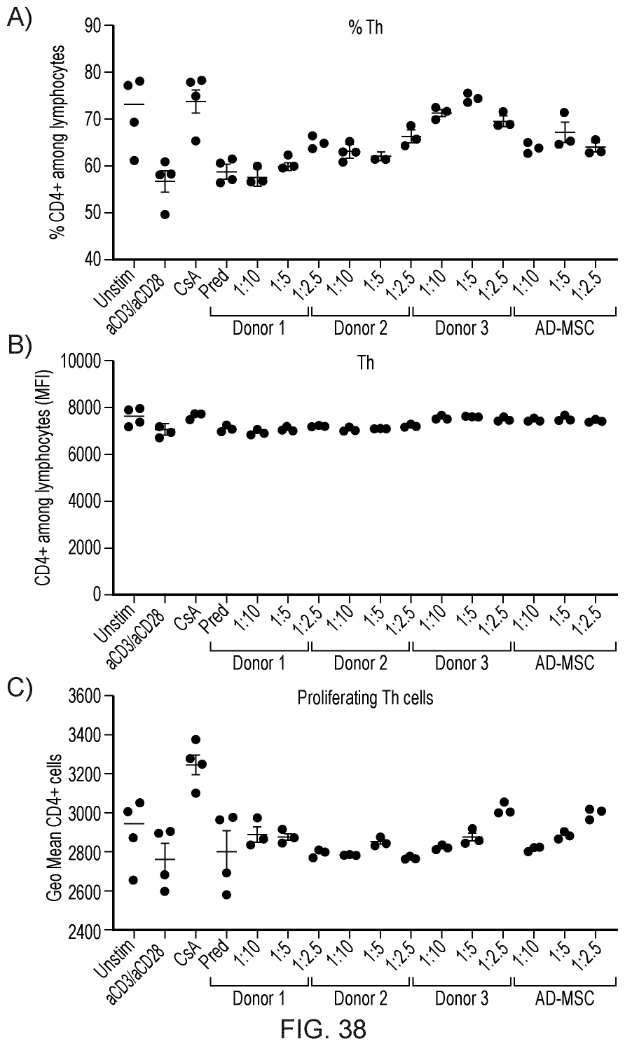

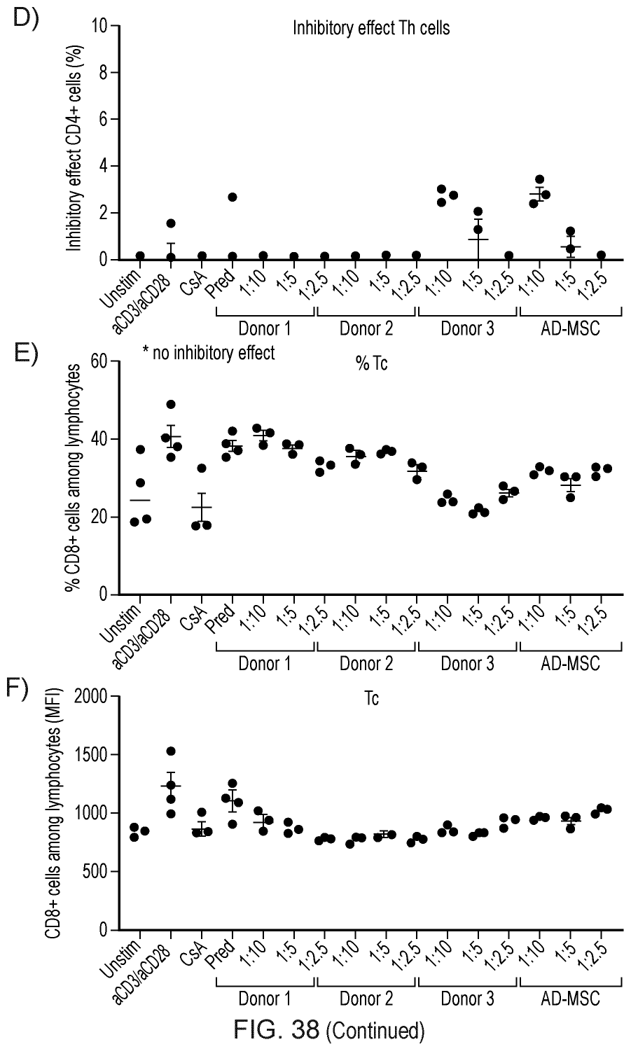

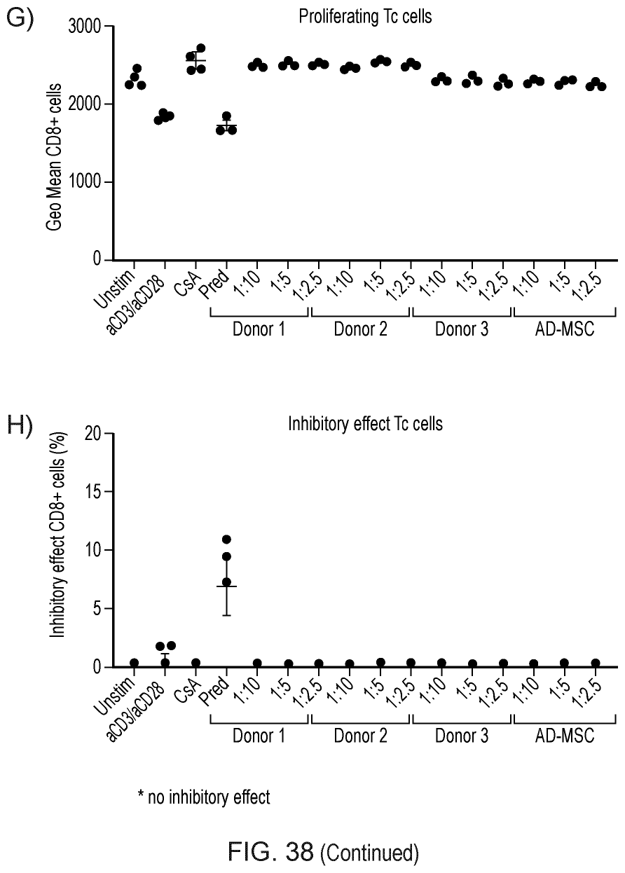

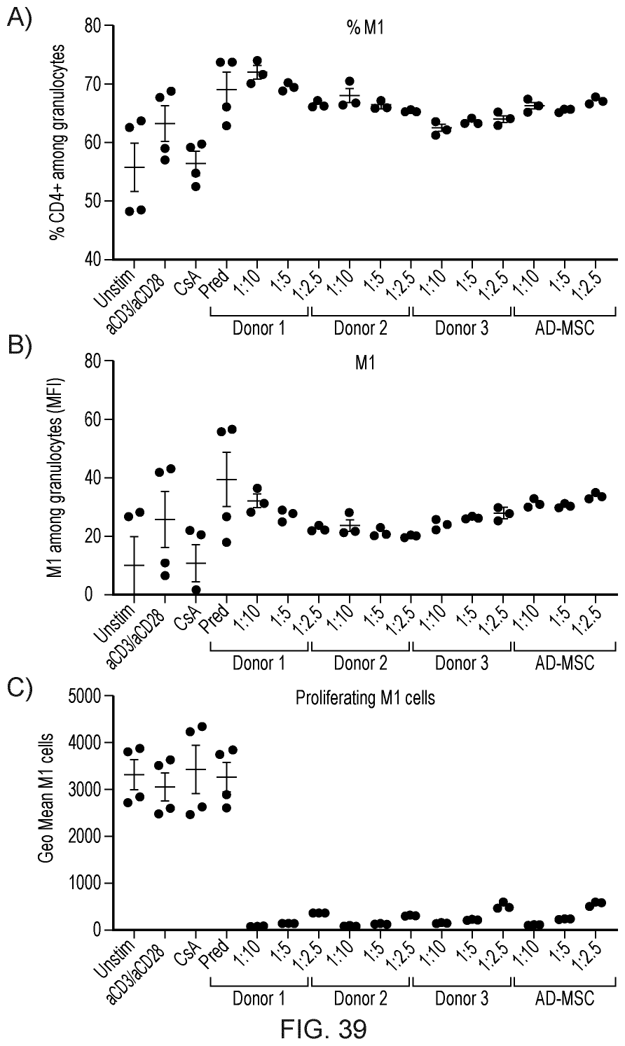

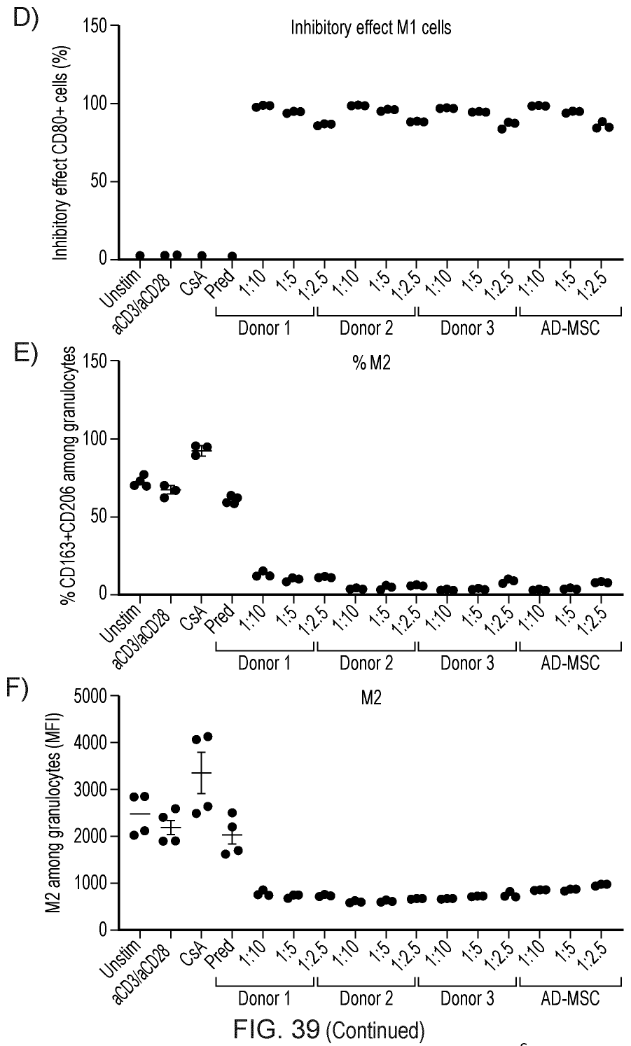

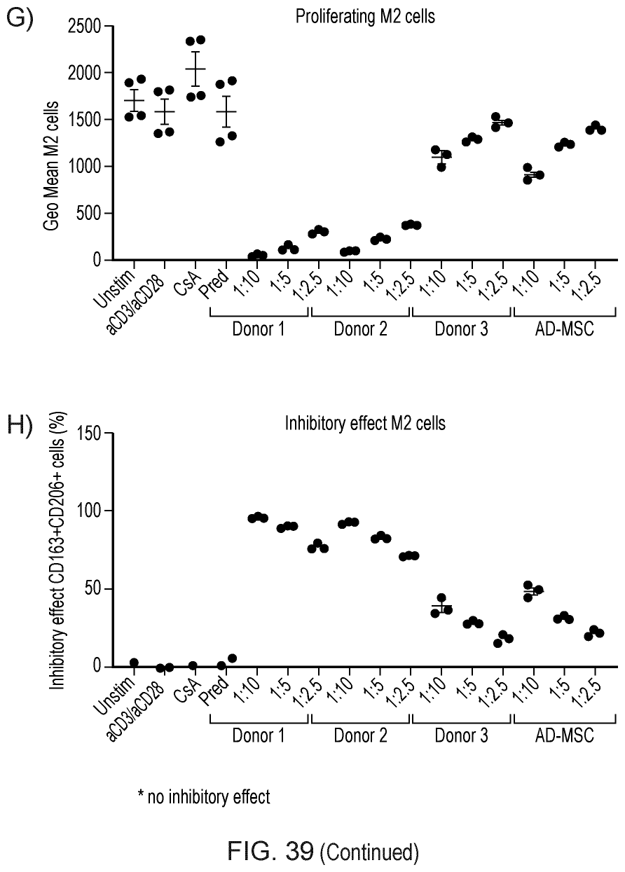

See FIG. 38-39

3.3 Cytokine Analysis after 24 Hours of Activation (Luminex)—Shown for Media 3

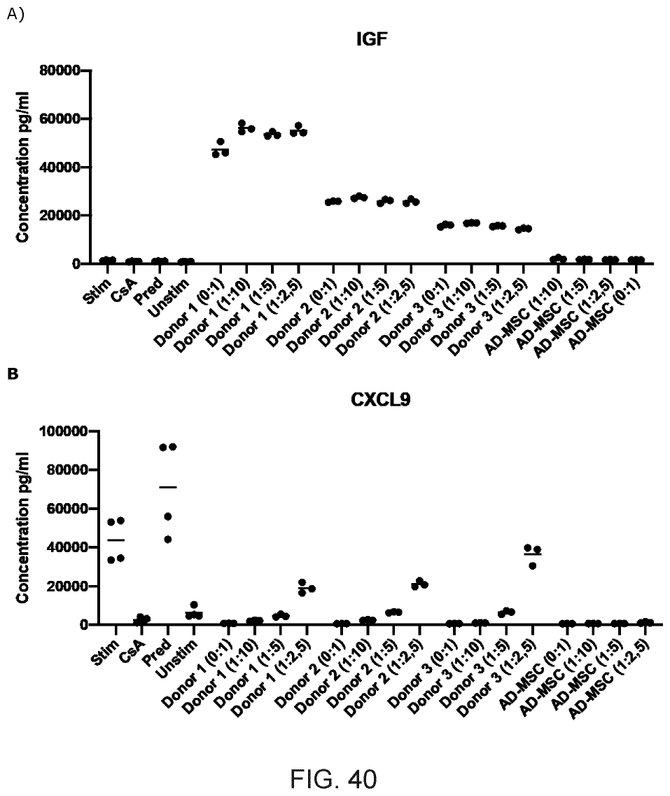

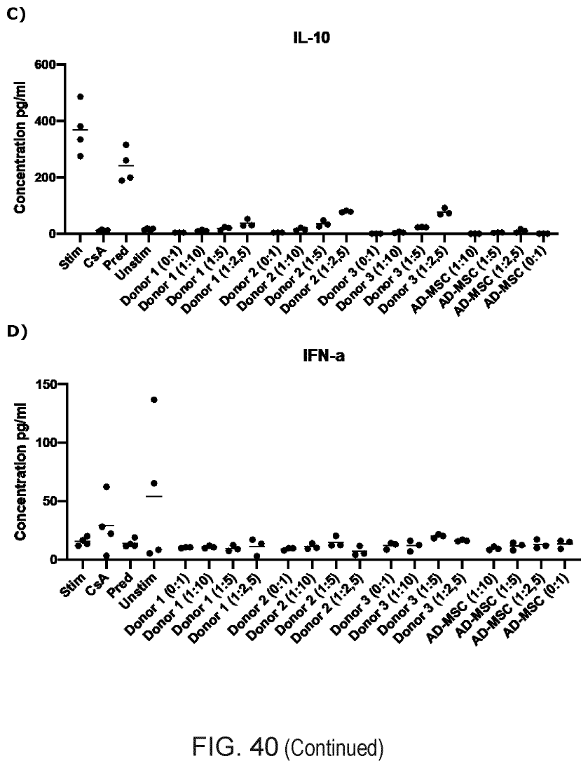

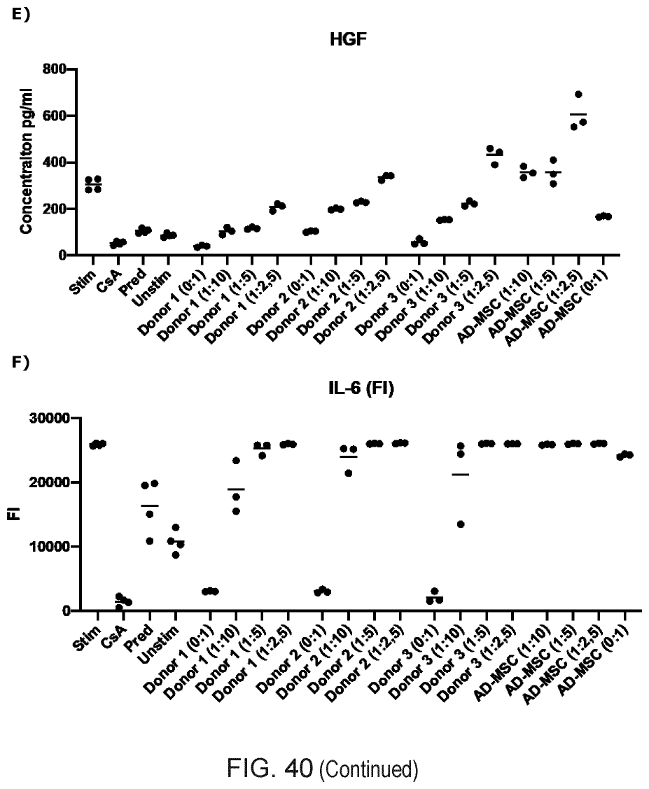

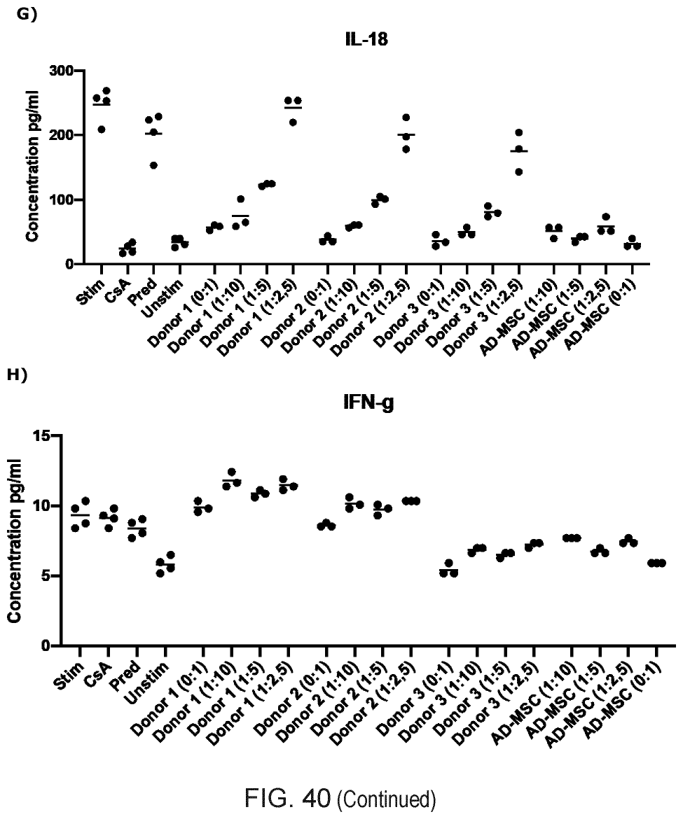

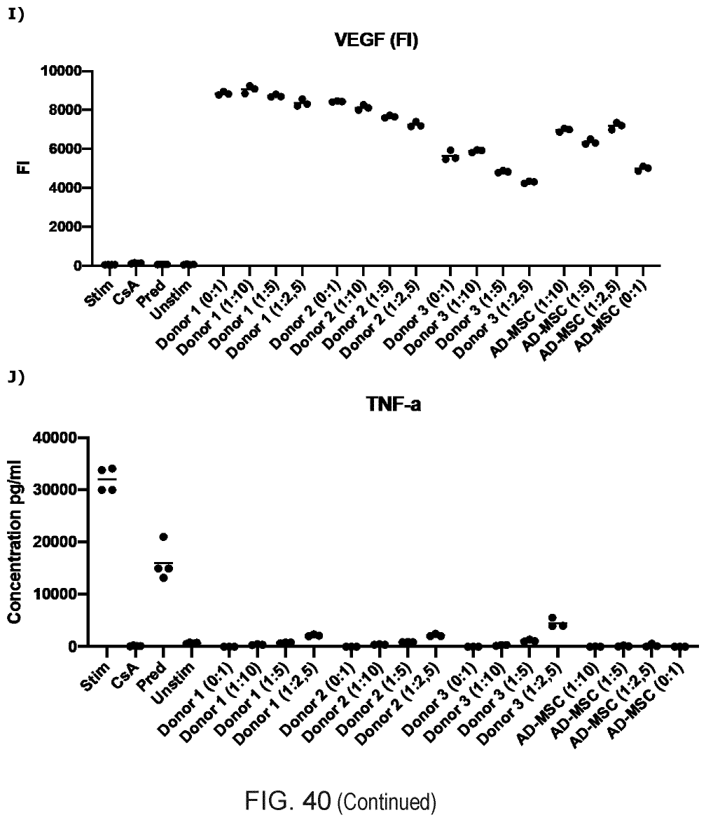

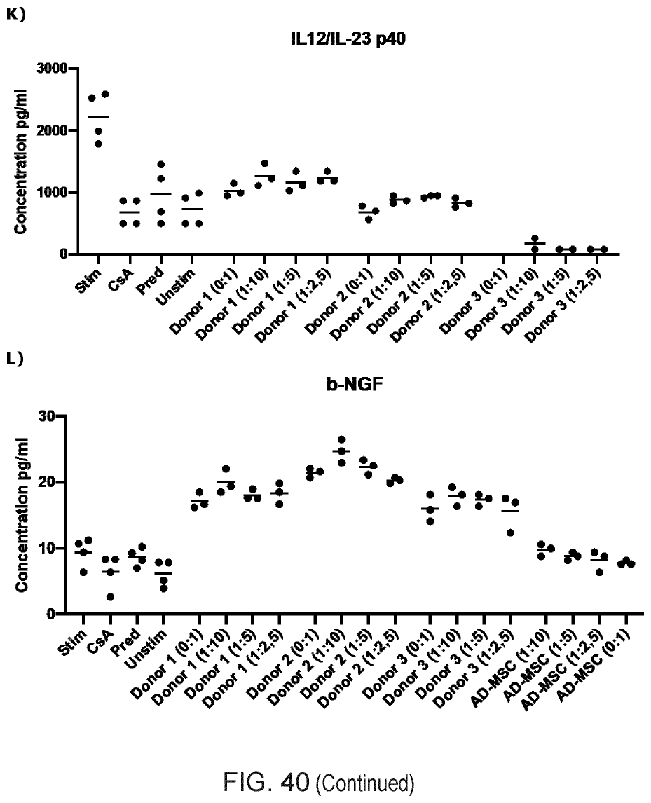

See FIG. 40

### 3.4 Luminex Analysis—CM Compared to Controls in Media 3

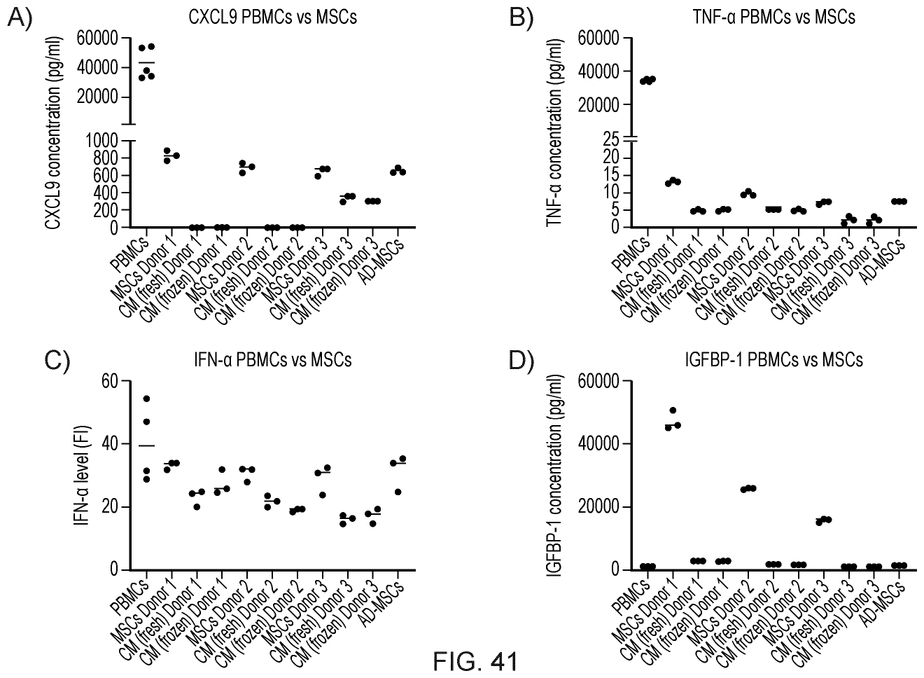

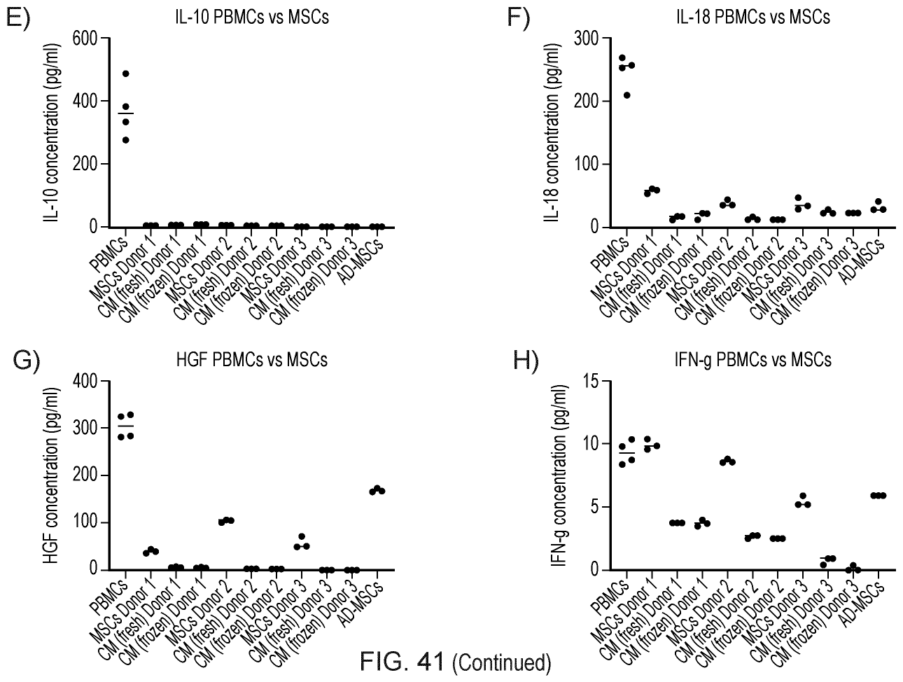

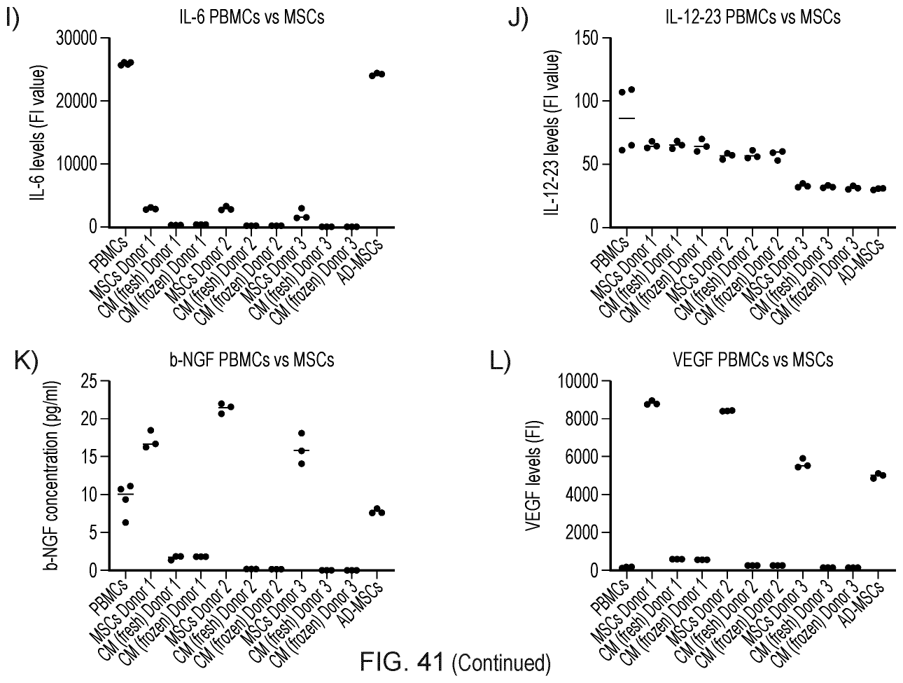

See FIG. 41

### 4. Conclusion

In this study, novel test items effect was evaluated on T cell activation and macrophage activation/polarization in human PBMCs. PBMCs were isolated from leucocyte concentrate from three different donors. Pooled PBMCs were either stained with CFSE at 5 μM for 5 minutes and added to MSCs or directly added to MSCs. Pooling of cells were performed to get a mean value from three donors but also generates a mixed lymphocyte reaction. PBMCs and MSCs were diluted in two different cell culture media (M3 and M4) at different PBMC:MSC ratios. Cell populations were analysed using flow cytometry after 24 or 72 hours. CFSE proliferation was analysed after 72 hours and cytokine levels in supernatant were analysed after 24 hours. No cut off on cell numbers were included in this analysis. Back gating was performed to verify cell populations for cells stained for cellular marker expression. For cells stained with CFSE the gates were narrowed due to unspecific leakage from channel BL1 into other channels.

PBMC:MSC ratios used in this experiment were: 1:0, 1:2.5, 1:5, 1:10 and 0:1. Thus, more MSCs than PBMCs were added per well.

PBMC:MSC ratio 0:1 in FIG. 30 shows almost no T cells or macrophages (PBMCs not added to sample) and FACS results can therefore not be compared with other ratios.

According to results in FIG. 31, showing T cell activation status after co-culturing PBMCs with MSCs or reference drugs, co-culture with PBMCs and MSCs show no effect on the composition of T helper cells (% CD4+ lymphocytes or MFI of CD4+ lymphocytes) after 24 hours of incubation (FIG. 31A, 31B). However, results show that there is a lower percentage (8-13%) of activated T helper cells (PD-1+CD4+ lymphocytes) compared with control (23-33%) after 24 hours of incubation at all PBMC:MSC ratios (FIG. 31C, 31D, 31E, 31F). The percentage of T regs (CD73+CD4+ lymphocytes) were higher than control after co-culturing PBMCs and MSCs at all ratios (5-10% vs 2-3%), but the expression level (MFI) of T regs were lower compared to control (FIG. 31G, 31H, 31I, 31J). The number of T regs in ratio 0:1 is approximately 80 cells, indicating that results after co-culturing PBMCs with MSCs are correct. Results show no effect on cytotoxic T cell composition or expression level of CD8 when PBMCs were co-cultured with MSCs (% CD8+ lymphocytes or MFI of CD8+ lymphocytes) after 24 hours of incubation (FIG. 31K, 31L). However, the percentage of activated cytotoxic T cells (% PD-1+CD8+ lymphocytes) is lower (16-25%) at all PBMC:MSC ratios compared with control (29-35%) (FIG. 31M, 31O). Similarly, MFI values decreased from 120-170 in control to 50-60 in all PBMC:MSC ratios (FIG. 31N, 31P). The expression level of T effector cells (CD73+CD8+ lymphocytes) is also lower at all PBMC:MSC ratios (FIG. 31Q, 31R, 31S, 31T) compared with control (10-15% vs 23%).

In summary, co-culture of PBMCs and MSCs results in fewer activated T helper cells, more regulatory T cells, fewer activated cytotoxic T cells, and fewer effector T cells compared to PBMC controls.

According to results in FIG. 32, showing macrophage activation status, co-culture of PBMCs with MSCs results in fewer activated macrophages. The percentage of M1 macrophages (CD80+ granulocytes/macrophages) drops from 80% to 40% while MFI values drop from 120 to less than 25 (FIG. 32A, 32B). At the same time the percentage of M2 macrophages (CD163+CD206+ granulocytes/macrophages) drops from 50% to 5-20% while MFI values drop from 2000 to less than 750 (FIG. 32E, 32F). CD73 was almost universally expressed on activated (M1 and M2) macrophages (FIG. 32C, 32G). However, an increased expression intensity of CD73 can be seen in both M1 (MFI 750-1250 of CD80+ macrophages compared to 600 in controls) and M2 (MFI of 800-1000 of CD163+CD206+ macrophages compared to 750 in controls) after co-culture of PBMCs and MSCs (FIG. 32D, 32H).

Assay controls (cell composition): Prednisolone treated PBMCs show lower levels of % cytotoxic T cells (FIG. 30G) and higher levels of % T helper cells (FIG. 30A), but only slightly inhibits T cell activation (FIG. 30C, 30D, 30I, 30J). CsA treated PBMCs show a lower expression level of activated T helper cells (FIG. 30C, 30D) and activated cytotoxic T cells (FIG. 30I, 30J). However, the inhibition of T cell activation by CsA was not as good as that seen with co-culture with MSCs (FIG. 30C, 30D, 30I, 30J).

Prednisolone did not have an effect on macrophage cell composition (FIG. 32). CsA treated PBMCs show a lower expression level of M1 macrophages (FIG. 32A, 32B), but not with as large an effect on macrophage activation as MSCs. CsA also showed a shift towards M2 macrophage expression (FIG. 32E, 32F) and a lower expression level of CD73+M1 (FIG. 32C, 32D) and CD73+M2 (FIG. 32G, 32H).

In summary, co-culture of PBMCs and MSCs result in fewer activated macrophages, both of the M1 and the M2 subtypes, compared to PBMC controls. Co-culture with PBMCs and MSCs almost remove the granulocyte population indicating a reverse effect on an activated immune response. Prednisolone treated cells show no effect on the M1 or M2 expression levels although, CsA treated cells show a shift towards M2.

According to results in FIG. 38, a co-culture with PBMCs and MSCs show no effect on T helper cells. Although, a small inhibitory effect can be seen on cytotoxic T cells after 72 hours of incubation. No effect can be seen on T cell proliferation when using a co-culture with PBMCs and MSCs. As results show in FIG. 39, a co-culture with PBMCs and MSCs show a strong inhibitory effect on M2 expression levels but no effect on M1 expression levels. A strong inhibitory effect can be seen on both M1 proliferation and M2 proliferation when using a co-culture with PBMCs and MSCs.

Assay controls (CFSE proliferation): CsA and Prednisolone treated cells show no effect on % T helper cells or cytotoxic T cells after 72 hours of incubation. Prednisolone treated cells show no effect on the M1 or M2 expression levels although, CsA treated cells show a shift towards M2. No effect can be seen on T cell proliferation when using a co-culture with PBMCs and MSCs after CsA treatment. Prednisolone inhibits proliferation of cytotoxic T cells. No effect can be seen on M1 or M2 proliferation after CsA or Prednisolone treatment.

Results from cytokine analysis after co-culturing PBMCs with MSCs or reference drugs are shown in FIG. 40. Results from cytokine analysis in MSC culture supernatant (CM) or in PBMCs and MSCs not grown in co-culture are shown in FIG. 41. VEGF is produced by MSCs but not by PBMCs (FIG. 40I, 41L). IGF and b-NGF are produced by MSCs but not by PBMCs or AD-MSCs (FIG. 40A. 41D, 40L, 41K). IL-10, TNF-α, and IL12/IL23p40 are produced by stimulated PBMCs and downregulated in co-culture of PBMCs and MSCs at all ratios (FIG. 40C, 40J, 40K). CXCL9, HGF and IL-18 are produced by stimulated PBMCs and downregulated in co-culture of PBMCs and MSCs in a dose-dependent manner (FIG. 40B, 40E, 40G). Since PBMC:MSC ratios used in this experiment were: 1:2.5, 1:5, and 1:10, more MSCs than PBMCs were added per well and results from CXCL9, HGF and IL-18 are therefore difficult to interpret. No inhibitory effect can be seen on IFN-g (FIG. 40H).

No conclusions can be drawn from the figure with IFN-α (FIG. 40D). Outliers have to be removed and figure redone before analysis can be made. No conclusions can be drawn from the figure with IL-6 (FIG. 40F). It appears as if the assay sensitivity could be wrongly calibrated. It appears as if maximal values are reached for all conditions. In FIG. 41I it is shown that the pro-inflammatory cytokine IL-6 is produced by PBMCs and AD-MSCs, but not by test item MSCs.

CsA also inhibits CXCL9 (FIG. 40B), IL-10 (FIG. 40C), HGF (FIG. 40E), IL-6 (FIG. 40F), IL-18 (FIG. 40G), TNF-α (FIG. 40J) and IL-13/IL-23 p40 (FIG. 40K) levels in supernatant. No inhibitory effect can be seen on the other analysed cytokines. Prednisolone also inhibits HGF (FIG. 40E), IL-6 (FIG. 40F), TNF-α (FIG. 40J) and IL-12/IL-23 p40 (FIG. 40K) levels in supernatant. No inhibitory effect can be seen on the other analysed cytokines.

### Items

Item 1. Isolated term amniotic fluid (TAF) mesenchymal stem cells (MSCs) for use in modulating an immune response in a subject after tissue and/or organ transplantation.

Item 2. An anti-rejection composition comprising isolated TAF MSCs for use in modulating an immune response in a subject after tissue and/or organ transplantation.

Item 3. The isolated TAF MSCs and/or the anti-rejection composition according to any one of Items 1 or 2, wherein the immune response is inflammation.

Item 4. The isolated TAF MSCs and/or the anti-rejection composition according to any one of the preceding Items, wherein the inflammation is tissue/organ-specific inflammation.

Item 5. The isolated TAF MSCs and/or the anti-rejection composition according to any one of the preceding Items, wherein the tissue/organ-specific inflammation is one or more of the group consisting of lung, kidney, neural, skin, liver, heart (and heart valves), trachea, body parts (such as limbs/digits), pancreas, intestine colon systemic inflammation and any combination thereof.

Item 6. Isolated TAF MSCs or an anti-rejection composition comprising isolated TAF MSCs for use in treating/reducing/preventing transplant rejection of a donor tissue and/or a donor organ.

Item 7. The isolated TAF MSCs or the anti-rejection composition according to Item 6, wherein:
a. the donor tissue and/or donor organ was obtained from a donor treated with isolated TAF MSCs;
b. the donor tissue and/or the donor organ has been pre-treated ex-vivo with isolated TAF MSCs;
c. the donor tissue and/or the donor organ was transported ex-vivo in a conditioning media, wherein said conditioning media comprises isolated TAF MSCs and/or a composition comprising isolated TAF MSCs, and/or
d. wherein the isolated TAF MSCs (or a portion thereof, if mixed population) comprise tissue/organ-specific markers, preferably wherein the tissue/organ-specific markers correspond to said donor tissue/donor organ.

Item 8. The isolated TAF MSCs or composition comprising isolated TAF MSCs according to any one of the preceding Items, wherein the donor tissue/donor organ is from a non-living subject, preferably wherein the non-living subject is the same species as the intended recipient of the donor tissue/donor organ (for example the organ may be obtained from a non-living human (corpse/cadaver) for transplantation in a human in need thereof).

Item 9. The isolated TAF MSCs or composition comprising isolated TAF MSCs according to any one of the preceding Items, wherein the donor tissue/donor organ is selected from the group consisting of a lung, kidney, neural, skin, liver, heart (and heart valves), trachea, body parts (such as limbs/digits), pancreas, intestine and colon.

Item 10. The isolated TAF MSCs or composition comprising isolated TAF MSCs according to any one of the preceding Items, wherein the donor tissue/donor organ remains viable for at least at least 1 hour, such as 2 hours, e.g. 4 hours, such as 6 hours, e.g. 8 hours, such as 10 hours, e.g. 12 hours, such as 14 hours, e.g. 16 hours, such as 18 hours, e.g. 20 hours, such as 22 hours, e.g. 1 days, such as 2 days post-transplantation.

Item 11. The isolated TAF MSCs or composition comprising isolated TAF MSCs according to any one of the preceding Items, wherein isolated TAF MSCs and/or composition have been introduced to an ex-vivo donor tissue and/or ex-vivo donor organ before and/or are introduced during transplantation to the recipient.

Item 12. The isolated TAF MSCs or composition comprising isolated TAF MSCs according to any one of the preceding Items, wherein said isolated TAF MSCs or composition comprising isolated TAF MSCs is administered in combination with a further agent.

Item 13. The isolated TAF MSCs or composition comprising isolated TAF MSCs according to any one of the preceding Items, wherein the further agent is administered as part of the composition comprising isolated TAF MSCs.

Item 14. The isolated TAF MSCs or composition comprising isolated TAF MSCs according to any one of Items 12 or 13, wherein the further agent is selected from the group consisting of anti-inflammatory agents, immunosuppressive agents, anti-rejection agents/drugs (e.g. prednisone, tacrolimus, etc) and any combinations thereof.

Item 15. The isolated TAF MSCs or composition comprising isolated TAF MSCs according to any one of the preceding Items, wherein the isolated TAF MSCs or composition comprising isolated TAF MSCs is administered:
a. more than once;
b. at a concentration of 1-2 million cells per kg of the recipient; and/or
c. intravenously.

Item 16. The isolated TAF MSCs or composition isolated TAF MSCs or composition comprising isolated TAF MSCs according to any preceding Items, wherein the isolated TAF MSCs are derived from an MHC/HLA-matched donor.

Item 17. The isolated TAF MSCs or composition comprising isolated TAF MSCs according to any one of the preceding Items, wherein the isolated TAF MSCs are:
a. a clonal population;
b. a mix of clonal populations;
c. heterogeneous/homogeneous;
d. in a single-cell suspension/pelleted;
e. are capable of forming colony forming units (CFU) in culture;
f. functionally characterised (e.g. release of cytokines implicated in growth stimulation or suppression; differentiation into osteogenic, chondrogenic or adipogenic cell lineages);
g. have been pre-sorted or enriched to contain markers of interest;
h. passaged 1, 2, 3, 4, 5, 6, etc times; and/or
i. in a frozen state (and require thawing before use).

Item 18. The isolated TAF MSCs or composition comprising isolated TAF MSCs according to any one of the preceding Items, wherein the isolated TAF MSCs comprise (or have been enriched/selected to comprise):
(i) at least one surface marker selected from the group consisting of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
(ii) at least one surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
(iii) at least one surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1;
(iv) at least one surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2; or
(v) at least one surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.

Item 19. The isolated TAF MSCs or composition comprising isolated TAF MSCs according to any one of the preceding Items, wherein the isolated TAF MSCs are between 15-25 μm diameter, preferably between 18-22 μm diameter.

Item 20. The isolated TAF MSCs or composition comprising isolated TAF MSCs according to any one of the preceding Items, wherein the isolated TAF MSCs comprise lower actin expression or fewer vesicles at the surface compared with adult MSCs.

Item 21. The isolated TAF MSCs or composition for use according to any one of the preceding Items, wherein the TAF-MSCs are lung TAF-MSCs, kidney TAF-MSCs, neural TAF-MSCs, skin TAF-MSCS, or any combination thereof.

Item 23. The isolated TAF MSCs or composition for use according to any one of the preceding Items, wherein the TAF-MSCs are at least 10%, 20%, 30%, 40%, 50%, 60%, 70%, 80%, 90%, 95%, or more, lung TAF-MSCs.

Item 23. Isolated term amniotic fluid (TAF) mesenchymal stem cells (MSCs) for use in preventing and/or treating graft dysfunction and/or graft versus host disease (GVHD) in a subject after tissue and/or organ transplantation.

Item 24. An anti-rejection composition comprising isolated TAF MSCs for use in preventing and/or treating graft dysfunction and/or graft versus host disease (GVHD) in a subject after tissue and/or organ transplantation.

### REFERENCES

The listing or discussion of an apparently prior-published document in this specification should not necessarily be taken as an acknowledgement that the document is part of the state of the art or is common general knowledge. The references disclosed, to the extent that they provide exemplary procedural or other details supplementary to those set forth herein, are specifically incorporated herein by reference.
Force, A D T., Ranieri, V M., Rubenfeld, G D., Thompson, B T., Ferguson, N D., Caldwell, E., 2012, Acute respiratory distress syndrome: the Berlin Definition, JAMA, 307(23):2526-33.
Mariscal, A., Cypel, M., Keshavjee, S., 2017, Ex Vivo Lung Perfusion, Curr Transpl Rep, 4:149-158.
Mariscal, A., Caldarone, L., Tikkanen, J., Nakajima, D., Chen, M., Yeung, J., 2018, Pig lung transplant survival model, Nat Protoc., 13(8): 1814-28.
Mo, M., Zhou, Y., Li, S., Wu, Y., 2017, Three-Dimensional Culture Reduces Cell Size By Increasing Vesicle Excretion, Stem Cells, 2018(36): 286-292.
Mordant, P., Nakajima, D., Kalaf, R., Iskender, I., Maahs, L., Behrens, P., Coutinho, R., Iyer, R. K., Davies, J. E., Cypel, M., Liu, M., Waddell, T. K., Keshavjee, S., 2016, Mesenchymal stem cell treatment is associated with decreased perfusate concentration of interleukin-8 during ex vivo perfusion of donor lungs after 18-hour preservation, J Heart Lung Transplant, 35(10): 1245-1254.
Matute-Bello G, Downey G, Moore B B, Groshong S D, Matthay M A, Slutsky A S, 2011, An official American Thoracic Society workshop report: features and measurements of experimental acute lung injury in animals, Am J Respir Cell Mol Biol, 44(5): 725-38.
Nakajima, D., Watanabe, Y., Ohsumi, A., Pipkin, M., Chen, M., Mordant, P., Kanou, T., Saito, T., Lam, R., Coutinho, R., Caldarone, L., Juvet, S., Martinu, T., Iyer, R., Davies, J., Hwang, D., Waddell, T., Cypel, M., Liu, M., Keshavjee, S., 2019, Mesenchymal stromal cell therapy during ex vivo lung perfusion ameliorates ischemia-reperfusion injury in lung transplantation, The Journal of Heart and Lung Transplantation, 38(11): 1214-1223.
Okamoto, T., Niikawa, H., Ayyat, K., Sakanoue, I., Said, S., McCurry, K., 2019, Machine Perfusion of Lungs, Current Transplantation Report, 6:251-264.
Pacienza, N., Santa-Cruz, D., Malvicini, R., Robledo, O., Lemus-Larralde, G., Bertolotti, A., Marcos, M., and Yannarelli, G., 2019, Mesenchymal Stem Cell Therapy Facilitates Donor Lung Preservation by Reducing Oxidative Damage during Ischemia, Stem Cells Int, Volume 2019, Article ID 8089215, 13 pages.
Pan, X., Yang, J., Fu, S., Zhao, H., 2018, Application of ex vivo lung perfusion (EVLP) in lung transplantation, Journal of Thoracic Disease, 10(7): 4637-4642.
Snell G I, Yusen R D, Weill D, Strueber M, Garrity E, Reed A, 2017, Report of the ISHLT Working Group on Primary Lung Graft Dysfunction, part I: Definition and grading-A 2016 Consensus Group statement of the International Society for Heart and Lung Transplantation, J Heart Lung Transplant, 36(10):1097-103.
Stenlo M, Hyllen S, Silva IAN, Bolukbas D A, Pierre L T, Hallgren O, 2020, Increased particle flow rate from airways precedes clinical signs of ARDS in a porcine model of LPS-induced acute lung injury, Am J Physiol Lung Cell Mol Physiol., 318(3):L510-L517.
Tian, W., Liu, Y., Zhang, B., Dai, X., Li, G., Li, X., Zhang, Z., Du, C., Wang, H., 2014, Infusion of Mesenchymal Stem Cells Protects Lung Transplants from Cold Ischemia-Reperfusion Injury in Mice, Lung, DOI 10.1007/s00408-014-9654-x.
Van Raemdonck D, Neyrinck A, Cypel M, Keshavjee S., 2015, Ex-vivo lung perfusion, Transpl Int, 28(6):643-56.
Yeung J C, Cypel M, Massad E, Keshavjee S., 2011, Ex vivo lung perfusion and reconditioning. Multimed Man Cardiothorac Surg., 2011(418): mmcts 2009 004242.

1. A method of modulating an immune response in a subject after tissue and/or organ transplantation comprising administering to said subject isolated term amniotic fluid (TAF) mesenchymal stem cells (MSCs) or a composition comprising isolated TAF MSCs.

2-24. (canceled)

25. The method of claim 1, wherein the subject is administered a composition comprising isolated TAF MSCs.

26. The method of claim 1, wherein the immune response is inflammation.

27. The method of claim 26, wherein the inflammation is tissue or organ-specific inflammation.

28. The method of claim 1, wherein the tissue or organ-specific inflammation is inflammation of the lung, kidney, neural inflammation, skin, liver, heart, heart valve, trachea, a body part, pancreas, intestine, colon or systemic inflammation or any combination thereof.

29. A method of treating or inhibiting transplant rejection of a donor tissue and/or donor organ, graft dysfunction and/or graft versus host disease (GVHD) comprising administering to said subject isolated TAF MSCs or a composition comprising isolated TAF MSCs.

30. The method of claim 29, wherein:
a. the donor tissue and/or donor organ was obtained from a donor that received isolated TAF MSCs;
b. the donor tissue and/or the donor organ has been contacted ex-vivo with isolated TAF MSCs;
c. the donor tissue and/or the donor organ was transported ex-vivo in a conditioning media, wherein said conditioning media comprises isolated TAF MSCs and/or a composition comprising isolated TAF MSCs, and/or
d. wherein the isolated TAF MSCs or a portion thereof comprise tissue or organ-specific markers.

31. The method of claim 1, wherein the donor tissue or donor organ is from a non-living subject.

32. The method of claim 1, wherein the donor tissue or donor organ is selected from the group consisting of a lung, kidney, neural tissue, skin, liver, heart, heart valve, trachea, a body part, pancreas, intestine and colon.

33. The method of claim 1, wherein the donor tissue or donor organ remains viable for at least at least 1 hour post-transplantation.

34. The method of claim 1, wherein the isolated TAF MSCs and/or the composition have been introduced to an ex-vivo donor tissue and/or ex-vivo donor organ before transplantation and/or are introduced during transplantation to the recipient.

35. The method of claim 1, wherein said isolated TAF MSCs or composition comprising isolated TAF MSCs is administered in combination with an additional agent.

36. The method of claim 35, wherein the additional agent is administered as part of the composition comprising isolated TAF MSCs.

37. The method of claim 35, wherein the additional agent is selected from the group consisting of anti-inflammatory agents, immunosuppressive agents, anti-rejection agents, anti-rejection drugs, and any combinations thereof.

38. The method of claim 1, wherein the isolated TAF MSCs or composition comprising isolated TAF MSCs is administered:
more than once;
at a concentration of 1-2 million cells per kg of the recipient; and/or
intravenously.

39. The method of claim 1, wherein the isolated TAF MSCs are derived from an MHC/HLA-matched donor.

40. The method of claim 1, wherein the isolated TAF MSCs are:
a clonal population;
a mix of clonal populations;
heterogeneous or homogeneous;
in a single-cell suspension or pelleted;
are capable of forming colony forming units (CFU) in culture;
functionally characterised;
have been pre-sorted or enriched to contain markers of interest;
passaged; and/or
in a frozen state.

41. The method of claim 1, wherein the isolated TAF MSCs comprise:
(i) at least one surface marker selected from the group consisting of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
(ii) at least one surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
(iii) at least one surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, LICAM, GFRA1, IGSF3, TNF, MMP7, FOLR1, TGFA, C3, TNFSF10, PDGFB and WWC1;
(iv) at least one surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2; or
(v) at least one surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.

42. The method of claim 1, wherein the isolated TAF MSCs are between 15-25 μm diameter.

43. The method of claim 1, wherein the isolated TAF MSCs comprise lower actin expression or fewer vesicles at the surface compared with adult MSCs.

44. The method of claim 1, wherein the TAF-MSCs are lung TAF-MSCs, kidney TAF-MSCs, neural TAF-MSCs, skin TAF-MSCS, or any combination thereof.

45. The method of claim 1, wherein the TAF-MSCs are at least 10%, 20%, 30%, 40%, 50%, 60%, 70%, 80%, 90%, 95%, or more, lung TAF-MSCs.
